(19) United States
(12) Patent Application Publication
(10) Pub. No.: US 2010/0278775 A1
(43) Pub. Date: Nov. 4, 2010

(54) Plant Bioreactor For The Production Of Interleukin-24 Cytokine

(75) Inventors: Rima Menassa, London (CA); Alexandra Reid, London (CA)

Correspondence Address: SCHWEGMAN, LUNDBERG & WOESSNER, P.A., P.O. BOX 2938, MINNEAPOLIS, MN, 55402, US

(73) Assignee: Her Majesty the Queen in Right of Canada, as Re- presented by the Minister of Agriculture and Agri-, London (CA)

(21) Appl. No.: 12/733,075

(22) PCT Filed: Aug. 8, 2008
(86) PCT No.: PCT/CA2008/001447

§ 371 Date: Jun. 29, 2010

Related U.S. Application Data
(60) Provisional application No. 60/963,935, filed on Aug. 8, 2007.

(51) Int. Cl. A61K 38/20; C12N 15/24; A01H 5/00; C12N 5/04; C12P 21/00; C12N 15/82; A61P 35/00

(52) U.S. Cl. 424/85.2; 536/23.5; 800/298; 435/419; 435/69.52; 800/288

(57) ABSTRACT

The production of interleukin-24 (IL-24) cytokine in plants is described. A plant optimized nucleic acid molecule encoding a IL-24 polypeptide is disclosed. Also described are genetic constructs comprising a regulatory region operably linked to a plant optimized nucleic acid molecule encoding a IL-24 polypeptide. The regulatory region may be an inducible promoter. Also disclosed are methods of transforming a plant, portion thereof, or plant cell with the genetic construct and method of using a plant, a portion of a plant or a plant cell that express IL-24.

### FIELD OF INVENTION

The present invention relates to the use of plants for the production of the interleukin-24 (IL-24) cytokine. More specifically this invention relates to the optimization and control of expression of recombinant interleukin-24 in plants and plant cell suspensions.

### BACKGROUND OF THE INVENTION

Studies conducted during the past 10 years clearly demonstrate that plants can be engineered to produce a wide range of pharmaceutical proteins in a broad array of crop species and tissues (Kusnadi et al. 1997). Evidence of the utility of tobacco plants as bioreactors for proteins of medical relevance has been demonstrated by the expression and assembly of a multi-chain secretory antibody that are effective in human clinical trials (Ma et al. 1995, 1998). Further proof came with the expression of murine GAD65 and its application to diabetes prevention in NOD mouse (Ma et al. 1997, 2004).

Plants offer several distinct advantages over conventional expression systems for the production of recombinant proteins. Since plants are higher eukaryotes they can properly fold and assemble proteins in a manner similar to mammalian cells. In addition, plant systems are free of mammalian pathogens. Unlike fermentation-based bacterial and mammalian cell systems, protein production in plants is not restricted by physical facilities. Agricultural scale production ensures the availability of recombinant proteins in amounts sufficient for extensive clinical studies and therapeutic use. Although extraction and purification costs may be similar to other systems, the cost of producing the raw plant material is estimated to be significantly lower than that of fermentation (Kusnadi et al. 1997).

As the production of plant recombinant proteins moves from the bench to pilot scale, practical issues, such as safety and containment, must be considered. A non-food crop platform with limited capacity for the spread of pollen or seed addresses environmental and safety concerns. Tobacco is a non-food crop that has been the subject of many years of breeding and agronomic research that can be used as a strong base for a field production system for molecular farming. Menassa et al. (2001) describe a production system that is based on hybrids between male-sterile, low-alkaloid (MSLA) females and homozygous transgenic lines. The low-alkaloid background genotype is optimized for agricultural production, introduces the concept that genetic backgrounds can be modified to address downstream needs and may also be more amenable to direct oral administration than normal cultivars. Seed can be produced in containment with homozygous transgenics as males, so that the resultant hybrids express the transgene in every plant. The system is based on leaves, not seeds or tubers, which limits the potential for escape of the transgene. Male sterility further reinforces that containment. The plants are grown at a high density to maximize biomass yield and are harvested after 30-40 days, which is prior to flower appearance allowing the system to be adapted to a broad range of production environments. Multiple staggered plantings ensure the availability of plant material for processing throughout the growing season.

Human melanoma differentiation associated gene-7 (mda-7), also known as interleukin-24 (IL-24), is a novel gene with tumour suppressor properties in a variety of cancers including carcinomas of the prostate (Saito et al, 2005), ovary (Gopalan et al, 2003), lung (Saeki et al., 2002), breast (Su et al., 1998), kidney (Yacoub et al., 2003), colon (Huang et al., 2001), and melanoma (Lebedeva et al., 2002). The IL-24 gene encodes a protein of 206 amino acids with a predicted size of 23.8 kDa. The IL-24 gene has a tissue-specific expression pattern, restricted to melanocytes and certain lymphocytes and leukocyte subtypes (Huang et al., 2001; Garn et al., 2002).

Over-expression of IL-24 induces apoptosis in cancer cells but not in normal cells. IL-24 is able to induce transformed cell-type apoptosis in almost every type of cell line so far tested (Lebedeva et al., 2005). Upon exposure to high levels of IL-24, a wide array of proapoptotic signals and pathways are activated in oncogenic cells, while normal counterparts remain unaffected (Fisher et al., 2003; Lebedeva et al., 2003; Sauane et al., 2003; Chada et al., 2004). These results were extended with successful completion of a phase I clinical trial, and with positive results in an ongoing phase VII clinical trial, indicating safety and potential anti-tumour efficacy of this gene when delivered via a non-replicating adenoviral vector (Nemunaitis, 2003; Cunningham et al., 2005; Tong et al., 2005). These clinical trials have demonstrated that adenoviral delivery of IL-24 is well tolerated when administered via intratumoral injection. However the levels of IL-24 delivered are not sufficient, and alternate delivery vehicles and higher levels of IL-24 protein may result in greater clinical benefits.

Colorectal cancer is the leading cause of cancer-related mortality in western countries. Recurrent or metastatic disease occurs in half of the new cases within one year of diagnosis and median survival does not exceed 18 months. Colorectal cancer represents an optimal model for primary and secondary prevention, given the availability of effective screening procedures and of a well-defined multi-step carcinogenic pathway (Serrano et al., 2004). In colorectal cancer, patients displaying a high degree of microsatellite instability (MSI-H) have an improved prognosis compared to microsatellite stable (MSS) patients. Banerjea et al. (2004) have shown increased mRNA levels of IL-24 and other pro-inflammatory cytokines in MSI-H as compared to MSS, probably as an immune response to MSI-H. Increased levels of pro-inflammatory cytokines indicate an activated anti-tumour immune response.

There is a need in the art for a method of making IL-24 in large quantities and at low cost. Further there is a need in the art for making biologically active IL-24 in large quantities and low cost, that do not require purification or further processing prior to being administered to a subject.

### SUMMARY OF THE INVENTION

The present invention relates to the use of plants for the production of interleukin-24 (IL-24) cytokine.

The present invention provides a plant optimized nucleic acid molecule encoding a IL-24 polypeptide. The plant optimized nucleic acid molecule may comprise a nucleotide sequence as set forth in SEQ ID NO: 3, a nucleotide sequence that exhibits from about 70% to about 100% sequence identity with the nucleotide sequence of SEQ ID NO:3, or a nucleotide sequence that hybridizes to the nucleotide sequence of SEQ ID NO: 3 under conditions of high stringency. Furthermore, the IL-24 polypeptide may comprise an amino acid sequence as set forth in SEQ ID NO: 4.

The present invention also provides a genetic construct (construct A) comprising a regulatory region operably linked to a plant optimized nucleic acid molecule encoding a IL-24 polypeptide. The plant optimized nucleic acid molecule encoding a IL-24 polypeptide may comprise a nucleotide sequence as set forth in SEQ ID NO: 3, a nucleotide sequence that exhibits from about 70% to about 100% sequence identity with the nucleotide sequence of SEQ ID NO:3, or a nucleotide sequence that hybridizes to the nucleotide sequence of SEQ ID NO: 3 under conditions of high stringency. Furthermore, the IL-24 polypeptide may comprise an amino acid sequence as set forth in SEQ ID NO: 4.

The present invention pertains to a genetic construct (construct A) described above wherein the regulatory region is an inducible promoter, such as but not limited a Heat Shock Protein (HSP). The genetic construct (construct A) described above may further comprising a nucleotide sequence encoding an elastin-like polypeptide (ELP) or a fragment thereof, a targeting motif, or both. The targeting motif may be an endoplasmic reticulum (ER) targeting motif, such as but not limited to KDEL. Alternatively, the targeting motif may be a peroxisome targeting motif, such as but not limited to SKL.

The present invention provides a genetic construct (construct A) as herein described wherein the native 5′-untranslated region (5′UTR) of the plant optimized nucleic acid molecule encoding a IL-24 polypeptide is replaced with a translation enhancer, such as but not limited to a tCUP promoter. Alternatively or additionally, the native signal peptide sequence of the plant optimized nucleic acid molecule encoding a IL-24 polypeptide may be replaced with a heterologous signal/transit peptide. The signal/transit peptide may be a chloroplast targeting peptide, such as but not limited to a ribulose-1,5-bisphosphate carboxylase (Rubisco) transit peptide.

The present invention also provides a genetic construct (construct B) comprising an inducible promoter operably linked to a plant optimized nucleic acid molecule encoding a IL-24 polypeptide. The plant optimized nucleic acid molecule encoding a IL-24 polypeptide or fragment or variant thereof may comprise a nucleotide sequence as set forth in SEQ ID NO: 3, a nucleotide sequence that exhibits from about 70% to about 100% sequence identity with the nucleotide sequence of SEQ ID NO:3, or a nucleotide sequence that hybridizes to the nucleotide sequence of SEQ ID NO: 3 under conditions of high stringency. Furthermore, the IL-24 polypeptide may comprise an amino acid sequence as set forth in SEQ 1D NO: 4. The inducible promoter may be a Heat Shock Protein (HSP).

The present invention pertains to a genetic construct (construct B) described above wherein the regulatory region is an inducible promoter, such as but not limited a Heat Shock Protein (HSP). The genetic construct (construct A) described above may further comprising a nucleotide sequence encoding an elastin-like polypeptide (ELP) or a fragment thereof, a targeting motif, or both. The targeting motif may be an endoplasmic reticulum (ER) targeting motif, such as but not limited to KDEL. Alternatively, the targeting motif may be a peroxisome targeting motif, such as but not limited to SKL.

The present invention also provides a genetic construct (construct C) comprising:
i) an inducible promoter operably linked to a plant optimized nucleic acid molecule encoding a IL-24 polypeptide or fragment or variant thereof;
ii) a nucleotide sequence encoding an elastin-like polypeptide (ELP) or fragment or variant thereof; and
iii) an endoplasmic reticulum (ER) targeting motif.

The plant optimized nucleic acid molecule encoding a IL-24 polypeptide may comprise a nucleotide sequence as set forth in SEQ ID NO: 3, a nucleotide sequence that exhibits from about 70% to about 100% sequence identity with the nucleotide sequence of SEQ ID NO:3, or a nucleotide sequence that hybridizes to the nucleotide sequence of SEQ ID NO: 3 under conditions of high stringency. Furthermore, the IL-24 polypeptide may comprise an amino acid sequence as set forth in SEQ ID NO: 4. The genetic construct as hereinbefore describe (construct C) may comprise SEQ ID NO: 138.

The present invention also provides a genetic construct (construct D) comprising a nucleotide sequence selected from the group consisting of:
a. SEQ ID NO: 5, SEQ ID NO: 64, SEQ ID NO: 67, SEQ ID NO: 111, SEQ ID NO: 114, SEQ ID NO: 118, SEQ ID NO: 120, SEQ ID NO: 122, SEQ ID NO: 131, SEQ ID NO: 136, or SEQ ID NO: 138;
b. a nucleotide sequence that exhibits from about 70% to about 100% sequence identity with the nucleotide sequence of SEQ ID NO: 5, SEQ ID NO: 64, SEQ ID NO: 67, SEQ ID NO: 111, SEQ ID NO: 114, SEQ ID NO: 118, SEQ ID NO: 120, SEQ ID NO: 122, SEQ ID NO: 131, SEQ ID NO: 136, or SEQ ID NO: 138; and
c. a nucleotide sequence that hybridizes to the nucleotide sequence of SEQ ID NO: 5, SEQ ID NO: 64, SEQ ID NO: 67, SEQ ID NO: 111, SEQ ID NO: 114, SEQ ID NO: 118, SEQ ID NO: 120, SEQ ID NO: 122, SEQ ID NO: 131, SEQ ID NO: 136 or SEQ ID NO: 138 under conditions of high stringency.

The present invention further provides a genetic construct comprising a nucleotide sequence as set forth in SEQ ID NO: 136 or SEQ ID NO: 138, a nucleotide sequence that exhibits from about 70% to about 100% sequence identity with the nucleotide sequence of SEQ ID NO: 136 or SEQ ID NO: 138, or a nucleotide sequence that hybridizes to the nucleotide sequence of SEQ ID NO: 136 or SEQ ID NO: 138 under conditions of high stringency.

The genetic construct of the present invention may express a fusion protein comprising IL-24 or a fragment thereof and a fusion partner. The fusion partner may be a reporter protein or a fragment thereof, an elastin-like polypeptide (ELP) or a fragment thereof, or both a reporter protein and an ELP or fragments thereof. The reporter protein may be a green fluorescent protein. The fusion protein may be a polypeptide selected from the group consisting of SEQ ID NO: 6, SEQ ID NO: 65, SEQ ID NO: 68, SEQ ID NO: 112, SEQ ID NO: 115, SEQ ID NO: 119, SEQ ID NO: 121, SEQ ID NO: 123, SEQ ID NO: 132, SEQ ID NO: 137, and SEQ ID NO: 139.

The present invention also provides a vector comprising the genetic construct as hereinbefore described. The vector may be selected from the group consisting of pC24-3, pC24-6, pC24-1, pC24-5, pC24-4, pC24-15, pC24-19, pC24-20, pC24-14, pRD24-22, and pRD24-23.

The present invention provides a plant, a portion thereof, or a plant cell comprising a regulatory region operably linked to a plant optimized nucleic acid molecule encoding an IL-24 polypeptide. The plant optimized nucleic acid molecule encoding the IL-24 polypeptide may comprise a nucleotide sequence as set forth in SEQ ID NO: 3, a nucleotide sequence that exhibits from about 70% to about 100% sequence identity with the nucleotide sequence of SEQ ID NO:3, or a nucleotide sequence that hybridizes to the nucleotide sequence of SEQ ID NO: 3 under conditions of high stringency. The regulatory region may comprise an inducible promoter. Furthermore, the IL-24 polypeptide may comprise an amino acid sequence as set forth in SEQ ID NO: 4.

The present invention also provides a plant, or portion thereof, comprising construct A, construct B, construct C or construct D, as hereinbefore described. The present invention also provides a plant cell comprising construct A, construct B, construct C or construct D as hereinbefore described.

The present invention also pertains to a plant cell culture, comprising a regulatory region operably linked to a plant optimized nucleic acid molecule encoding a IL-24 polypeptide. The plant optimized nucleic acid molecule encoding an IL-24 polypeptide or fragment or variant thereof may comprise a nucleotide sequence as set forth in SEQ ID NO: 3, a nucleotide sequence that exhibits from about 70% to about 100% sequence identity with the nucleotide sequence of SEQ ID NO:3, or a nucleotide sequence that hybridizes to the nucleotide sequence of SEQ ID NO:3 under conditions of high stringency. The regulatory region may comprise an inducible promoter. Furthermore, the IL-24 polypeptide may comprise an amino acid sequence as set forth in SEQ ID NO: 4.

The present invention also provides a plant cell culture comprising construct A, construct B, construct C or construct D as hereinbefore described.

The present invention also contemplates a plant, plant cells, plant cell culture, tissues, seeds comprising IL-24 cytokine.

The plant cell, plant cell culture, plant, or portion thereof, may be selected from the group consisting of canola, Brassica spp., maize, tobacco spp., alfalfa, potato, ginseng, pea, oat, rice, soybean, wheat, barley, sunflower, and cotton. In an embodiment which is not meant to be limiting, the plant cell, plant cell culture, plant, or portion thereof is a tobacco. The tobacco plant may be a low-nicotine, low-alkaloid tobacco, such as but not limited to 81V-9.

Also provided in the present invention is a method (A) of producing an IL-24 cytokine for oral administration comprising, either transforming a plant or portion thereof with a regulatory region operably linked to a plant optimized nucleic acid molecule encoding an IL-24 polypeptide, or providing a plant or a portion thereof that comprises a regulatory region operably linked to a plant optimized nucleic acid molecule encoding an IL-24 polypeptide, growing the plant of the portion thereof, and expressing the IL-24 cytokine in the plant, or a portion thereof. The plant optimized nucleic acid molecule encoding a IL-24 polypeptide may comprise a nucleotide sequence as set forth in SEQ ID NO: 3, a nucleotide sequence that exhibits from about 70% to about 100% sequence identity with the nucleotide sequence of SEQ ID NO:3, or a nucleotide sequence that hybridizes to the nucleotide sequence of SEQ ID NO:3 under conditions of high stringency. The regulatory region may comprise an inducible promoter. Furthermore, the IL-24 polypeptide may comprise an amino acid sequence as set forth in SEQ ID NO: 4.

Also according to the present invention, there is provided a method (B) for the production of IL-24, the method comprising,
i) selecting a low nicotine, low alkaloid tobacco plant;
ii) transforming the plant with a vector containing a regulatory region operably linked to a plant optimized nucleic acid molecule encoding an IL-24 polypeptide allowing expression of the IL-24 polypeptide, thereby producing a transformed plant;
iii) growing and harvesting the transformed plant containing the IL-24 polypeptide, and;
iv) obtaining plant tissues from the transformed plant that comprise the IL-24.

Also according to the present invention, there is provided a method (C) for the production of IL-24, the method comprising,
i) providing a low nicotine, low alkaloid tobacco plant comprising a nucleic acid containing a regulatory region operably linked to a plant optimized nucleic acid molecule encoding an IL-24 polypeptide allowing expression of the IL-24 polypeptide; and
ii) growing and harvesting the low nicotine, low alkaloid tobacco plant containing the IL-24 polypeptide, and;
iv) obtaining plant tissues from the low nicotine, low alkaloid tobacco plant that comprise the IL-24.

Preferably the leaves of the low-nicotine, low-alkaloid tobacco plant comprise, a nicotine concentration (wt/wt) which is less than about 10%, more preferably less than about 5%, still more preferably less than about 2.6% the nicotine concentration (wt/wt) of Delgold tobacco plants grown under the same conditions, and an alkaloid concentration (wt/wt) which is less than about 10%, more preferably less than about 5%, still more preferably less than about 0.28% the alkaloid concentration (wt/wt) of Delgold tobacco plants grown under the same conditions. The alkaloid may consist of, but is not limited to, nicotine, myosmine, anabasine and anatabine.

The present invention also provides a method of producing an IL-24 cytokine for oral administration comprising, growing a plant, or portion thereof with the genetic construct as described above, expressing the IL-24 cytokine in the plant, or a portion thereof, and harvesting the plant, or a portion thereof comprising the IL-24 cytokine, and using the plant, a portion of the plant, an crude extract, or a purified extract comprising the IL-24 for oral administration.

Also according to the present invention, there is provided a composition comprising plant expressed IL-24.

The present invention pertains to a method of treating cancer in a subject comprising, orally administering to the subject a composition comprising a plant-expressed IL-24. The cancer may be colorectal cancer.

Further, the present invention provides a method of treating cancer comprising orally administering a plant or portion thereof comprising IL-24 to the subject. The cancer may be colorectal cancer.

The present invention pertains to a use a composition comprising a plant-expressed IL-24 for treating cancer in a subject, wherein the composition is orally administrable. The cancer may be colorectal cancer.

Further the present invention pertains to use of a plant or portion thereof comprising a plant-expressed IL-24 for treating cancer in a subject, wherein the plant or portion thereof is orally administrable. The cancer may be colorectal cancer.

The invention is in part based on the finding that adenoviral delivery of IL-24 to melanoma tumours induced cell death of cancer cells. Because non replicating adenovirus vectors are used for this treatment method, an increased concentration of IL-24 may produce a better therapeutic effect. Use of plants as a bioreactor for the production of IL-24 polypeptide may produce large quantities of IL-24. Either the purified IL-24 protein or plant material containing the IL-24 protein can be orally administered to a subject. In this way, dosage can be better controlled. Furthermore, oral delivery of IL-24 for the treatment of colorectal cancer is appealing as it eliminates or alleviates the cytotoxic effects associated with other routes of IL-24 administration.

This summary of the invention does not necessarily describe all necessary features of the invention, but that the invention may also reside in a sub-combination of the described features.

### BRIEF DESCRIPTION OF THE DRAWINGS

These and other features of the invention will become more apparent from the following description in which reference is made to the appended drawings wherein:

FIG. 1A shows a nucleotide sequence (SEQ ID NO: 1) and deduced amino acid sequence (SEQ ID NO: 2) of Homo sapiens interleukin 24 (IL-24) (Accession number NM_{—}006850). Small letters without amino acid residues represent the upstream 5′ untranslated region (5′UTR) and bold residues represent the signal peptide. FIG. 1B shows a nucleotide sequence (SEQ ID NO: 3) and deduced amino acid sequence (SEQ ID NO: 4) for a tobacco codon-optimized synthetic gene of IL-24. Small letters without amino acid residues represent the upstream 5′ untranslated region and bold residues represent the signal peptide. FIG. 1C shows an alignment of Homo sapiens IL-24 nucleotide sequence (SEQ ID NO: 1), and a plant optimized IL-24 nucleotide sequences (SEQ ID NO: 3); underlining represents similarity between sequences.

FIG. 2 shows a schematic drawing of binary constructs IL24-6, IL24-3, IL24-5, IL24-1, IL24-4, IL24-15, IL24-19, IL24-20, IL24-14, IL24-22, IL24-23, IL24-24, IL24-25, IL24-26 and IL24-27 that may be used for interleukin 24 expression in plants and plant cell culture, for example tobacco BY-2 cell suspension cultures.

FIG. 3A shows a schematic drawing for the cloning strategy for the tobacco-codon optimized synthetic gene containing the 5′UTR, signal peptide, mature IL-24 and tobacco etch virus (TEV) protease site (upper panel). Lower diagram shows a schematic drawing for the cloning strategy for the soluble, modified green fluorescent protein (smGFP) coding region containing the HIS-tag and KDEL motif. FIG. 3B shows a schematic drawing for the cloning strategy for creation of the binary vector pC24-3 used to transform Agrobacterium EHA105 cells for the IL-24:smGFP translational fusion construct targeted to the endoplasmic reticulum (ER) (construct IL24-3).

FIG. 4 shows a schematic drawing for the cloning strategy for creation of the binary vector pC24-6 used to transform Agrobacterium EHA105 cells for the IL-24:smGFP translational fusion construct targeted for secretion (construct IL24-6).

FIG. 5 shows a schematic drawing for the cloning strategy for the tobacco-codon optimized synthetic gene containing the tCUP enhancer, Pr1b signal peptide, mature IL-24 and tobacco etch virus (TEV) protease site (Upper panel). Lower diagram shows a schematic drawing for the cloning strategy for creation of the binary vector pC24-1 used to transform Agrobacterium EHA105 cells for the IL-24:smGFP translational fusion construct targeted to the endoplasmic reticulum (ER) (construct IL24-1).

FIG. 6 shows a schematic drawing for the cloning strategy for creation of the binary vector pC24-5 used to transform Agrobacterium EHA105 cells for IL-24 targeted to the endoplasmic reticulum (ER; construct IL24-5).

FIG. 7 shows a schematic drawing for the cloning strategy for creation of the binary vector pC24-4 used to transform Agrobacterium EHA105 cells for the IL-24:ELP translational fusion construct targeted to the endoplasmic reticulum (ER; construct IL24-4).

FIG. 8 shows a schematic drawing for the cloning strategy for creation of the binary vector pC24-15 used to transform Agrobacterium EHA105 cells for the IL-24::smGFP:ELP translational fusion construct targeted to the endoplasmic reticulum (ER; construct IL24-15).

FIG. 9 shows a schematic drawing for the cloning strategy for creation of the binary vector pC24-19 used to transform Agrobacterium EHA105 cells for the IL-24::ELP translational fusion with native IL-24 signal peptide construct targeted to the endoplasmic reticulum (ER; construct IL24-19).

FIG. 10 shows a schematic drawing for the cloning strategy for the tobacco-codon optimized synthetic gene containing the tCUP enhancer, Prlb signal peptide, aglycosylated IL-24 and tobacco etch virus (TEV) protease site (upper panel). Lower diagram shows a schematic drawing for the cloning strategy (Apa I/Pst I digest) for creation of the binary vector pC24-20 used to transform Agrobacterium EHA105 cells for the IL-24:smGFP:ELP translational fusion construct targeted to the endoplasmic reticulum (ER; construct IL24-20).

FIG. 11A shows a schematic drawing for the cloning strategy for the tCUP enhancer and Rubisco transit peptide (upper panel). Lower diagram shows a schematic drawing for the cloning strategy for the soluble, modified green fluorescent protein (smGFP) coding region containing the HIS-tag and SKL motif. (B) Schematic drawing for the cloning strategy for creation of the binary vector pC24-14 used to transform Agrobacterium EHA105 cells for the IL-24:smGFP translational fusion construct targeted to the chloroplasts and peroxisomes (construct IL24-14).

FIG. 12 shows a schematic drawing for the cloning strategy for creation of the binary vector pC24-22 used to transform Agrobacterium EHA105 cells for the IL-24:smGFP translational fusion construct targeted to the endoplasmic reticulum (ER) under the control of a heat shock inducible promoter (construct IL24-22).

FIG. 13 shows a schematic drawing for the cloning strategy for creation of the binary vector pC24-23 used to transform Agrobacterium EHA105 cells for the IL-24:ELP translational fusion construct targeted to the endoplasmic reticulum (ER) under the control of a heat shock inducible promoter (construct IL24-23).

FIG. 14 shows a schematic drawing for the cloning strategy for creation of the binary vector pRD24-24 used to transform Agrobacterium EHA105 cells for the IL-24:smGFP translational fusion construct targeted to the endoplasmic reticulum (ER) under the control of a heat shock inducible promoter (construct IL24-24).

FIG. 15 shows a schematic drawing for the cloning strategy for creation of the binary vector pRD24-25 used to transform Agrobacterium EHA105 cells for the IL-24:ELP translational fusion construct targeted to the endoplasmic reticulum (ER) under the control of a heat shock inducible promoter (construct IL24-25).

FIG. 16 shows a schematic drawing for the cloning strategy for creation of the binary vector pSRN24-26 used to transform Agrobacterium EHA105 cells for the IL-24:smGFP translational fusion construct targeted to the endoplasmic reticulum (ER) under the control of an ethanol inducible promoter (construct IL24-26).

FIG. 17 shows a schematic drawing for the cloning strategy for creation of the binary vector pSRN24-27 used to transform Agrobacterium EHA105 cells for the IL-24:ELP translational fusion construct targeted to the endoplasmic reticulum (ER) under the control of an ethanol inducible promoter (construct IL24-27).

FIG. 18A shows a screening by quantitative ELISA of total leaf protein for IL-24:smGFP translational fusion protein expression in stable IL24-1 transgenic tobacco plants. FIG. 18B shows the level of expression of IL-24:smGFP translational fusion protein in select high IL-24 expressing stable IL24-1 transgenic tobacco plants over time as measured by quantitative ELISA of total leaf protein. An untransformed 81V9 plant served as a negative control. Letters refer to dates of sampling: A=Dec. 12, 2006; B=Feb. 6, 2007; C=Mar. 8, 2007; D=Jan. 4, 2007; E=Jan. 24, 2007. WP=whole plant, leaves randomly sampled in duplicate; TL=top leaf, leaf near the top, >5 cm in length sampled in duplicate; OL=old leaf, leaf near bottom of plant, but not showing senescence, sampled in duplicate; ML=middle leaf, leaf chosen in between top leaf and old leaf.

FIG. 19A shows a screening by quantitative ELISA of total leaf protein for IL-24:ELP translational fusion protein expression in stable IL24-4 transgenic tobacco plants. FIG. 19B shows the level of expression of IL-24:ELP translational fusion protein in select high IL-24 expressing stable IL24-4 transgenic tobacco plants over time as measured by quantitative ELISA of total leaf protein. An untransformed 81V9 plant served as a negative control. Letters refer to dates of sampling: A=Dec. 14, 2006; B=Jan. 24, 2007; C=Feb. 2, 2007; D=Mar. 8, 2007; E=Jan. 24, 2007; F=Feb. 14, 2007; and G=Dec. 12, 2007. WP=whole plant, leaves randomly sampled in duplicate; TL=top leaf, leaf near the top, >5 cm in length sampled in duplicate; OL=old leaf, leaf near bottom of plant, but not showing senescence, sampled in duplicate; ML=middle leaf, leaf chosen in between top leaf and old leaf.

FIG. 20A shows a screening by quantitative ELISA of total leaf protein for IL-24 protein expression in stable IL24-5 transgenic tobacco plants. FIG. 20B shows the level of expression of IL-24 protein in select high IL-24 expressing stable IL24-5 transgenic tobacco plants over time as measured by quantitative ELISA of total leaf protein. An untransformed 81V9 plant served as a negative control. Letters refer to dates of sampling: A=Feb. 14, 2007; B=Mar. 8, 2007; C=Dec. 12, 2006; D=Feb. 6, 2007. WP=whole plant, leaves randomly sampled in duplicate; TL=top leaf, leaf near the top, >5 cm in length sampled in duplicate; OL=old leaf, leaf near bottom of plant, but not showing senescence, sampled in duplicate; ML=middle leaf, leaf chosen in between top leaf and old leaf.

FIG. 21A shows a western analysis of total leaf protein (50 μg) with biotinylated polyclonal anti-human IL-24 antibody of IL24-1 transformed plants (24-1-27) and IL24-4 transformed plants (24-4-8). TL=top leaf, leaf near the top, >5 cm in length sampled in duplicate. Untransformed 81V9 plant leaf protein (50 μg) was used as a negative control and recombinant human IL-24 protein (25 ng) was used as a positive control. FIG. 21B shows a western analysis of total leaf protein (50 μg) with monoclonal anti-GFP antibody of IL24-1 transformed plants (24-1-27) and alcohol inducible IL24-26 transformed plants (24-26-21), with 10% EtOH induction (+) or without (−). TL=top leaf, leaf near the top, >5 cm in length sampled in duplicate, OL=old leaf, leaf near bottom of plant, but not showing senescence, sampled in duplicate. Untransformed 81V9 plant leaf protein (50 μg) was used as a negative control and recombinant GFP protein (25 ng) was used as a positive control.

FIG. 22A shows a screening by quantitative ELISA of total callus protein for IL-24:smGFP translation fusion protein expression in stable IL24-1 transgenic BY-2 calli. FIG. 22B shows the level of expression of IL-24 protein in high IL-24 expressing stable IL24-1 transgenic BY-2 calli over time as measured by quantitative ELISA of total callus protein. Letters refer to dates of sampling: A=Aug. 16, 2006; B=Nov. 29, 2006; C=Dec. 6, 2006; D=Jan. 16, 2007. FIG. 22C shows the level of expression of IL-24 protein in high IL-24 expressing stable IL24-1 transgenic BY-2 cell suspension cultures over time as measured by quantitative ELISA of total cell protein. D is days after subculturing from D3=day 3 after subculturing to D7=day 7 after subculturing. Letters refer to dates of subculturing: A=Nov. 22, 2006; B=Nov. 29, 2006; C=Dec. 6, 2006; D=Jan. 16, 2007.

FIG. 23A shows a screening by quantitative ELISA of total callus protein for IL-24:smGFP translation fusion protein expression in stable IL24-3 transgenic BY-2 calli. FIG. 23B shows the level of expression of IL-24 protein in high IL-24 expressing stable IL24-3 transgenic BY-2 calli over time as measured by quantitative ELISA of total callus protein. Letters refer to dates of sampling: A=Sep. 22, 2006; B=Nov. 29, 2006; C=Dec. 6, 2006; D=Jan. 16, 2007. FIG. 23C shows the level of expression of IL-24 protein in high IL-24 expressing stable IL24-3 transgenic BY-2 cell suspension cultures over time as measured by quantitative ELISA of total cell protein. D is days after subculturing from D3=day 3 after subculturing to D7=day 7 after subculturing. Letters refer to dates of subculturing: A=Nov. 22, 2006; B=Dec. 6, 2006; C=Jan. 16, 2007.

FIG. 24A shows a screening by quantitative ELISA of total callus protein for IL-24:ELP translation fusion protein expression in stable IL24-4 transgenic BY-2 calli. FIG. 24B shows the level of expression of IL-24 protein in high IL-24 expressing stable IL24-4 transgenic BY-2 calli over time as measured by quantitative ELISA of total callus protein. Letters refer to dates of sampling: A=Aug. 22, 2006; B=Nov. 29, 2006; C=Jan. 16, 2007. FIG. 24C shows the level of expression of IL-24 protein in high IL-24 expressing stable IL24-4 transgenic BY-2 cell suspension cultures over time as measured by quantitative ELISA of total cell protein. D is days after subculturing from D3=day 3 after subculturing to D7=day 7 after subculturing. Letters refer to dates of subculturing: A=Nov. 29, 2006; B=Jan. 16, 2007.

FIG. 25A shows a screening by quantitative ELISA of total callus protein for IL-24 protein expression in stable IL24-5 transgenic BY-2 calli. FIG. 25B shows the level of expression of IL-24 protein in high IL-24 expressing stable IL24-5 transgenic BY-2 calli over time as measured by quantitative ELISA of total callus protein. Letters refer to dates of sampling: A=Aug. 23, 2006; B=Nov. 29, 2006; C=Jan. 16, 2007. FIG. 25C shows the level of expression of IL-24 protein in high IL-24 expressing stable IL24-5 transgenic BY-2 cell suspension cultures over time as measured by quantitative ELISA of total cell protein. D is days after subculturing from D3=day 3 after subculturing to D7=day 7 after subculturing. Letters refer to dates of subculturing: A=Nov. 29, 2006; B=Jan. 16, 2007.

FIG. 26A shows a screening by quantitative ELISA of total callus protein for IL-24:smGFP translation fusion protein expression in stable IL24-6 transgenic BY-2 calli. FIG. 26B shows the level of expression of IL-24 protein in high IL-24 expressing stable IL24-6 transgenic BY-2 calli over time as measured by quantitative ELISA of total callus protein. Letters refer to dates of sampling: A=Sep. 22, 2006; B=Nov. 29, 2006; C=Jan. 16, 2007. FIG. 26C shows the level of expression of IL-24 protein in high IL-24 expressing stable IL24-6 transgenic BY-2 cell suspension cultures over time as measured by quantitative ELISA of total cell protein. D is days after subculturing from D3=day 3 after subculturing to D7=day 7 after subculturing. Letters refer to dates of subculturing: A=Nov. 22, 2006; B=Nov. 29, 2006; C=Jan. 16, 2007.

FIG. 27 show confocal micrographs of an IL-24:smGFP translational fusion protein targeted to the endoplasmic reticulum in transgenic IL24-1 BY-2 cells. FIG. 27A-F shows images taken on Day 3 after subculture; FIG. 27G-L show images taken on Day 6 after subculture. Column 1 (FIGS. 27A, D, G, J)=Green channel; Column 2 (FIGS. 27B, E, H, K)=Transmitted light channel; Column 3 (FIGS. 27C, F, I, L)=Merged channels. (FIGS. 27A-C, 27G-L: Bar=16 μm; FIGS. 27D-F: Bar=7 μm).

FIG. 28A to FIG. 28F show confocal micrographs of an IL-24:smGFP translational fusion construct targeted to the endoplasmic reticulum in transgenic IL24-1 tobacco leaf epidermal cells. Chloroplasts are autofluorescent and detected in the red channel. Column 1 (FIGS. 28A and D)=Green channel; Column 2 (FIGS. 28B and E)=Red channel; Column 3 (FIGS. 28C and F)=Merged channels. (FIGS. 28A-C) Bar=48 μm. (FIGS. 28D-F) Bar=16 μm.

FIG. 29A to FIG. 29F show confocal micrographs of an alcohol inducible IL-24:smGFP translational fusion construct targeted to the endoplasmic reticulum in transgenic IL24-26 tobacco leaf epidermal cells. FIGS. 29A-C show leaves treated with 10% ethanol and left at room temperature for 48 h. FIG. 29D-F show leaves treated with water and left at room temperature for 48 h. Chloroplasts are autofluorescent and detected in the red channel. (Column 1; FIGS. 29A and D) Green channel. (Column 2; FIGS. 29B and E) Red channel. (Column 3; FIGS. 29C and F) Merged channels. Bar=48 μm.

FIG. 30 shows confocal micrographs of an IL-24:smGFP translational fusion construct targeted to the endoplasmic reticulum in transgenic IL24-3 BY-2 cells. FIG. 30A-F shows images taken on Day 3 after subculture; FIG. 30G-I shows images taken on Day 6 after subculture. Column 1 (FIGS. 30A, D, G)=Green channel; Column 2 (FIGS. 30B, E, H)=Transmitted light channel; Column 3 (FIGS. 30C, F, I)=Merged channels. Bar=16 μm.

FIG. 31 shows confocal micrographs of an IL-24:smGFP translational fusion construct targeted for secretion in transgenic IL24-6 BY-2 cells. FIG. 31A-F show images taken on Day 3 after subculture; FIG. 31G-I show images taken on Day 6 after subculture. Column 1 (FIGS. 31A, D, G)=Green channel; Column 2 (FIGS. 31B, E, H)=Transmitted light channel; Column 3 (FIGS. 31C, F, I)=Merged channels. (FIGS. 31A-C) Bar=10 μm; (FIGS. 31D-F) Bar=23 μm; (G-I) Bar=13 μm.

FIG. 32 shows western analysis of purified fractions of IL-24-GFP protein from transgenic leaf material with biotinylated polyclonal anti-human IL-24 antibody. Recombinant human IL-24 protein (25 ng) was used as a positive control. FIG. 32B shows a western analysis of purified IL-24-GFP protein treated with different concentrations of TEV protease with polyclonal anti-human IL-24 antibody. Recombinant human IL-24 protein (25 ng) was used as a positive control. FIG. 32C shows a western analysis of TEV protease cleaved IL-24-GFP protein purified with Ni-NTA agarose beads. First panel uses polyclonal anti-human IL-24 antibody. Second panel uses polyclonal anti-HIS antibody.

FIG. 33 shows SEQ ID NOs:1-149 of the present invention.

### DETAILED DESCRIPTION

The present invention relates to use of plants for the production of interleukin-24 (IL-24) cytokine.

The present invention provides a plant optimized nucleic acid molecule encoding a IL-24 polypeptide or fragment or variant thereof. The present invention also provides a genetic construct comprising a regulatory region operably linked to a plant optimized nucleic acid molecule encoding a IL-24 polypeptide or fragment or variant thereof.

By “plant optimized” it is meant that the IL-24 nucleotide sequence has been optimized for use in a plant. The IL-24 nucleotide sequence may, for example, be a human IL-24 coding sequence that has been analyzed for codon usage and the presence of undesired sequence motifs that could mediate spurious mRNA processing and instability, or methylation of genomic DNA, and produced by ligase chain reaction using synthetic oligonucleotides and a PCR step as described herein in the Examples.

By “operatively linked” it is meant that the particular sequences interact either directly or indirectly to carry out their intended function as described herein. The interaction of operatively linked sequences may for example be mediated by proteins that in turn interact with the sequences. A transcriptional regulatory region and a plant optimized nucleic acid molecule encoding a IL-24 polypeptide or fragment or variant thereof are “operably linked” when the sequences are functionally connected so as to permit transcription of the IL-24 nucleotide sequence to be mediated or modulated by the transcriptional regulatory region.

By “regulatory region” it is meant a nucleic acid sequence that has the property of controlling the expression of a nucleotide sequence that is operably linked with the regulatory region. Such regulatory regions may include promoter or enhancer regions, and other regulatory elements recognized by one of skill in the art. By “promoter” it is meant the nucleotide sequences at the 5′ end of a coding region, or fragment thereof that contain all the signals essential for the initiation of transcription and for the regulation of the rate of transcription and include constitutive promoters, tissue specific promoters or inducible promoters as would be known to those of skill in the art. Examples of known constitutive regulatory elements include but are not limited to promoters associated with the CaMV 35S transcript. (Odell et al., 1985, Nature, 313: 810-812), the rice actin 1 (Zhang et al, 1991, Plant Cell, 3: 1155-1165) and triosephosphate isomerase 1 (Xu et al, 1994, Plant Physiol. 106: 459-467) genes, the maize ubiquitin 1 gene (Cornejo et al, 1993, Plant Mol. Biol. 29: 637-646), the Arabidopsis ubiquitin 1 and 6 genes (Holtorf et al, 1995, Plant Mol. Biol. 29: 637-646), the tobacco translational initiation factor 4A gene (Mandel et al, 1995 Plant Mol. Biol. 29: 995-1004), and the cryptic promoter tCUP (U.S. Pat. No. 5,824,872). The term “constitutive” as used herein does not necessarily indicate that a gene under control of the constitutive regulatory element is expressed at the same level in all cell types, but that the gene is expressed in a wide range of cell types even though variation in abundance is often observed. If tissue specific expression of the gene is desired, for example seed, or leaf specific expression, then promoters specific to these tissues may also be employed.

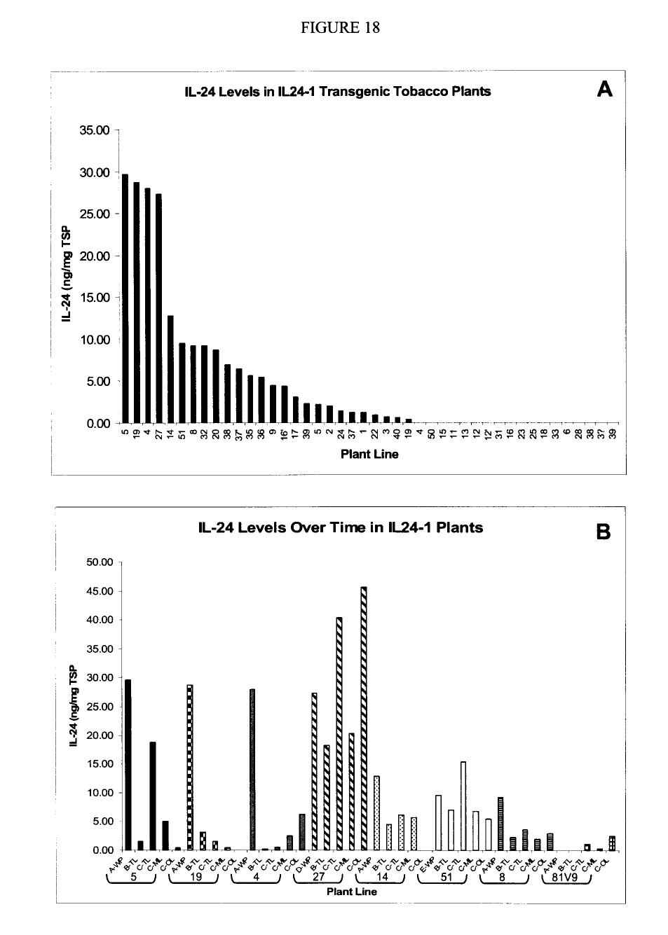

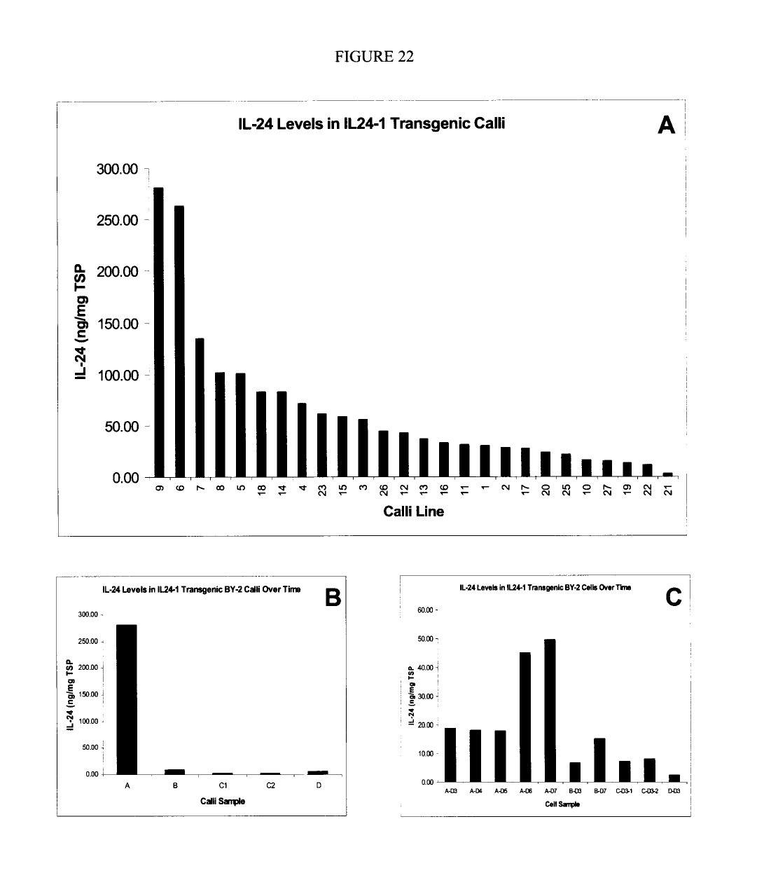

In the Examples disclosed herein, overall, in the majority of the transgenic plants that expressed IL-24, it was found that recombinant IL-24 protein expression decreased with time and the age of the leaf, for example see FIGS. 18B, 19B and 20B. A steady decline in recombinant IL-24 protein expression over time was observed in plant cell culture, for example using tobacco BY-2 calli (see FIGS. 22B, 23B, 24B, 25B, 26B) and suspended transgenic tobacco BY-2 cell cultures (FIGS. 22C, 23C, 24C, 25C, 26C). Without wishing to be bound by theory, it appears that accumulation of recombinant IL-24 in plant tissues and plant cell culture may be toxic to the plant eventually resulting in cell death.

An inducible promoter may be used in order to regulate the expression of the gene following the induction of expression by providing the appropriate stimulus for inducing expression. In the absence of an inducer the nucleic acid sequence will not be transcribed, thereby reducing IL-24 protein accumulation and minimizing the toxic effect of the recombinant IL-24 protein in the plant cell or plant tissue. Therefore, in an embodiment of the present invention the regulatory region comprises an inducible promoter.

The present invention also provides a nucleic acid or a genetic construct comprising an inducible promoter operably linked to a plant optimized nucleic acid molecule encoding a IL-24 polypeptide or fragment or variant thereof. The inducible promoter may be but is not limited to, a heat shock protein (HSP), such as HSP 18.2 (Yoshida, K. et al Appl. Microbiol Biiotechnol. 1995, 44: 466-472; Dansako, T. et al; J. Biosci Bioeng 2003, 95:52-58), HSP 101 (Young et al, Genome 2005, 48:547-555), or an alcohol inducible promoter, for example from Anacyctis nidulans AlcA (Roslan H. A. et al; Plant J 28, 225-235; which is incorporated herein by reference). Other inducible promoters as would be known by those of skill in the art, several of which are described below, may also be used. The inducer can, for example, be a chemical agent such as a protein, metabolite, growth regulator, herbicide or phenolic compound or a physiological stress imposed directly by heat, cold, salt, or toxic elements or indirectly through the action of a pathogen or disease agent such as a virus. A plant cell containing an inducible regulatory element may be exposed to an inducer by externally applying the inducer to the cell or plant such as by spraying, watering, heating or similar methods. Inducible elements may be derived from either plant or non-plant genes (e.g. Gatz, C. and Lenk, I. R. P., 1998, Trends Plant Sci. 3, 352-358; which is incorporated by reference). Examples, of potential inducible promoters include, but not limited to, teracycline-inducible promoter (Gatz, C., 1997, Ann. Rev. Plant Physiol. Plant Mol. Biol. 48, 89-108; which is incorporated by reference), steroid inducible promoter (Aoyama, T. and Chua, N. H., 1997, Plant J. 2, 397-404; which is incorporated by reference), for example but not limited to a dexamethasone-induced promoter, and ethanol-inducible promoter (Salter, M. G., et al, 1998, Plant Journal 16, 127-132; Caddick, M. X., et al, 1998, Nature Biotech. 16, 177-180, which are incorporated by reference) cytokinin inducible IB6 and CKI1 genes (Brandstatter, I. and Kieber, J. J., 1998, Plant Cell 10, 1009-1019; Kakimoto, T., 1996, Science 274, 982-985; which are incorporated by reference) and the auxin inducible element, DR5 (Ulmasov, T., et al., 1997, Plant Cell 9, 1963-1971; which is incorporated by reference), alcohol inducible promoters for example AlcA from Anacycstis nidulans (Roslan H. A. et al; Plant J 28, 225-235; which is incorporated herein by reference). It is also contemplated that the nucleotide sequence of the present invention may also be operatively linked to a wound inducible (Titarenko E., et al., 1997, Plant Physiol. 115:817-826), or pathogenesis related promoter, for example but not limited to a promoter from the gene encoding any of the pathogenesis related proteins PR1-PR15 (van Loon et al., 1999, Phys Mol. Plant Pathol., 55:85-97; Durrant W. E., Dong. X 2004, Annu. Rev. Phytopathol. 42:185-209, which are incorporated herein by reference), including PRI (e.g. Payne G., et al., 1988, Plant Mol. Biol. 11:89-94; Payne G et al., 1989 Plant Mol. Biol. 12:595-596), or a promoter from PRIa-PRIg (van Loon et al., 1999, Phys Mol. Plant Pathol., 55:85-97), PR2 (Ward E. R. 1991, Plant Physiol. 96:390-397), PR3 (Payne G., et al. 1990, PNAS 81:98-102), PR4 (Ward E. R., 1991, Plant Cell, 3:1085-1094), PR5 (Payne G., et al., 1998, Plant Mol. Biol. 11:232-234). Additionally, the nucleotide sequence of the nucleic acid of the present invention may be operatively linked to a promoter encoding a stress-induced protein, resulting from either an abiotic or biotic stress, for example but not limited to a promoter obtained from a jasmonic acid induced gene, for example NPR1 (Kachroo N. A., et al., 2003, Mol. Plant Microbe Interact. 16:588-599). By using a stress, or would-inducible promoter that is operatively linked to the nucleotide sequence of the present invention encoding IL-24, IL-24 may be induced under conditions that result in its selective expression.

In the Examples disclosed herein, a nucleic acids, for example IL24-22 and IL24-23, were prepared comprising an inducible promoter (HSP 18.2) linked to a plant optimized nucleotide sequence encoding IL-24 polypeptide. Construct IL24-22 (SEQ ID NO: 136) included a reporter gene (green fluorescent protein) for identification of transformed plant cells. Construct IL24-23 (SEQ ID NO: 138) included a protein stability gene (elastin-like polypeptide) to increase the level of accumulation of recombinant IL-24.

The present invention therefore further provides a nucleic acid or a genetic construct comprising a nucleotide sequence as set forth in SEQ ID NO: 136 or SEQ ID NO: 138, a nucleotide sequence that exhibits from about 70% to about 100% sequence identity with the nucleotide sequence of SEQ ID NO: 136 or SEQ ID NO: 138, or a nucleotide sequence that hybridizes to the nucleotide sequence of SEQ ID NO: 136 or SEQ ID NO: 138 under conditions of high stringency.

In an embodiment of the present invention, the nucleic acid molecule encoding the plant optimized IL-24 polypeptide or fragment or variant thereof may comprise a nucleotide sequence as set forth in SEQ ID NO: 3. However, the nucleotide sequence may also include a variant that comprises between about 70% to 100% sequence identity, or any amount therebetween with SEQ ID NO:3, for example, but not limited to about 70%, 72%, 75%, 78%, 80%, 82%, 85%, 87%, 90%, 91%, 92%, 93%, 94%, 95%, 96%, 97%, 98%, 99% or about 100% similarity, or any amount therebetween, provided that the fragment or variant thereof when expressed in a plant produces a biologically active IL-24 polypeptide. For example, aglycosylated IL-24 has been shown to retain biological activity (Sauane et al, 2006. Cancer Res. 66(24):11869-77.). Therefore, a nucleotide sequence encoding aglycosylated IL-24 is included within the scope of the present invention. The aglycosylated IL-24 may, for example, have asparagines mutated to alanine as described herein in the Examples (construct IL24-20). The IL-24 polypeptide may comprise an amino acid sequence as set forth in SEQ ID NO: 4.

By “codon optimization” it is meant the selection of appropriate DNA nucleotides for the synthesis of oligonucleotide building blocks, and their subsequent enzymatic assembly, of a structural gene or fragment thereof in order to approach codon usage within plants.

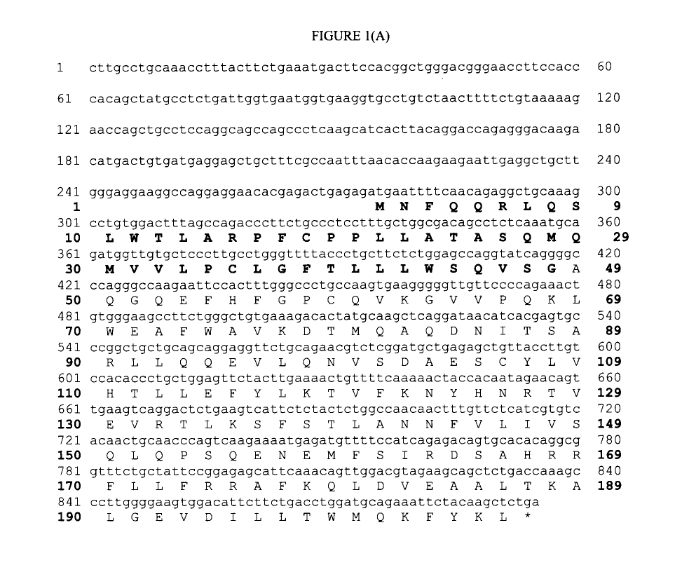

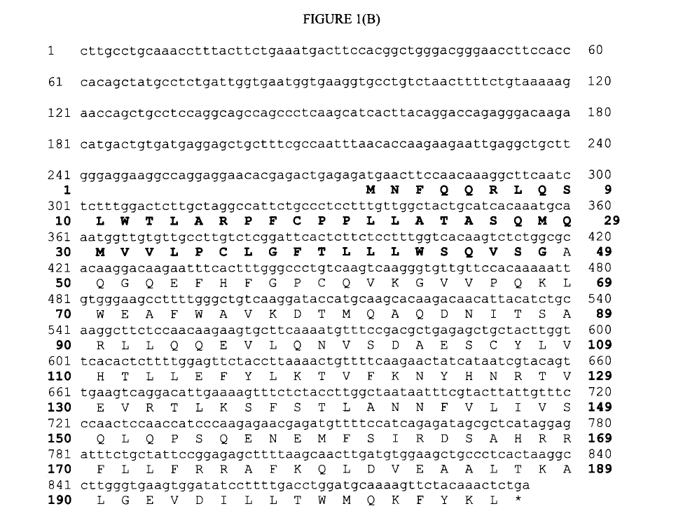

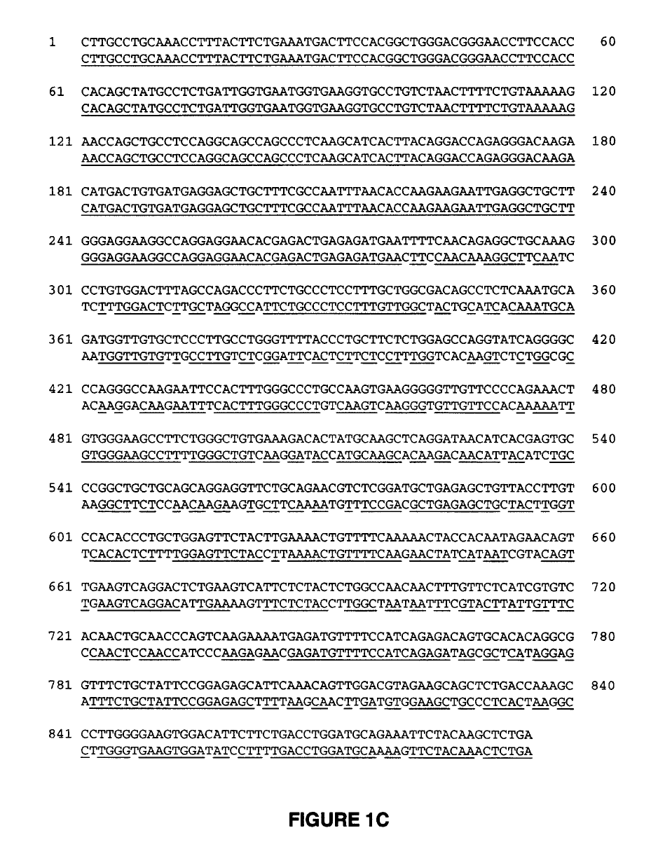

In order to maximize expression levels and protein production of IL-24, the nucleic acid sequence of human IL-24 was examined and the coding region modified to optimize for expression of the gene in plants, for example using a procedure similar to that outlined by Sardana et al. (Plant Cell Reports 15:677-681; 1996). A table of codon usage from highly expressed genes of dicotyledonous plants may be compiled using the data of Murray et al. (Nuc Acids Res. 17:477-498; 1989). An example of a synthetic IL-24 gene comprising codons optimized for expression within plants is shown in FIG. 1B (SEQ ID NO:3). However, it is to be understood that other base pair combinations may be used for the preparation of a plant optimized IL-24 using the methods as described herein in order to optimize expression within a to plant as would be known to one of skill in the art.

To determine whether a nucleic acid molecule exhibits sequence identity with the sequences presented herein, the sequence may be aligned and % identity determined manually, by alignment of the sequences and determining the % identity, or an oligonucleotide alignment algorithm may be used, for example, but not limited to BLAST (Altschul et al., Nuc. Acids Res. 25:3389-3402, 1977; Altschul et al., J. Mol. Biol. 215:403-410, 1990; as available through GenBank; see URL: ncbi.nlm.nih.gov/cgi-bin/BLAST/; using default parameters: Program: blastn; Database: nr; Expect 10; filter: default; Alignment: pairwise; Query genetic Codes: Standard(1)), BLAST2 (EMBL URL: embl-heidelberg.de/Services/index.html using default parameters: Matrix BLOSUM62; Filter: default, echofilter: on, Expect:10, cutoff: default; Strand: both; Descriptions: 50, Alignments: 50), or FASTA (using default parameters). Other similar algorithms may be employed to determine sequence identity between two or more amino acid sequences as would be know to one of skill in the art.

The present invention also includes a nucleotide sequence that hybridizes to SEQ ID NO:3, or a complement of SEQ ID NO:3, under stringent hybridization conditions (see Maniatis et al., in Molecular Cloning (A Laboratory Manual), Cold Spring Harbor Laboratory (1982) p 387 to 389; Ausubel, et al. (eds), 1989, Current Protocols in Molecular Biology, Vol. 1, Green Publishing Associates, Inc., and John Wiley & Sons, Inc., New York, at p. 2.10.3; both of which are incorporated herein by reference).

Without wishing to be limiting in any manner, representative examples of stringent hybridization conditions include hybridization in 4×SSC at 65° C. for 8-16 hours, followed by one, two or three washes in 0.1×SSC at 65° C. for an hour, or hybridization in 5×SSC and 50% formamide at 42° C. for 8 to 16 hours, followed by one, two or three washes in about 0.5×SSC to about 0.2×SSC at 65° C. for one hour. However, hybridization conditions may be modified in accordance with known methods depending on the sequence of interest (see Tijssen, 1993, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, N.Y., which is herein incorporated by reference). Generally, but not wishing to be limiting, stringent conditions are selected to be about 5° C. lower than the thermal melting point for the specific sequence at a defined ionic strength and pH, as can be determined by one of skill in the art.

The nucleic acids or genetic constructs of the present invention can also include enhancers, either translation or transcription enhancers, as may be required. The native 5′-untranslated region (5′UTR) of the plant optimized nucleic acid molecule encoding a IL-24 polypeptide or fragment or variant thereof may be replaced with a translation enhancer, such as but not limited to a tCUP promoter and associated enhancer (see U.S. Pat. No. 5,824,872; U.S. Pat. No. 6,784,289; which are incorporated herein by reference). Alternatively or additionally, the native signal peptide sequence of the plant optimized nucleic acid molecule encoding a IL-24 polypeptide or fragment or variant thereof may be replaced with a heterologous signal/transit peptide. The signal/transit peptide may be a chloroplast targeting peptide, such as but not limited to a ribulose-1,5-bisphosphate carboxylase (Rubisco) transit peptide.

To aid in identification of transformed plant cells or plant cell cultures, the genetic constructs of this invention may be further manipulated to include a reporter gene which expresses a selectable marker or reporter protein. Useful selectable markers include enzymes which provide for resistance to an antibiotic such as gentamycin, hygromycin, kanamycin, and the like. Similarly, enzymes providing for production of a compound identifiable by colour change such as GUS (beta-glucuronidase), or luminescence, such as luciferase are useful. The reporter protein may be green fluorescent protein (GFP), which causes cells that express it to glow green under UV light as described herein in the Examples.

Other elements that may be included in the genetic construct of the present invention comprise sequences for targeting the Il-24 polypeptide to the cytosol or secretory pathway such as, but not limited to, the C-terminal KDEL sequence, an endoplasmic reticulum retention motif (Schouten et al 1966; Menassa et al., 2001 Molecular Breeding, 8: 177-185). Other sequences that may be included in the genetic construct include a peroxisomal targeting signal (PTS), such as but not limited to, the SKL sequence. Other retention signals, as would be known to one of skill in the art, may also be used for this purpose. Furthermore, such a genetic construct may include linker sequences, proteolytic cleavage sequences, and other sequences that aid in the purification of the IL-24 polypeptide. An example, which is not to be considered limiting in any manner, of a sequence that may aid in the purification of the IL-24 polypeptide is an affinity tag, for example, sequences that encode a HIS tag or StrepII. However, it is to be understood that other affinity tags, as are known within the art, may also be used for the purpose of purification. An example, of a sequence that aids in proteolytic cleavage may include, but is not limited to a Tobacco Etch Virus (TEV) protease cleavage sequence. Such a sequence may permit an IL-24 polypeptide to be separated from an attached co-translated sequence, such as, but not limited to a reporter protein sequence, elastin-like polypeptide sequence, or other sequence.

The use of a small ELPs (elastin-like polypeptide) of 9 kDa (Patel et al., 2007 Transgenic Res. 16(2):239-49, incorporated herein by reference), fused with an ER-retained recombinant protein may also be used to increase the accumulation of the recombinant protein. In the

Examples disclosed herein, transformation of tobacco plants with a genetic construct comprising a nucleotide sequence encoding ELP (IL24-4) gave a 10 fold increase in accumulation of recombinant IL-24 compared to plants transformed with a construct without the ELP nucleotide sequence (IL24-1). The genetic construct of the present invention may therefore further comprises a nucleotide sequence encoding an elastin-like polypeptide (ELP) or a fragment thereof.

The present invention therefore provides a nucleic acid comprising:
i) an inducible promoter operably linked to a plant optimized nucleic acid molecule encoding a IL-24 polypeptide or fragment or variant thereof;
ii) a nucleotide sequence encoding an elastin-like polypeptide (ELP) or fragment or variant thereof; and
iii) an endoplasmic reticulum (ER) targeting motif.

The present invention also provides a nucleic acid comprising a nucleotide sequence selected from the group consisting of:
a. SEQ ID NO: 5, SEQ ID NO: 64, SEQ ID NO: 67, SEQ ID NO: 111, SEQ ID NO: 114, SEQ ID NO: 118, SEQ ID NO: 120, SEQ ID NO: 122, SEQ ID NO: 131, SEQ ID NO: 136, SEQ ID NO: 138;
b. a nucleotide sequence that exhibits from about 70% to about 100% sequence identity with the nucleotide sequence of SEQ ID NO: 5, SEQ ID NO: 64, SEQ ID NO: 67, SEQ ID NO: 111, SEQ ID NO: 114, SEQ ID NO: 118, SEQ ID NO: 120, SEQ ID NO: 122, SEQ ID NO: 131, SEQ ID NO: 136, SEQ ID NO: 138; and
c. a nucleotide sequence that hybridizes to the nucleotide sequence of SEQ ID NO: 5, SEQ ID NO: 64, SEQ ID NO: 67, SEQ ID NO: 111, SEQ ID NO: 114, SEQ ID NO: 118, SEQ ID NO: 120, SEQ ID NO: 122, SEQ ID NO: 131, SEQ ID NO: 136, SEQ ID NO: 138 under conditions of high stringency.

The nucleic acid or genetic construct of the present invention may express a fusion protein comprising IL-24 polypeptide or a fragment thereof and a fusion partner. The fusion partner may be a reporter protein, a peptide sequence that may assist in purification, a peptide sequence that assists in expression, the fusion partner may also comprise a peptide sequence that may be used to co-administer with the IL-24. Examples of fusion partners include but are not limited to a reported protein, an elastin-like polypeptide (ELP), a KDEL sequence, a HIS Tag, a pharmaceutically active protein, or a combination thereof.

For example which is not to be considered limiting, the reporter protein may be a green fluorescent protein. The fusion protein may be a polypeptide selected from the group consisting of SEQ ID NO: 6, SEQ ID NO: 65, SEQ ID NO: 68, SEQ ID NO: 112, SEQ ID NO: 115, SEQ ID NO: 119, SEQ ID NO: 121, SEQ ID NO: 123, SEQ ID NO: 132, SEQ ID NO: 137, and SEQ ID NO: 139.

The IL-24 polypeptide described herein may be isolated and produced using standard recombinant and other techniques as described herein or known in the art e.g. see Maniatis et al., in Molecular Cloning (A Laboratory Manual), Cold Spring Harbor Laboratory (1982) p 387 to 389; Ausubel, et al. (eds), 1989, Current Protocols in Molecular Biology, Vol. 1, Green Publishing Associates, Inc., and John Wiley & Sons, Inc., New York, at p. 2.10.3; both of which are incorporated herein by reference).

The present invention provides a plant, or portion thereof, comprising a regulatory region operably linked to a plant optimized nucleic acid molecule encoding a IL-24 polypeptide. The regulatory region may be an inducible promoter, such as but not limited to a heat shock protein (HSP). The plant, or portion thereof, may comprise the genetic construct as hereinbefore described.

The present invention also pertains to a plant cell, comprising a regulatory region operably linked to a plant optimized nucleic acid molecule encoding a IL-24 polypeptide. The present invention also pertains to a plant cell culture, comprising a regulatory region operably linked to a plant optimized nucleic acid molecule encoding a IL-24 polypeptide or fragment or variant thereof. The regulatory region may be an inducible promoter, such as but not limited to a heat shock protein (HSP).

Methods of regenerating whole plants from plant cells are known in the art. In general, transformed plant cells are cultured in an appropriate medium, which may contain selective agents such as antibiotics, where selectable markers are used to facilitate identification of transformed plant cells. Once callus forms, shoot formation can be encouraged by employing the appropriate plant hormones in accordance with known methods and the shoots transferred to rooting medium for regeneration of plants. The plants may then be used to establish repetitive generations, either from seeds or using vegetative propagation techniques.

The genetic constructs of the present invention can be introduced into plant cells using Ti plasmids, Ri plasmids, plant virus vectors, direct DNA transformation, micro-injection, electroporation, etc. For reviews of such techniques see for example Weissbach and Weissbach, Methods for Plant Molecular Biology, Academy Press, New York VIII, pp. 421-463 (1988); Geierson and Corey, Plant Molecular Biology, 2d Ed. (1988); and Miki and Iyer, Fundamentals of Gene Transfer in Plants. In Plant Metabolism, 2d Ed. D T. Dennis, D H Turpin, D D Lefebrve, D B Layzell (eds), Addison Wesly, Langmans Ltd. London, pp. 561-579 (1997). For Arabidospsis see Clough and Bent (1998, Plant J. 16, 735-743). Also considered part of this invention is a vector comprising a genetic construct comprising a regulatory region operably linked to a plant optimized nucleic acid molecule encoding a IL-24 polypeptide or fragment or variant thereof, as hereinbefore described. The vector may be selected from the group consisting of pC24-3, pC24-6-1, pC24-1, pC24-5-1, pC24-4, pC24-15, pC24-19, pC24-20, pC24-14, pRD24-22, pRD24-23, pRD24-24, pRD24-25, pSRN24-26 and pSRN24-27 as disclosed herein.

The present invention also pertains to a plant cell, a transgenic plant, a transgenic plant seed, or a plant cell culture comprising the genetic construct as hereinbefore described, the transgenic plant cell, plant, plant seed, or plant cell culture is characterized as expressing IL-24 polypeptide. The present invention also contemplates a plant, plant cell, plant tissue, plant seed or plant cell culture comprising IL-24 cytokine.

The plant cell, plant cell culture, plant, or portion thereof, may be selected from the group consisting of canola, Brassica spp., maize, tobacco ssp., alfalfa, potato, ginseng, pea, oat, rice, soybean, wheat, barley, sunflower, and cotton. In an embodiment which is not meant to be limiting, the plant cell, cell culture, plant, or portion thereof is a tobacco. In a further aspect of an embodiment, the tobacco is a low-nicotine, low-alkaloid tobacco, such as but not limited to 81V-9.

Also provided in the present invention is a method of producing an IL-24 cytokine for oral administration comprising, transforming a plant, or portion thereof with a regulatory region operably linked to a plant optimized nucleic acid molecule encoding an IL-24 polypeptide or fragment or variant thereof, growing the plant, and expressing the IL-24 cytokine in the plant, or a portion thereof. The regulatory region may be an inducible promoter, such as but not limited to a heat shock protein (HSP).

The present invention also provides a method of producing an IL-24 cytokine for oral administration comprising, transforming a plant, or portion thereof with the genetic construct as hereinbefore described, growing the plant, and expressing the IL-24 cytokine in the plant, or a portion thereof, and harvesting the plant, or a portion thereof comprising IL-24 cytokine for oral administration.

Also according to the present invention, there is provided a method for the production of an IL-24 cytokine, the method comprising,
i) selecting a low nicotine, low alkaloid tobacco plant;
ii) transforming the plant with a vector as hereinbefore described;
iii) growing and harvesting the transformed plant containing the IL-24 polypeptide or fragment or variant thereof, and;
iv) obtaining plant tissues from the transformed plant that comprise the IL-24 cytokine.

There is also provided a method for the production of an IL-24 cytokine comprising,
i) providing a low nicotine, low alkaloid tobacco plant comprising a vector as described herein;
ii) growing and harvesting the low nicotine, low alkaloid tobacco plant containing the IL-24 polypeptide or fragment or variant thereof, and;
iv) obtaining plant tissues from the low nicotine, low alkaloid tobacco plant that comprise the IL-24 cytokine.

Preferably the leaves of the low-nicotine, low-alkaloid tobacco plant comprise, a nicotine concentration (wt/wt) which is less than about 10%, more preferably less than about 5%, still more preferably less than about 2.6% the nicotine concentration (wt/wt) of Delgold tobacco plants grown under the same conditions, and an alkaloid concentration (wt/wt) which is less than about 10%, more preferably less than about 5%, still more preferably less than about 0.28% the alkaloid concentration (wt/wt) of Delgold tobacco plants grown under the same conditions. The alkaloid may consist of, but is not limited to, nicotine, myosmine, anabasine and anatabine.

Also according to the present invention, there is provided a composition comprising plant expressed IL-24, a composition comprising plant-expressed IL-24 and plant matter, for example minimally processed plant-expressed IL-24, a composition comprising partially purified plant-expressed IL-24, or a composition comprising purified plant-expressed IL-24.

Increased mRNA levels of IL-24 and other pro-inflammatory cytokines in MSI-H as compared to MSS, indicate an activated anti-tumour immune response (Banerjea et al., 2004 Mol Cancer. 3(1):21, herein incorporated by reference). These findings suggest that exogenously administered IL-24 improves prognosis for colorectal cancer.

A carcinogen-induced mouse model of colon cancer exhibits pathological and molecular features similar to those in human colon cancer (Corpet and Pierre, 2003 Cancer Epidemiol Biomarkers Prev; 12(5):391-400. Azoxymethane (AOM)-induced mouse tumours are localized primarily to the distal colon and are histologically similar to sporadic forms of human colon cancer (Guda et al., 2004 Oncogene; 23(21):3813-21.). In addition, a number of changes associated with human colorectal cancer have been identified in AOM-induced rodent colon tumours, including alterations in K-ras, adenomatous polyposis coli (APC), cyclin D1, and CDK4, and the presence of microsatellite instability (Nambiar et al., 2002, Corpet and Pierre 2003). Dietary or chemopreventive treatment can be started before exposure to the carcinogen, with the end point being the incidence of colon tumours (Corpet and Pierre 2003). Thus, the mouse model of colon carcinogenesis provides a useful experimental system for studying dietary and preventive agents that could suppress tumours, such as the oral administration of IL-24 in lyophilised tobacco tissue.

The present invention therefore pertains to a method of treating cancer in a subject comprising, orally administering to the subject a composition comprising a plant-expressed IL-24. The cancer may be colorectal cancer.

Further, the present invention provides a method of treating cancer comprising orally administering a plant or portion thereof comprising an IL-24 cytokine to the subject. The cancer may be colorectal cancer.

The present invention pertains to a use a composition comprising a plant-expressed IL-24 for treating cancer in a subject, wherein the composition is orally administrable. The cancer may be colorectal cancer.

Further the present invention pertains to use of a plant or portion thereof comprising a plant-expressed IL-24 for treating cancer in a subject, wherein the plant or portion thereof is orally administrable. The cancer may be colorectal cancer.

Any plant matter comprising material derived from a plant may be administered for treatment. Plant matter may comprise an entire plant, tissue, cells, or a portion, or any fraction, thereof. Further, plant matter may comprise intracellular plant components, extracellular plant components, liquid or solid extracts of plants, or a combination thereof. Further, plant matter may comprise plants, plant cells, tissue, a liquid extract, or a combination thereof, from plant leaves, stems, fruit, roots or a combination thereof. Plant matter may comprise a plant or portion thereof which has not be subjected to any processing steps. However, it is also contemplated that the plant material may be subjected to minimal processing steps as defined below, or more rigorous processing, including partial or substantial protein purification using techniques commonly known within the art including, but not limited to chromatography, electrophoresis and the like.

By the term “minimal processing” it is meant plant matter, for example, a plant or portion thereof comprising a protein of interest which is partially purified to yield a plant extract, homogenate, fraction of plant homogenate or the like. Partial purification may comprise, but is not limited to disrupting plant cellular structures thereby creating a composition comprising soluble plant components, and insoluble plant components which may be separated for example, but not limited to, by centrifugation, filtration or a combination thereof. In this regard, proteins secreted within the extracellular space of leaf or other tissues could be readily obtained using vacuum or centrifugal extraction, or tissues could be extracted under pressure by passage through rollers or grinding or the like to squeeze or liberate the protein free from within the extracellular space. Minimal processing could also involve preparation of crude extracts of soluble proteins, since these preparations would have negligible contamination from secondary plant products. Further, minimal processing may involve methods such as those employed for the preparation of F1P as disclosed in Woodleif et al (1981). These methods include aqueous extraction of soluble protein from green tobacco leaves by precipitation with any suitable salt, for example but not limited to KHSO4. Other methods may include large scale maceration and juice extraction in order to permit the direct use of the extract.

By “oral administration” it is meant the oral delivery of plant matter to a subject in the form of plant material or tissue. The plant matter may be administered as part of a dietary supplement, along with other foods, or encapsulated. The plant matter or tissue may also be concentrated to improve or increase palatability, or provided along with other materials, ingredients, or pharmaceutical excipients, as required.

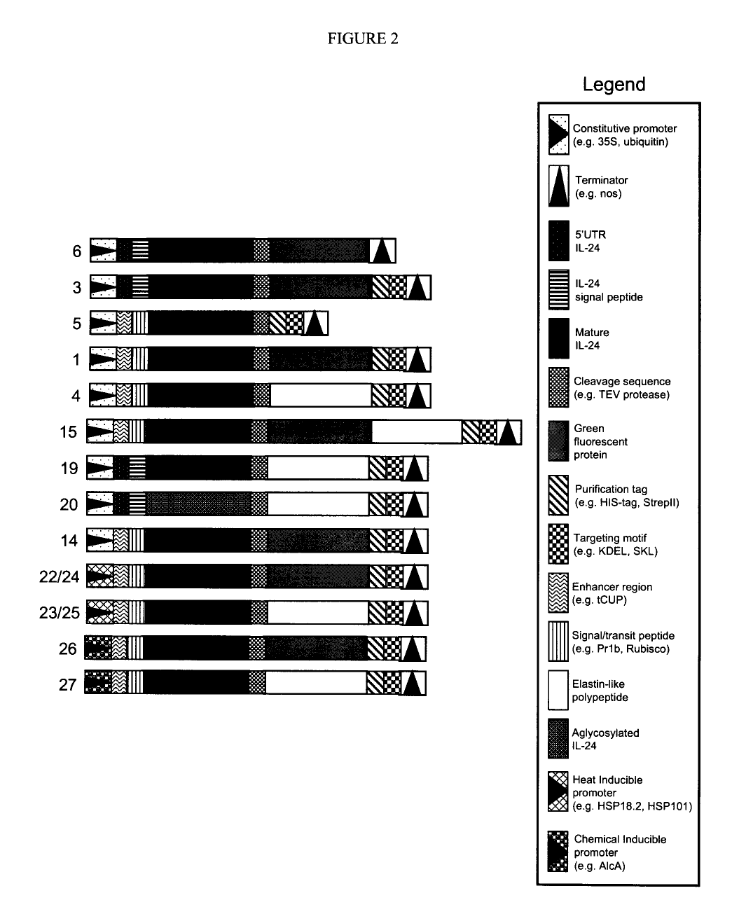

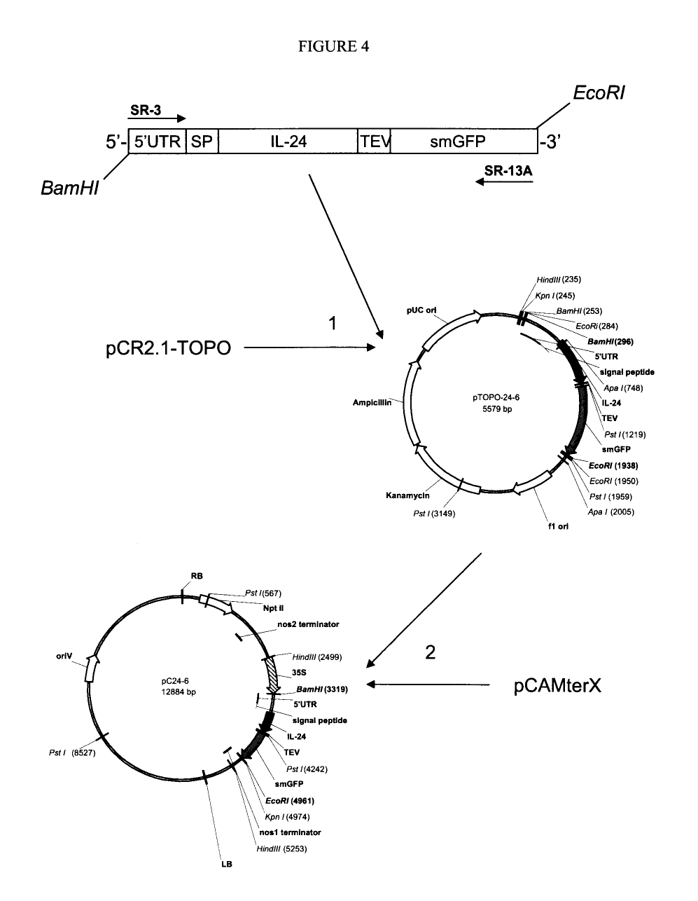

In the Examples disclosed herein, a plant optimized IL-24 gene was produced with its 5′UTR and signal peptide. This protein is secreted to the apoplast in leaves or the extracellular medium in cell suspension culture for ease of purification. Construct IL24-6 (SEQ ID NO:64) is a clone designed to express in a constitutive manner which is constructed as per FIG. 4. As seen in FIG. 2, the IL24-6 clone consists of constitutive promoter (CaMV 35S) and terminator (nos) driving the expression of a fusion protein (SEQ ID NO:65) between IL-24 and soluble modified green fluorescent protein (smGFP).

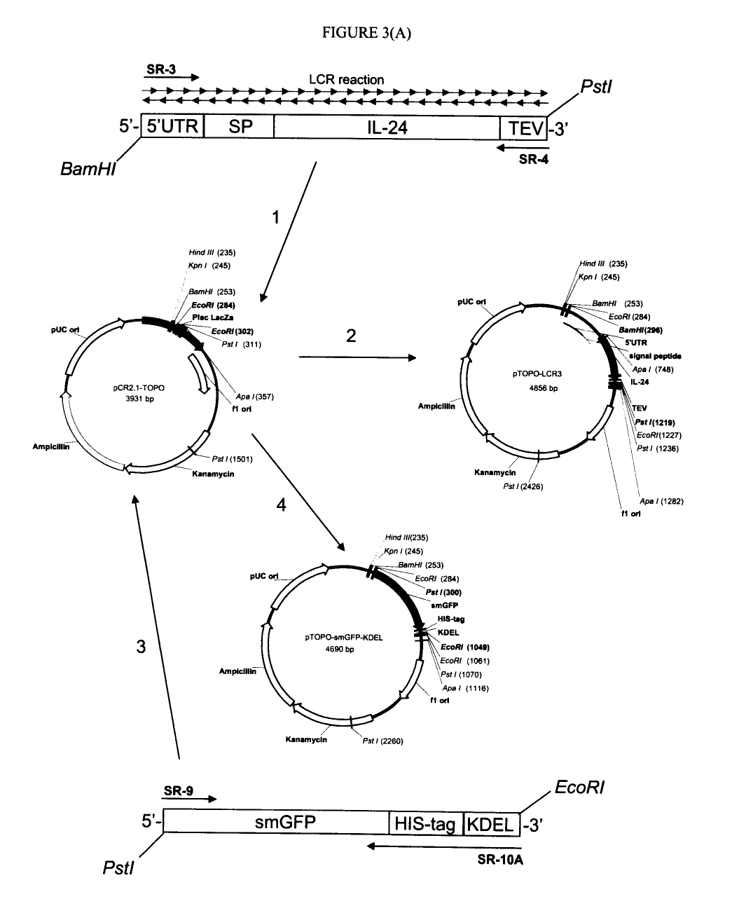

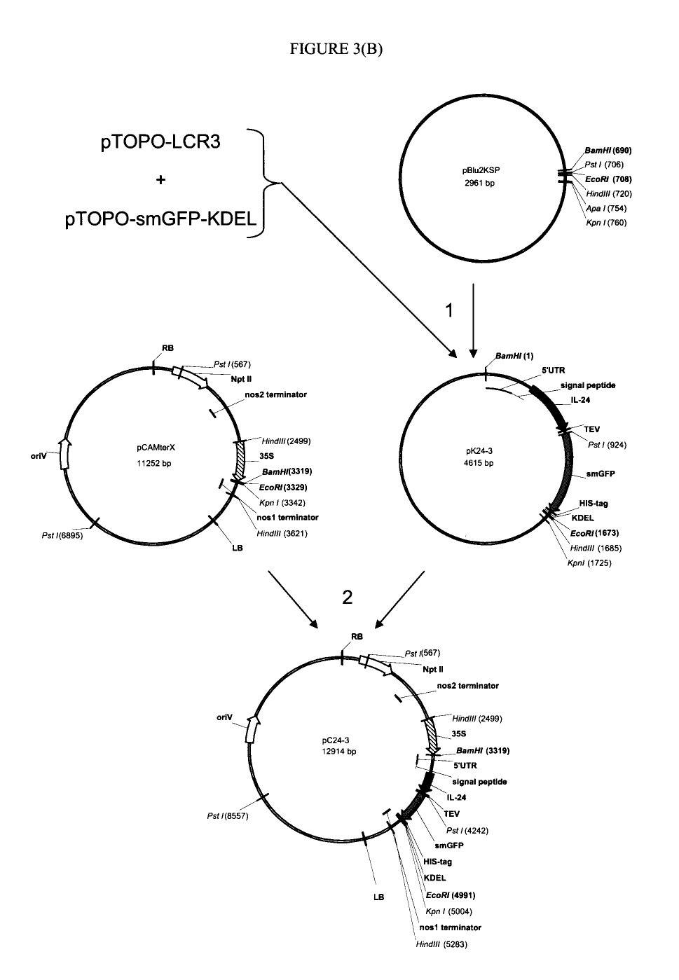

Construct IL24-3 (SEQ ID NO:5) is a clone designed to express in a constitutive manner which is constructed as per FIGS. 3(A) and 3(B). As seen in FIG. 2, the IL24-3 clone consists of constitutive promoter (CaMV 35S) and terminator (nos) driving the expression of a fusion protein (SEQ ID NO:6) between IL-24 and smGFP with an ER retrieval motif (KDEL).

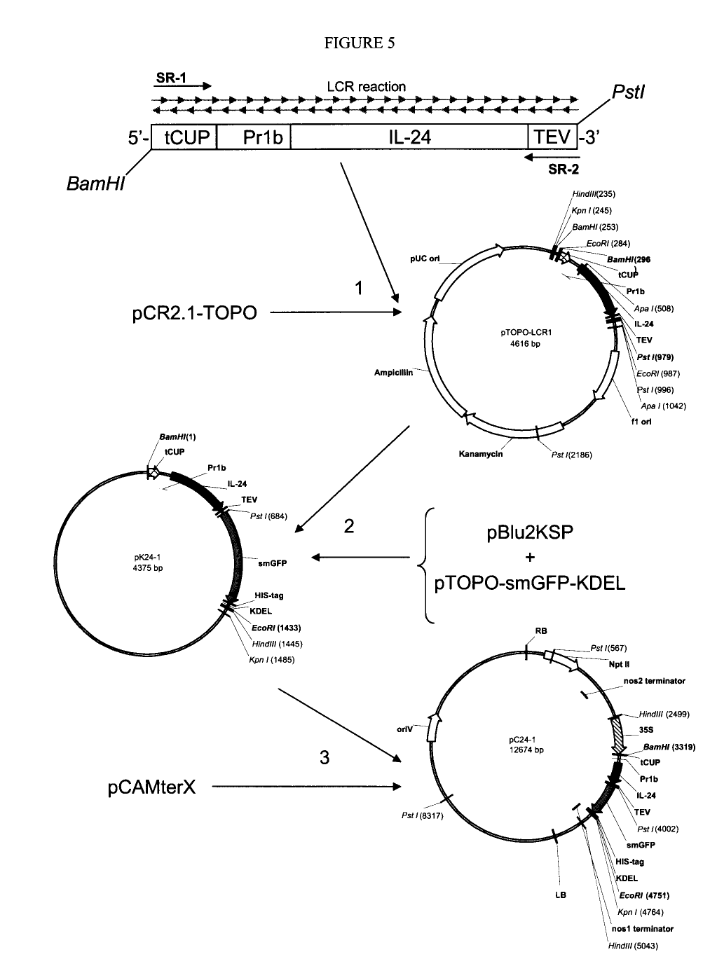

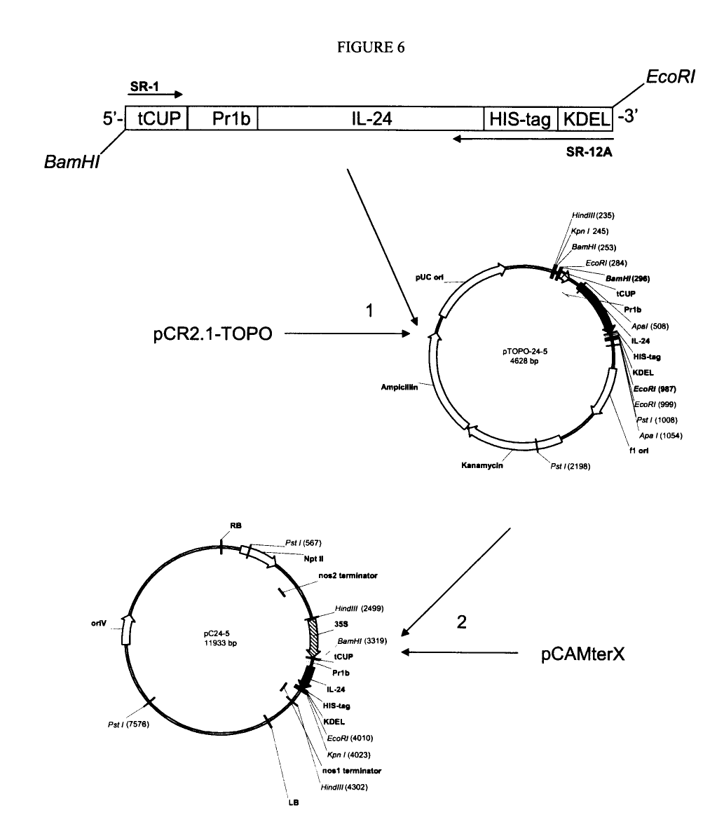

To maximize potential IL-24 production at the translational level, the native 5′-untranslated region of IL-24 cDNA may be replaced with a desired promoter or regulatory region, for example the translation enhancer of the tCUP promoter (also known as T1275; U.S. Pat. No. 5,824,872, which is incorporated herein by reference). The native signal peptide sequence may also be replaced with a desired signal sequence, for example, the signal sequence of the tobacco pathogenesis-related protein 1b (PR-1b) (Cornelissen et al 1986, Sijmons et al 1990, Denecke et al 1990). In the examples provided below, several clones were created, one with a translational fusion to smGFP for subcellular localization (IL24-1), and another without the reporter protein (IL24-5). Construct IL24-1 (SEQ ID NO:67; see FIG. 5) is a clone designed to express in a constitutive manner. As seen in FIG. 2, the IL24-1 clone consists of constitutive promoter (CaMV 35S) and terminator (nos) driving the expression of a fusion protein (SEQ ID NO:68) between IL-24 and soluble modified green fluorescent protein (smGFP) with an ER retrieval motif (KDEL). Construct IL24-5 (SEQ ID NO:111; see FIG. 6) is a clone designed to express in a constitutive manner. As seen in FIG. 2, the IL24-5 clone consists of constitutive promoter (CaMV 35S) and terminator (nos) driving the expression of a IL-24 protein (SEQ ID NO:112) with an ER-retrieval motif (KDEL).

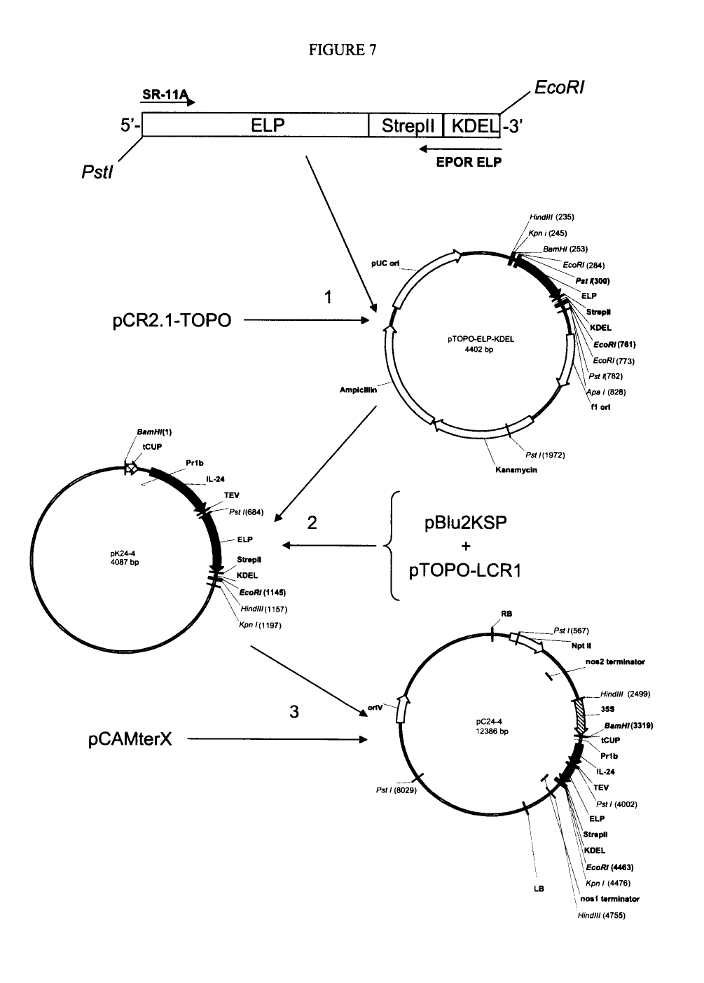

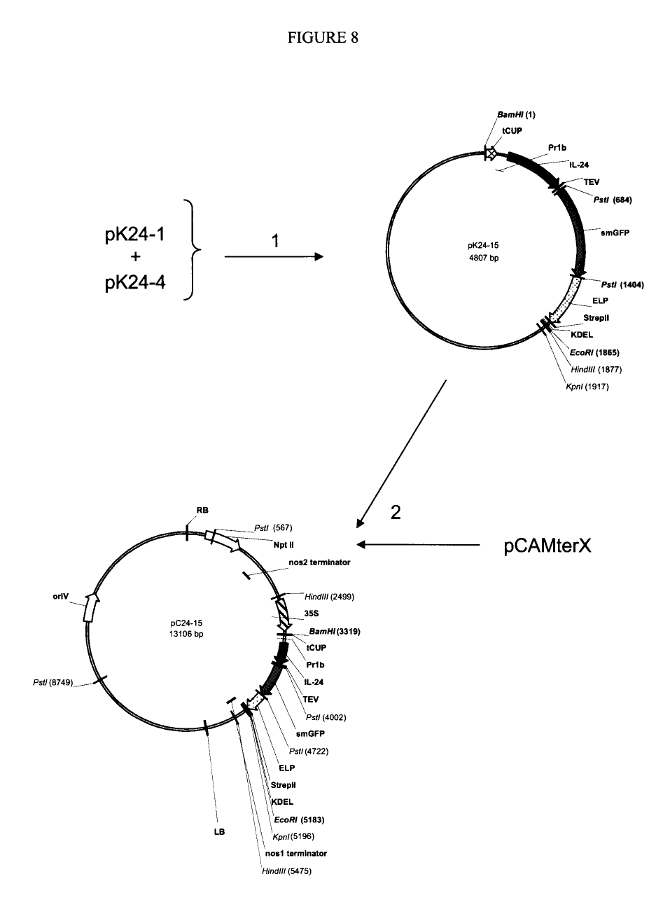

Construct (IL24-4) was produced for expression of a fusion protein between IL-24 and an ELPs (elastin-like polypeptide; Patel et al., 2007). Construct IL24-4 (SEQ ID NO:114; see FIG. 7) is a clone designed to express in a constitutive manner. As seen in FIG. 2, the IL24-4 clone consists of constitutive promoter (CaMV 35S) and terminator (nos) driving the expression of a fusion protein (SEQ ID NO:115) between IL-24 and an elastin-like polypeptide (ELP) with an ER retrieval motif (KDEL). To visualize the subcellular localization, another construct was produced (IL24-15) that inserted smGFP between TEV and ELP. Construct IL24-15 (SEQ ID NO:118) is a clone designed to express in a constitutive manner which is constructed as per FIG. 8. As seen in FIG. 2, the IL24-15 clone consists of constitutive promoter (CaMV 35S) and terminator (nos) driving the expression of a fusion protein (SEQ ID NO:119) between IL-24, smGFP and an elastin-like polypeptide (ELP) with an ER retrieval motif (KDEL).

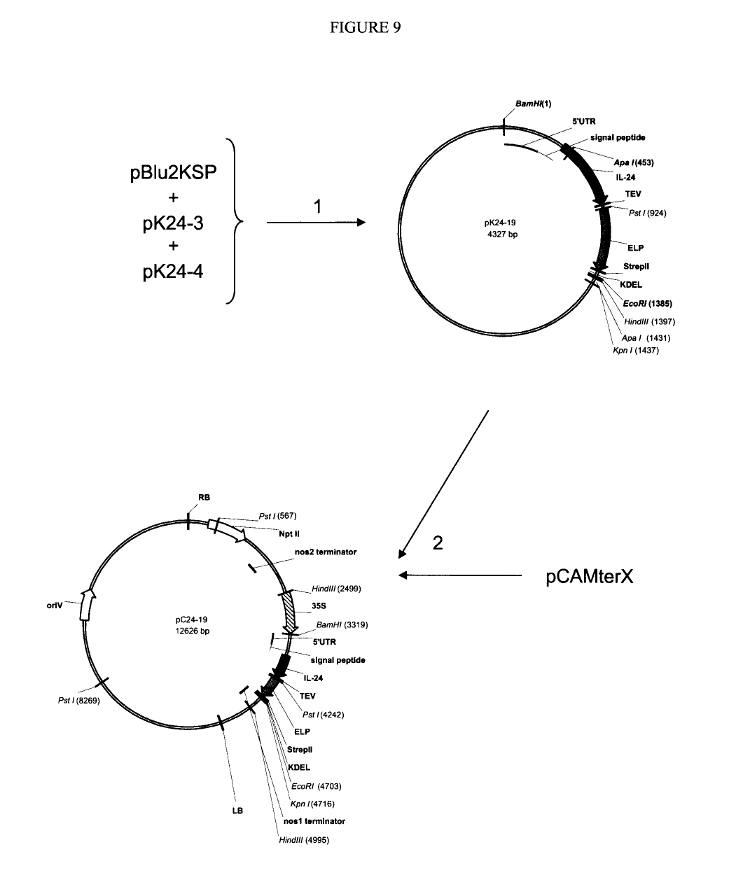

The 5′UTR and signal peptide were observed to produce increased IL-24 levels than the tCUP-PR1b. Similarly, the inclusion of the ELP sequence increased accumulation levels considerably. A construct (IL24-19) was created to include these elements. Construct IL24-19 (SEQ ID NO:120) is a clone designed to express in a constitutive manner which is constructed as per FIG. 9. As seen in FIG. 2, the IL24-19 clone consists of constitutive promoter (CaMV 35S) and terminator (nos) driving the expression of a fusion protein (SEQ ID NO:121) between IL-24 and an elastin-like polypeptide (ELP) with an ER retrieval motif (KDEL).

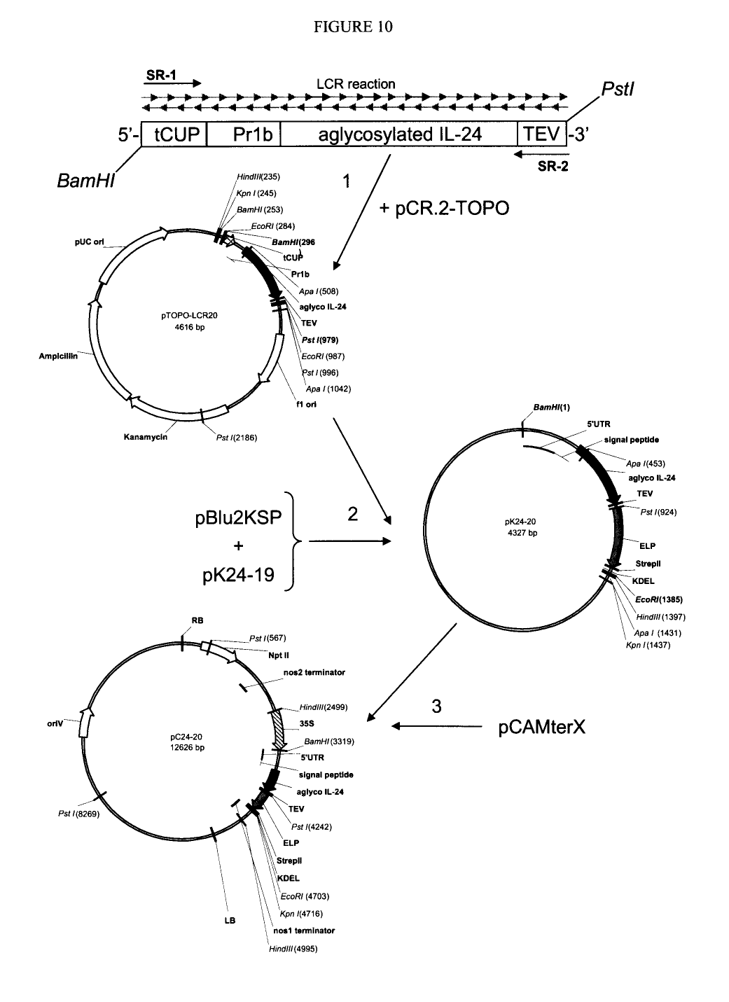

It is known that IL-24 retains biological activity even when it is not glycosylated (Sauane et al, 2006). Thus, an aglycosylated IL-24 construct was produced (IL24-20) where three asparagines were mutated to alanine. Construct IL24-20 (SEQ ID NO:122) is a clone designed to express in a constitutive manner which is constructed as per FIG. 10. As seen in FIG. 2, the IL24-20 clone consists of constitutive promoter (CaMV 35S) and terminator (nos) driving the expression of a fusion protein (SEQ ID NO:123) between an aglycosylated IL-24 and an elastin-like polypeptide (ELP) with an ER retrieval motif (KDEL).

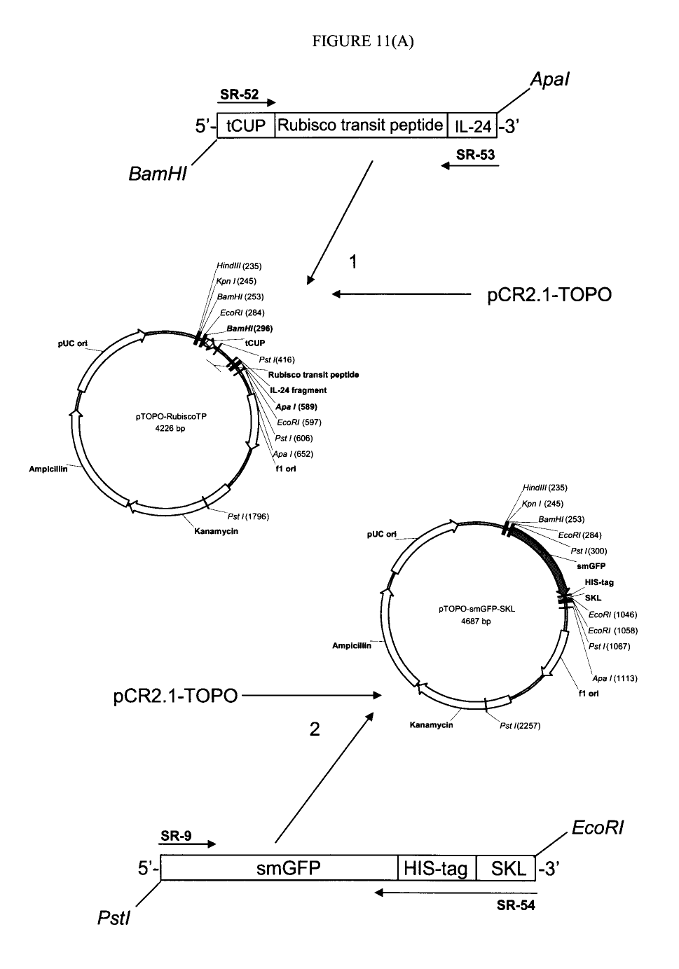

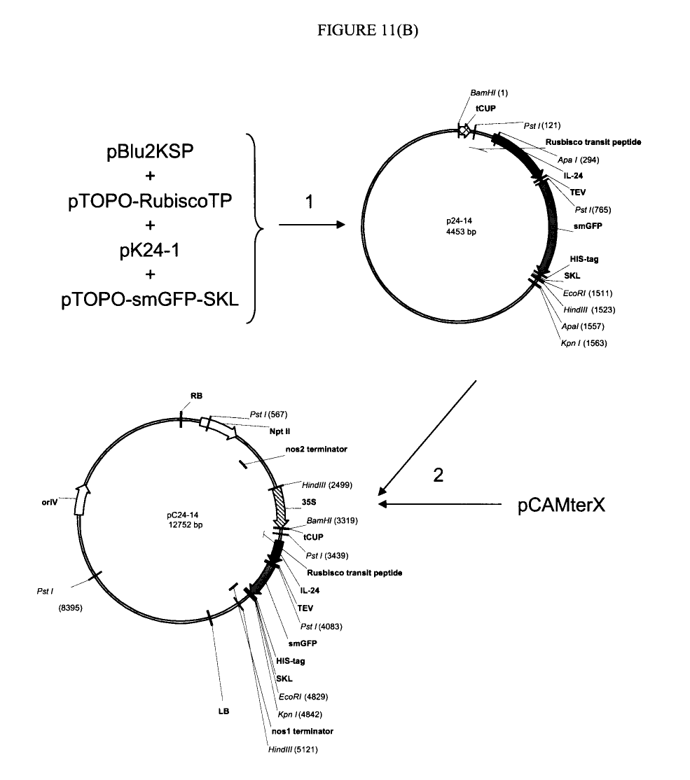

Some proteins accumulate to higher levels in the chloroplast, and thus a construct (IL24-14) was designed to target IL-24 to both the chloroplast and the peroxisome. Construct IL24-14 (SEQ ID NO:131) is a clone designed to express in a constitutive manner which is constructed as per FIGS. 11A and B. As seen in FIG. 2, the IL24-14 clone consists of constitutive promoter (CaMV 35S) and terminator (nos) driving the expression of a fusion protein (SEQ ID NO:132) between IL-24 and smGFP targeted to the chloroplasts using a transit peptide and to the peroxisomes using a SKL motif.

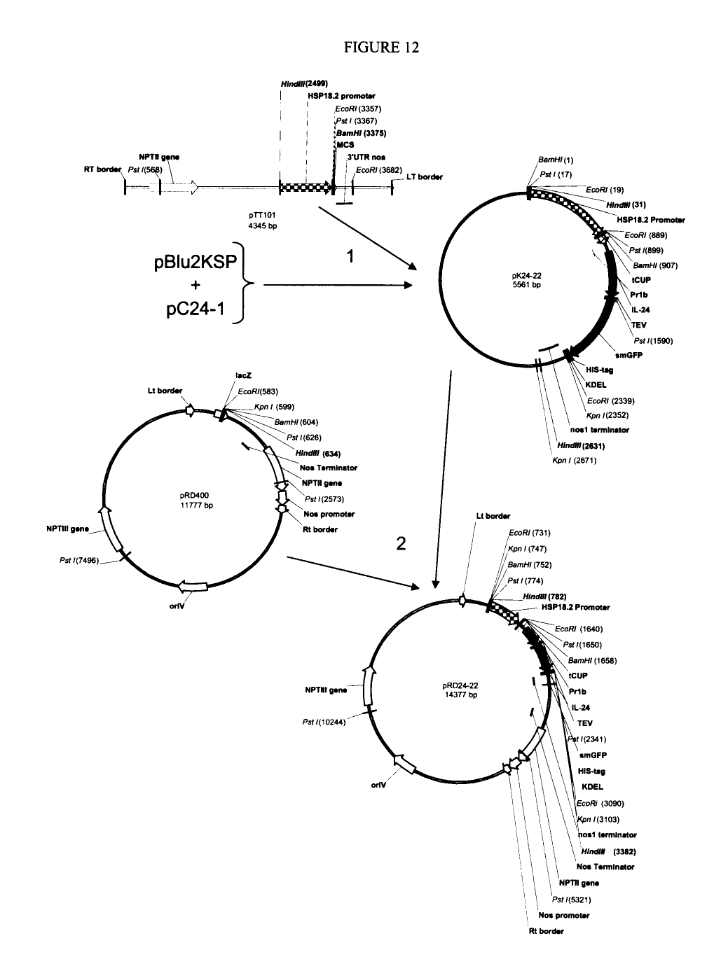

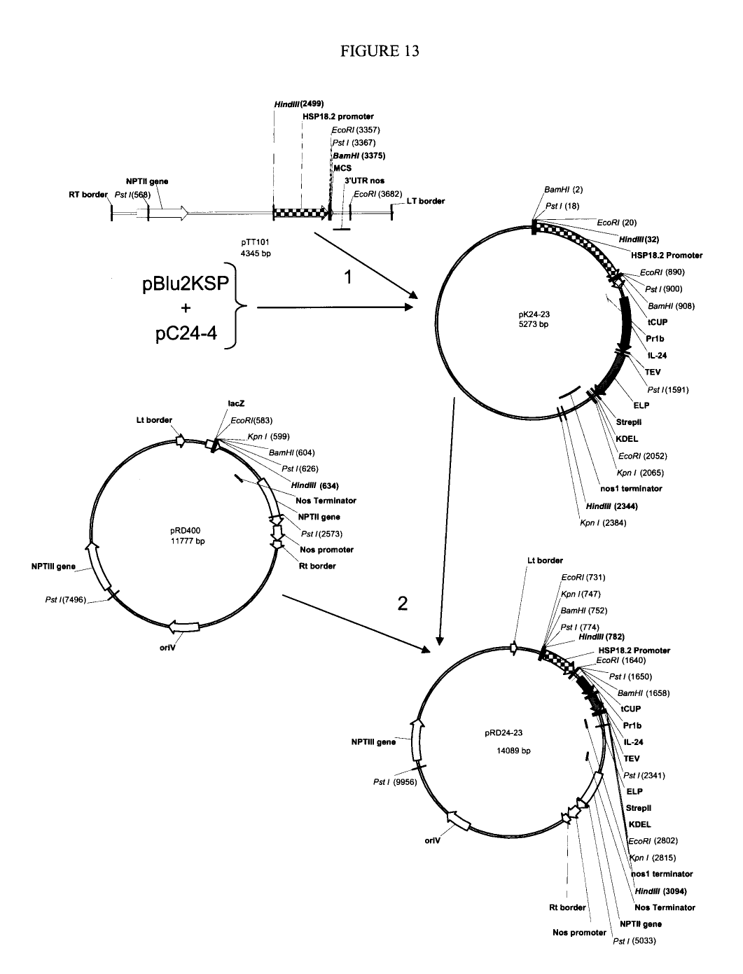

The use of an inducible promoter for inducing expression in cell cultures (for example but not limited to, HSP 18.2), and for induction in plants, for example but not limited to tobacco plants, was examined. Two constructs were created, one with the reporter gene, smGFP (IL24-22) and the other with protein stability gene, ELP (IL24-23). Construct IL24-22 (SEQ ID NO:136) is a clone designed to express in a heat shock inducible manner which is constructed as per FIG. 12. As seen in FIG. 2, the IL24-22 clone consists of inducible promoter (HSP 18.2) and terminator (nos) driving the expression of a fusion protein (SEQ ID NO:137) between IL-24 and smGFP with an ER retrieval motif (KDEL). Construct IL24-23 (SEQ ID NO:138) is a clone designed to express in a heat shock inducible manner which is constructed as per FIG. 13. As seen in FIG. 2, the IL24-23 clone consists of inducible promoter (HSP 18.2) and terminator (nos) driving the expression of a fusion protein (SEQ ID NO:139) between IL-24 and ELP with an ER retrieval motif (KDEL).

The expression systems adopted a duplicated 35S enhancer-promoter plus tCUP leader sequence, however other constitutive promoters and 5′UTRs may also be employed, for example but not limited to, T1276 a constitutive promoter from tobacco (also referred to as tCUP; U.S. Pat. No. 5,824,872; which is incorporated herein by reference), a heat inducible promoter (e.g. HSP 18.2), alcohol inducible promomter (e.g. AlcA), or a methyl jasmonate inducible promoter from the soybean vegetative protein.

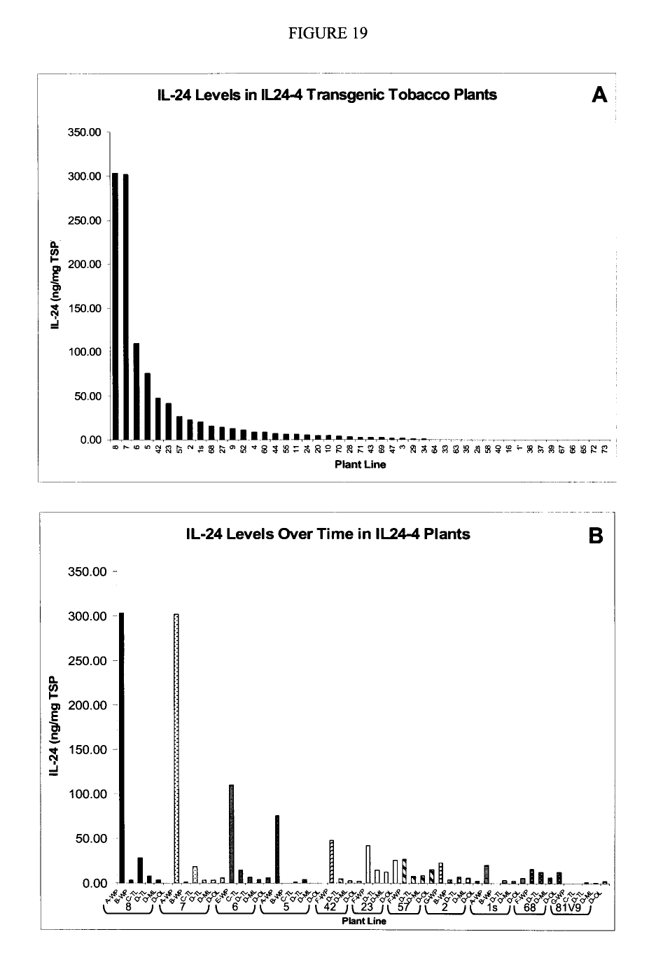

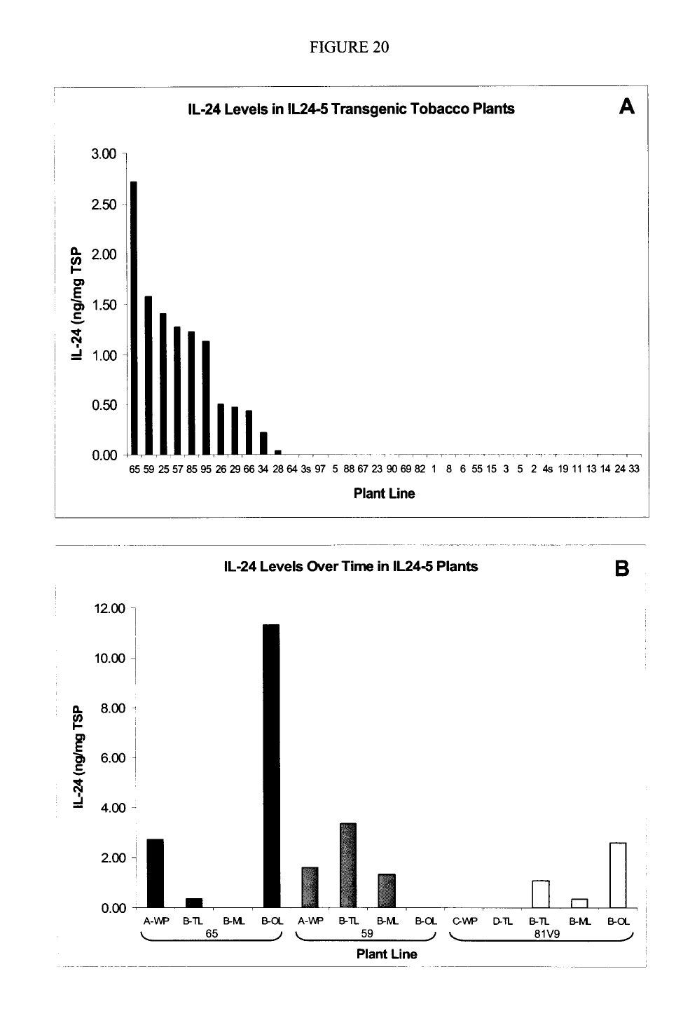

A variable amount of plant recombinant IL-24 (prIL-24) was observed in IL24-1, IL24-4 and IL24-5 transgenic tobacco plants (FIG. 18A, FIG. 19A, FIG. 20A). However, the highest level of prIL-24 was obtained with construct IL24-4 (tCUP-Pr1b::IL-24::TEV::ELP::Strepll::KDEL; FIG. 19A). Very little IL-24 protein expression was detected with construct IL24-5 (tCUP-Pr1b::IL-24::His::KDEL; FIG. 20A). Construct IL24-1 including a nucleotide sequence encoding reporter protein smGFP (tCUP-Pr1b::IL-24::smGFP::His::KDEL) (FIG. 18A) showed higher IL-24 protein expression than construct IL24-5 without the reporter protein sequence. Overall, in the majority of the IL24 transgenic tobacco plants, IL-24 protein expression decreased with time and the age of the leaf (FIG. 18B, FIG. 19B, FIG. 20B).

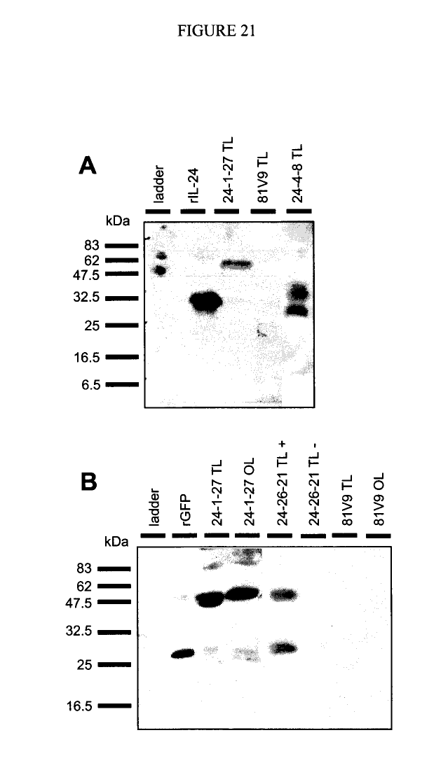

Western analysis of total leaf protein using biotinylated polyclonal anti-human IL-24 antibody of IL24-1 transformed plants (24-1-27) and IL24-4 transformed plants (24-4-8) is shown in FIG. 21A, and using monoclonal anti-GFP antibody of IL24-1 transformed plants (24-1-27) and alcohol inducible IL24-26 transformed plants (24-26-21), with 10% EtOH induction (+) or without (−), in FIG. 21B. The results show that pIL-24 is correctly produced in plants, and that IL24 may be induced in IL24-26 plants in the presence of an inducer (compare lanes 24-26-21 TL+ (in the presence of inducer, ethanol) and 24-26-21 TL− (no inducer).

Of interest, is that the IL24-26 plants (comprising IL24 under the control of A1cA inducible promoter) are healthy, and no loss in expression is observed over time since the expression of IL24 is selectively controlled. Since IL24 is not expressed in the IL24-26 plants in the absence of the inducer, these plants are healthier when compared to plants expressing IL24 constitutively. The IL24-26 plants, or other plants comprising IL24 operabely linked to an inducible promoter, can be grown to any desired stage of development, or age, prior to inducing expression of IL24. In this was, the yield of plant expressed IL-24 can be optimized, and since more plant mass is harvested from healthier transgenic plants, increased yields of IL24 may be obtained.

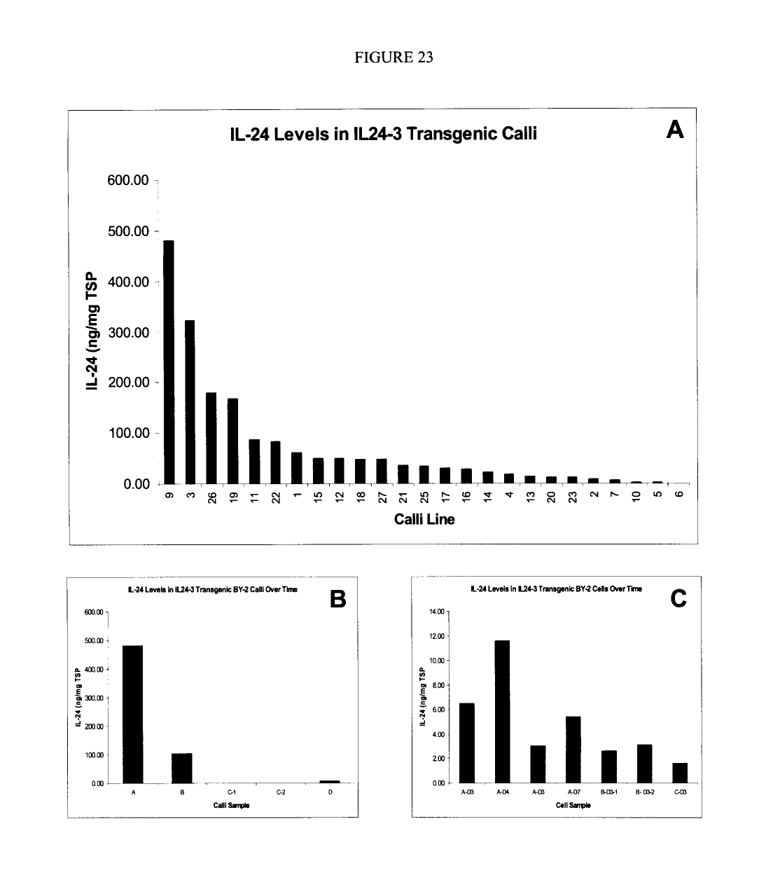

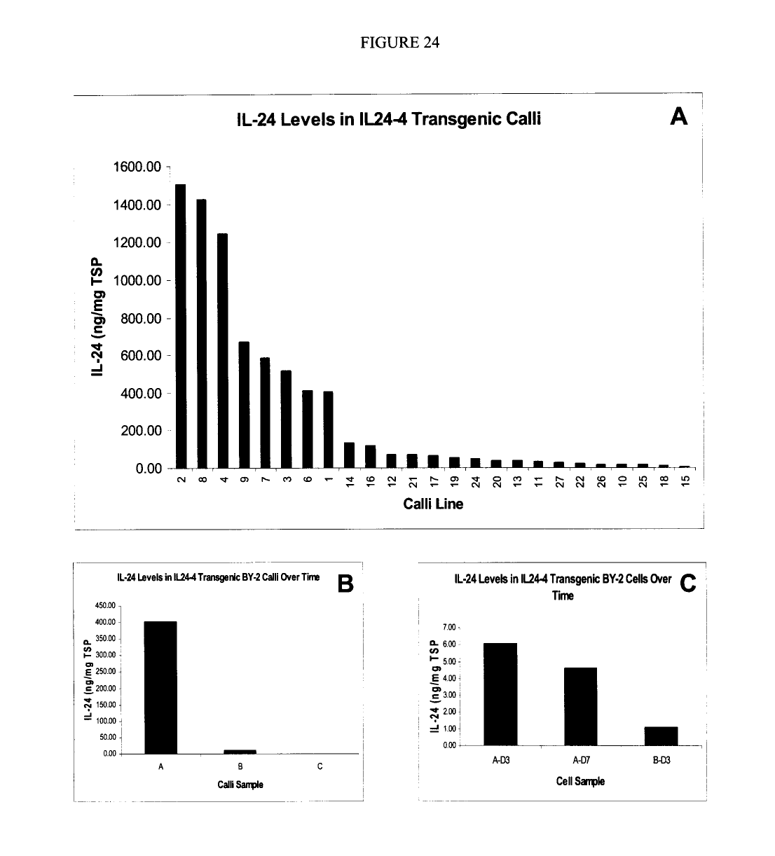

A variable amount of plant recombinant IL-24 (prIL-24) is observed in IL24-1, IL24-3, IL24-4, IL24-5 and IL24-6 transgenic BY-2 calli (FIGS. 22A, 23A, 24A, 25A, 26A). The highest level of prIL-24 is obtained with construct IL24-4 (tCUP-Pr1b::IL-24 TEV ELP StrepII KDEL; FIG. 24A). A comparison of the native IL-24 5′UTR and signal peptide (construct IL24-3, FIG. 23A) versus the tCUP enhancer and Pr1b signal peptide (construct IL24-1, FIG. 22A) suggests that the native 5′UTR and signal peptide promote higher levels of IL-24 expression in BY-2 transgenic calli. Overall, a steady decline in recombinant IL-24 protein expression over time was observed in transgenic tobacco BY-2 calli (FIGS. 22B, 23B, 24B, 25B, 26B) and suspended transgenic tobacco BY-2 cell culture (FIGS. 22C, 23C, 24C, 25C, 26C).

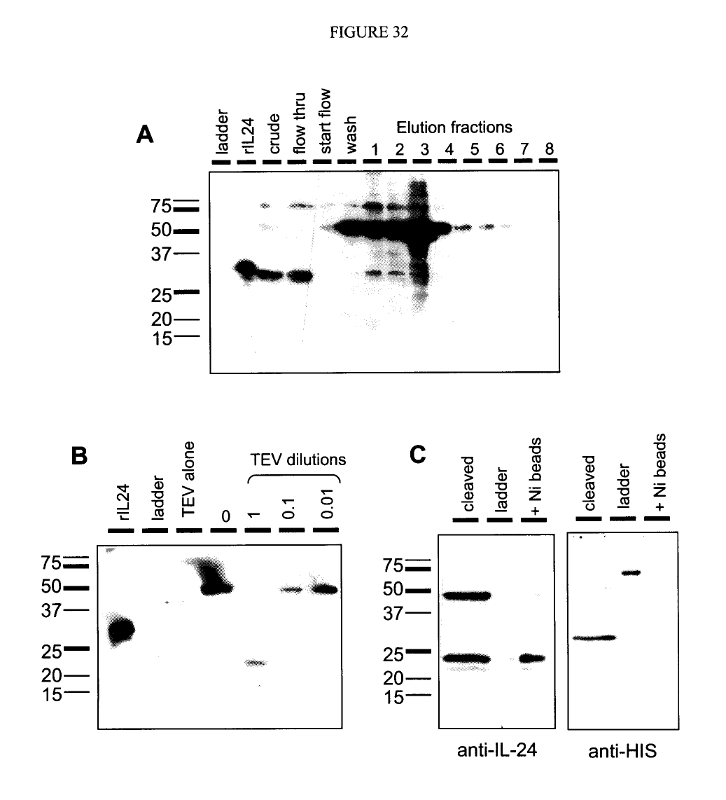

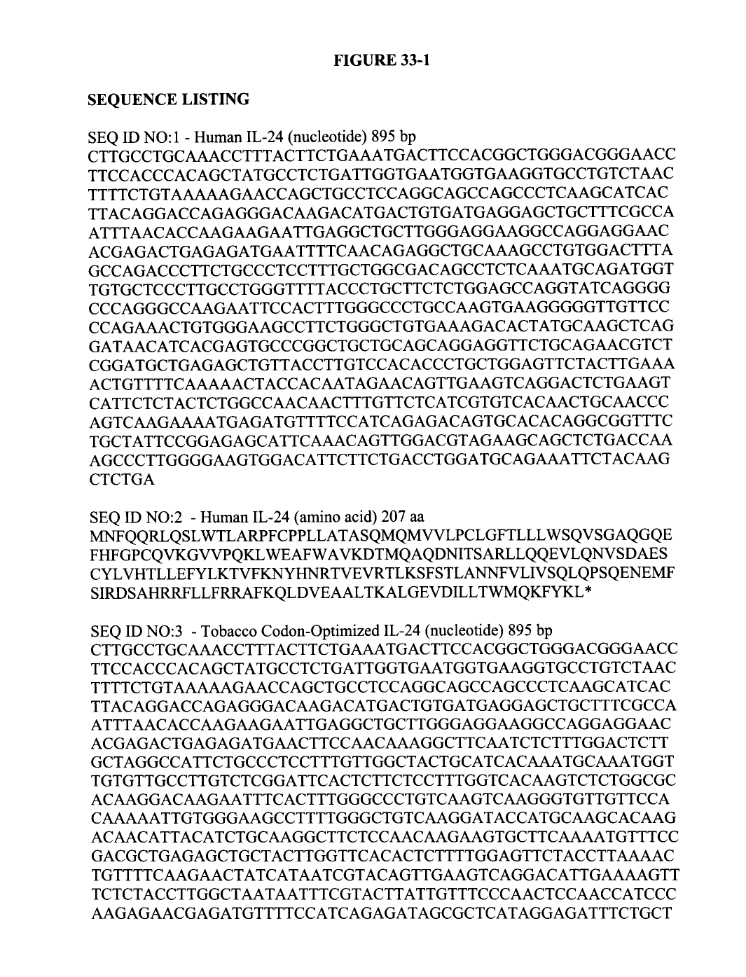

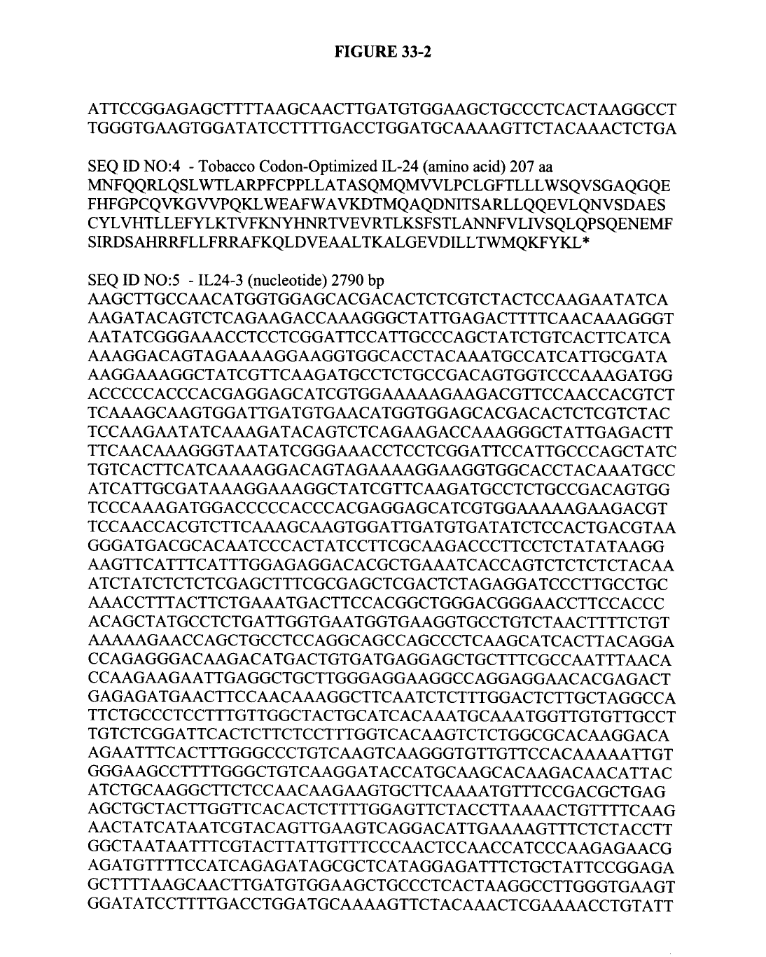

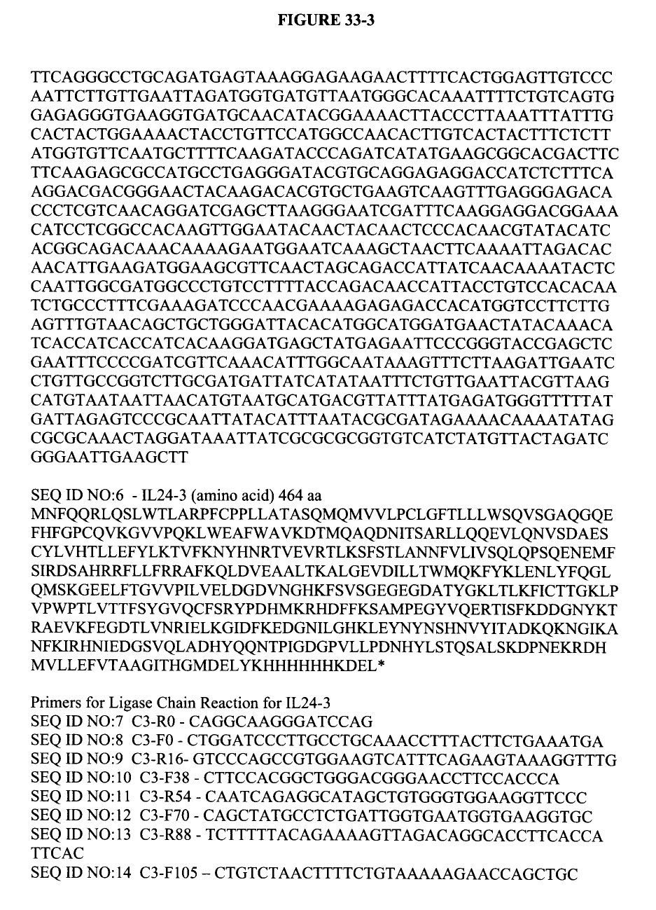

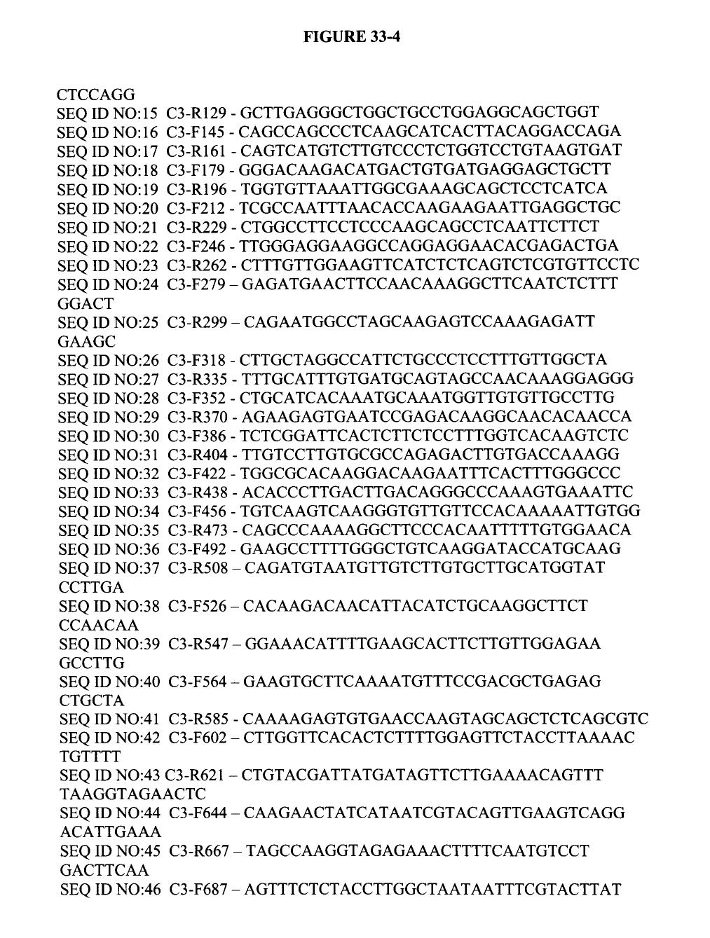

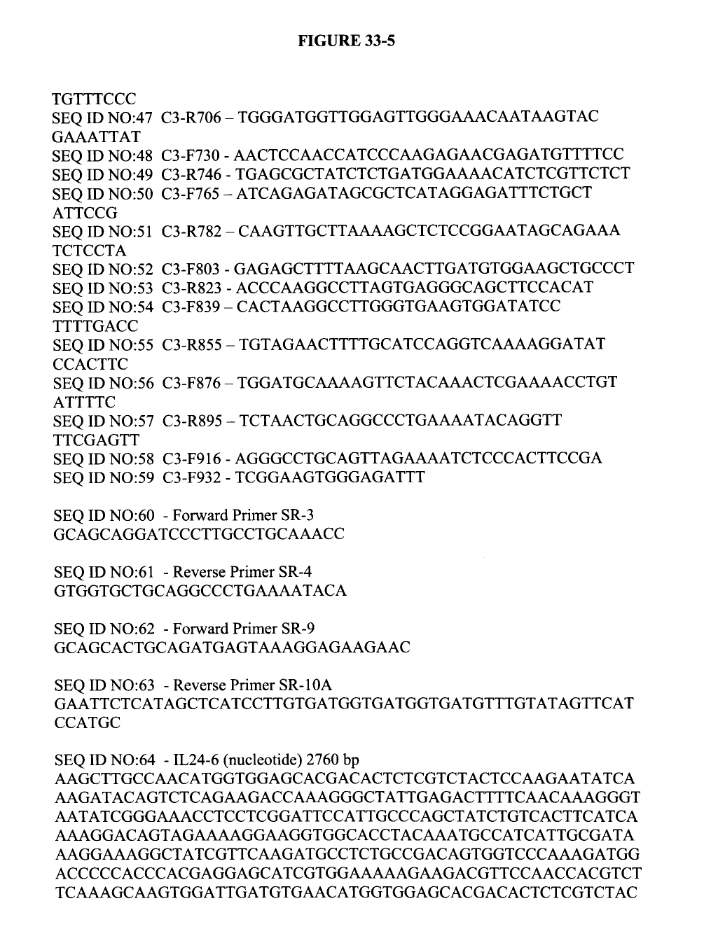

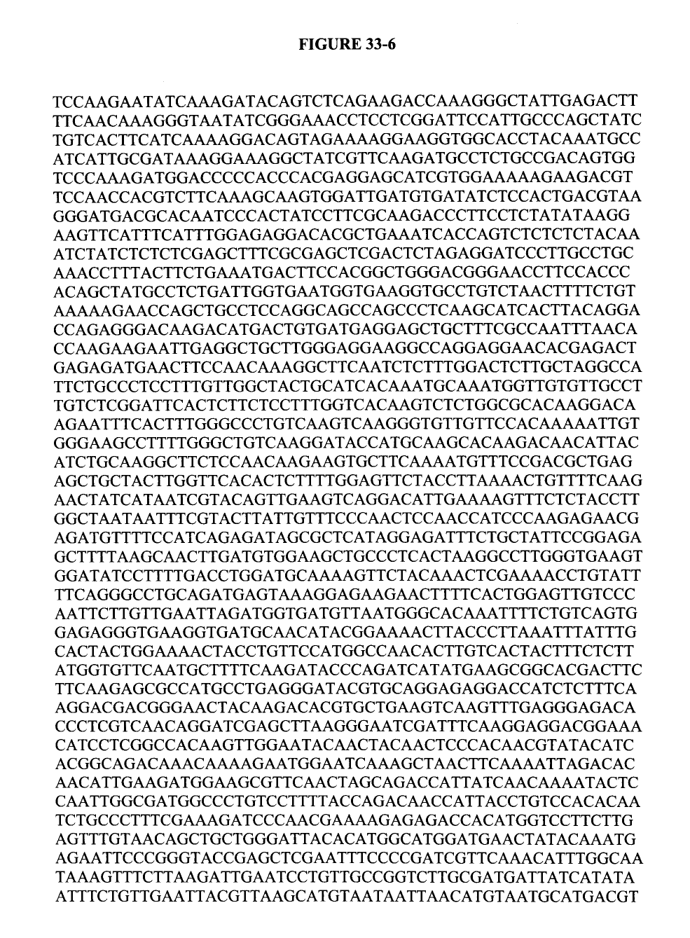

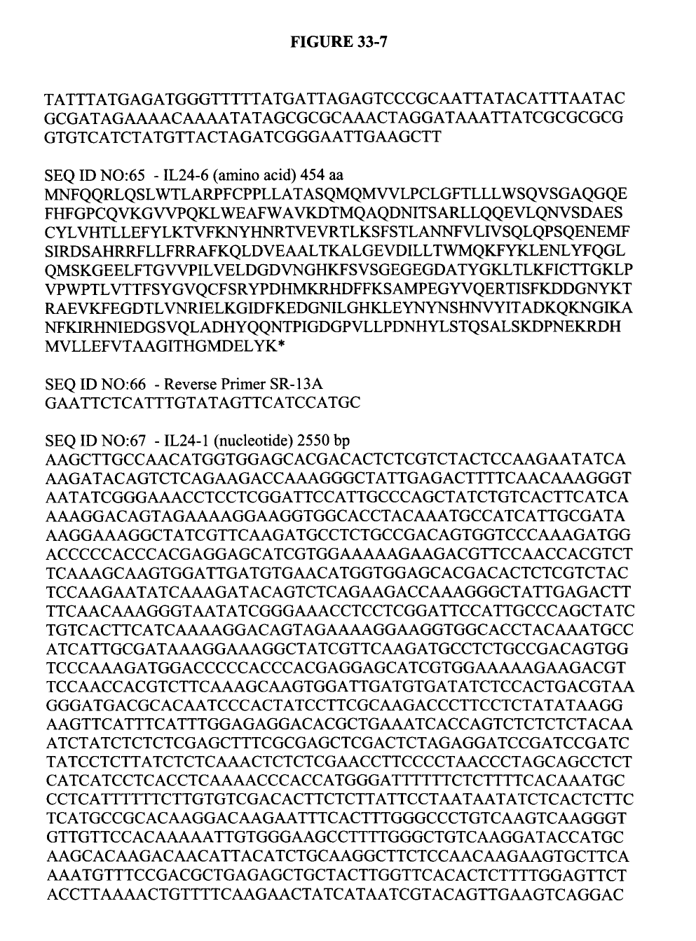

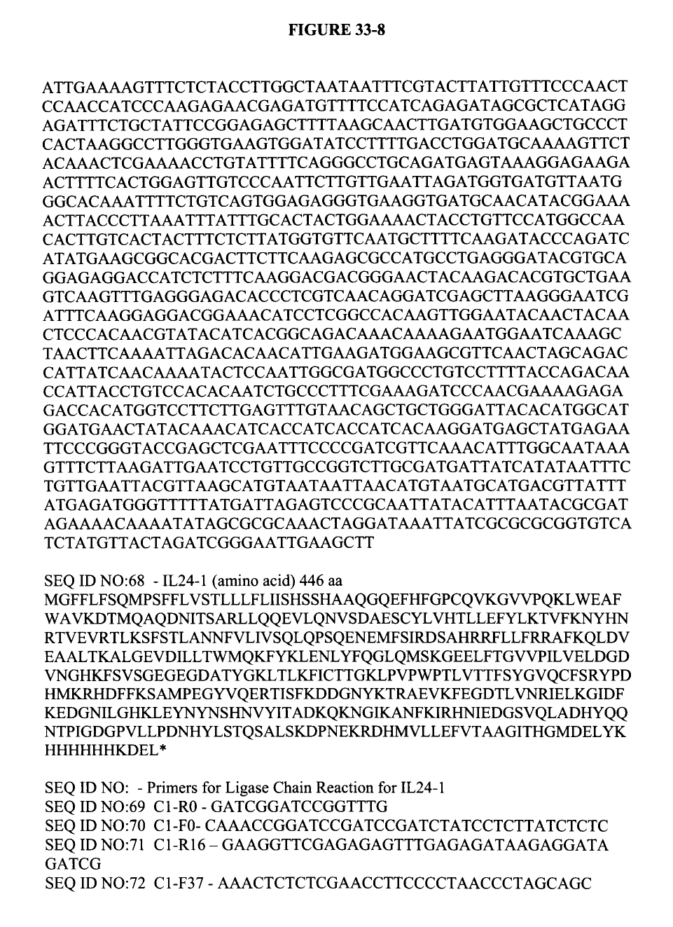

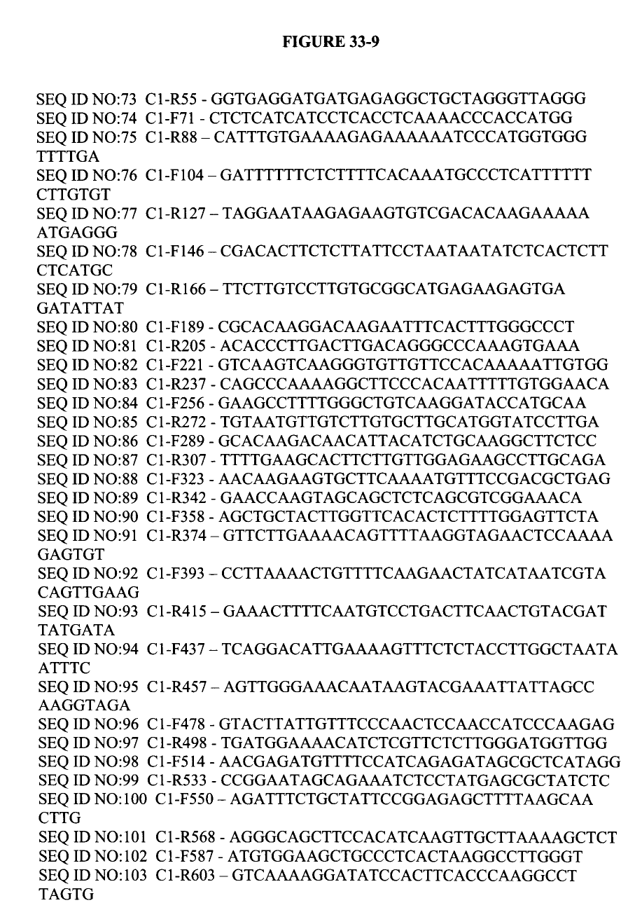

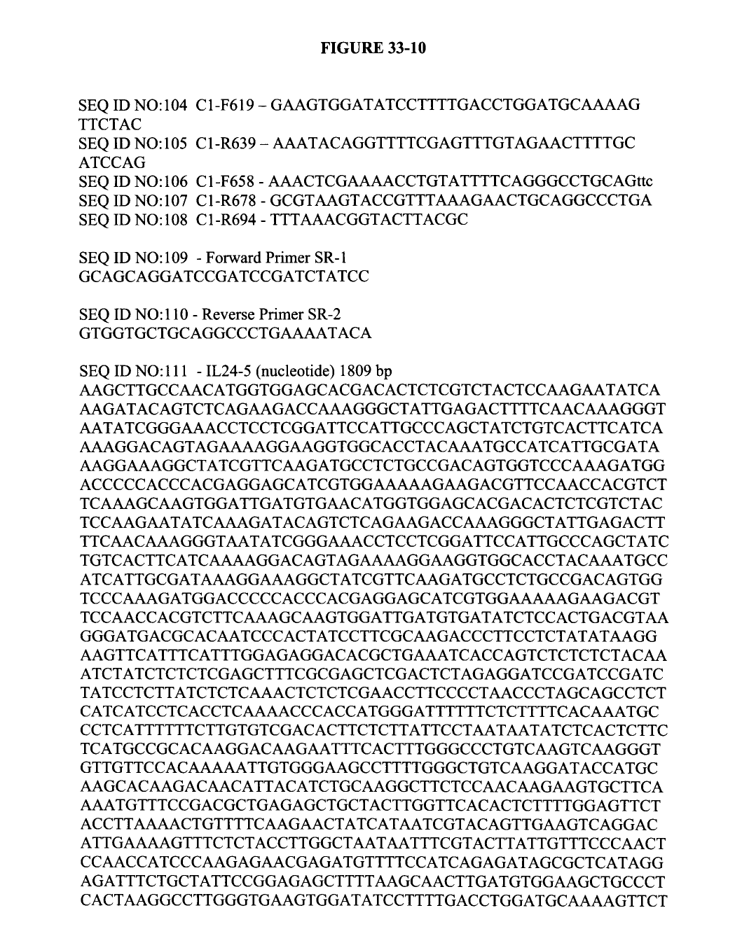

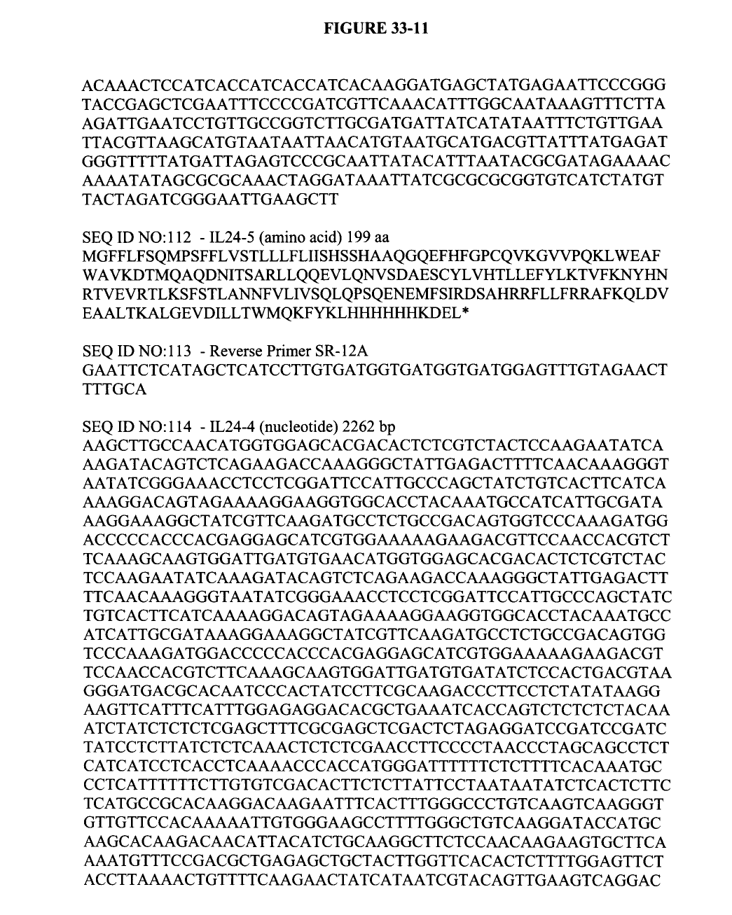

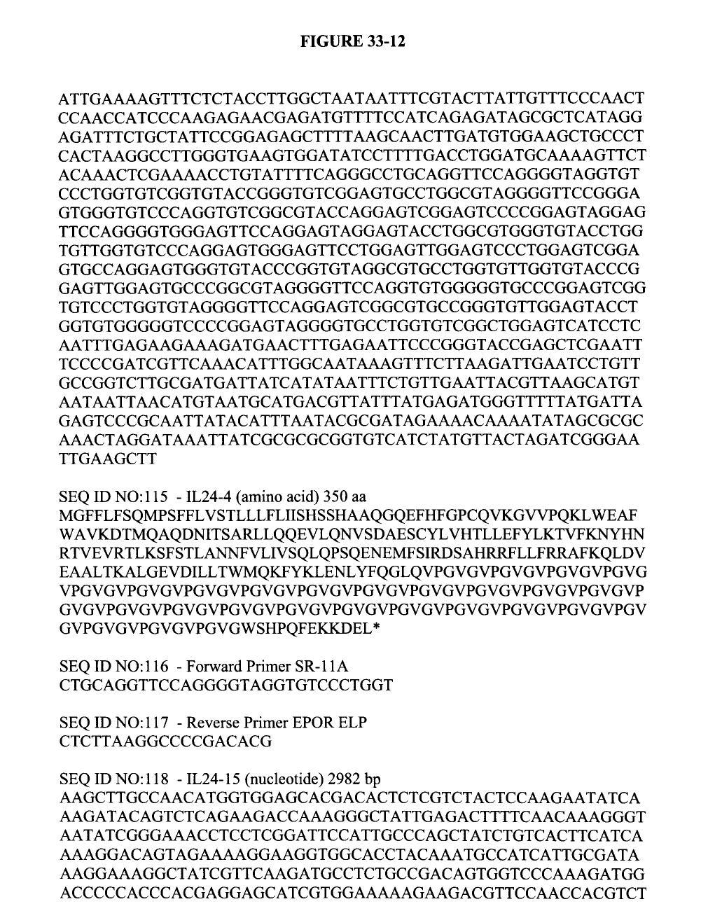

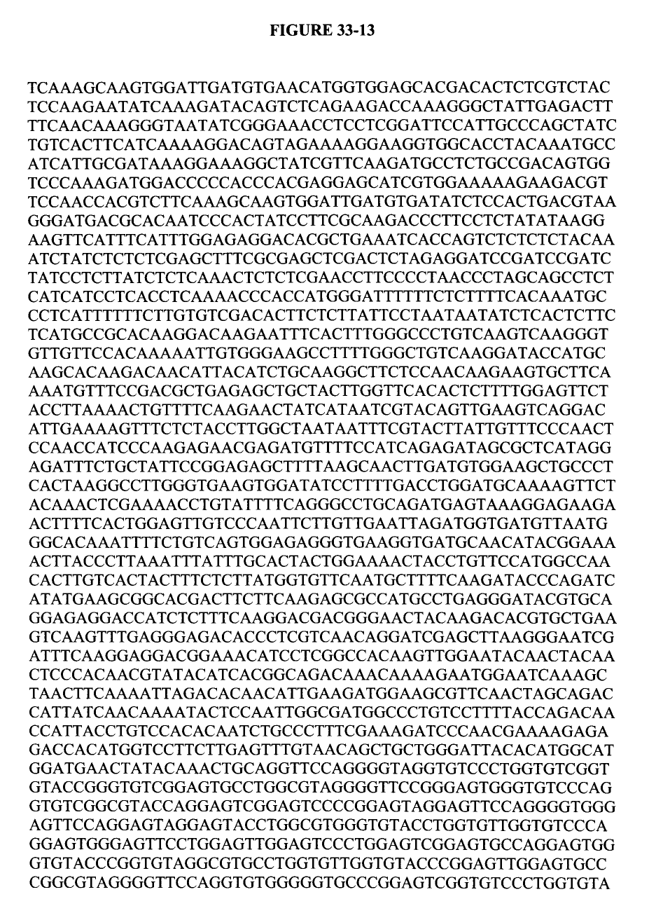

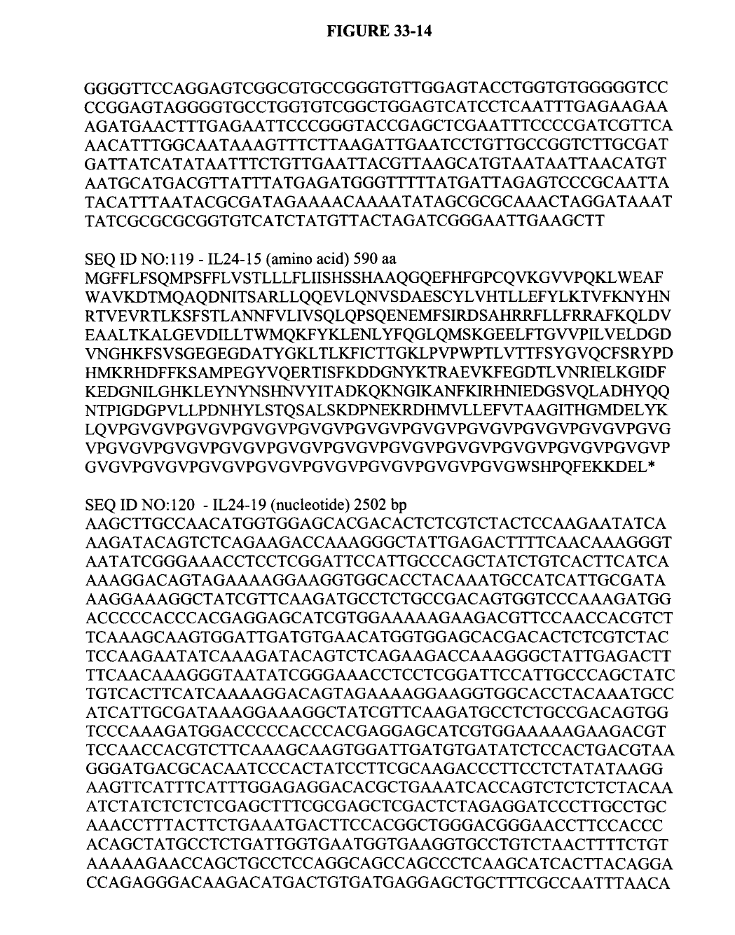

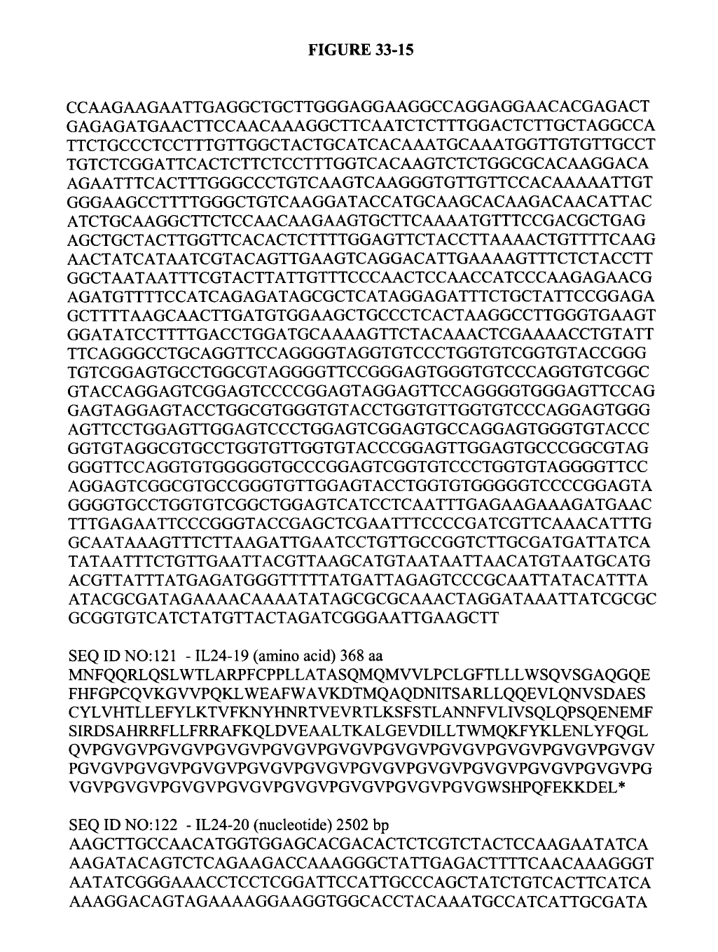

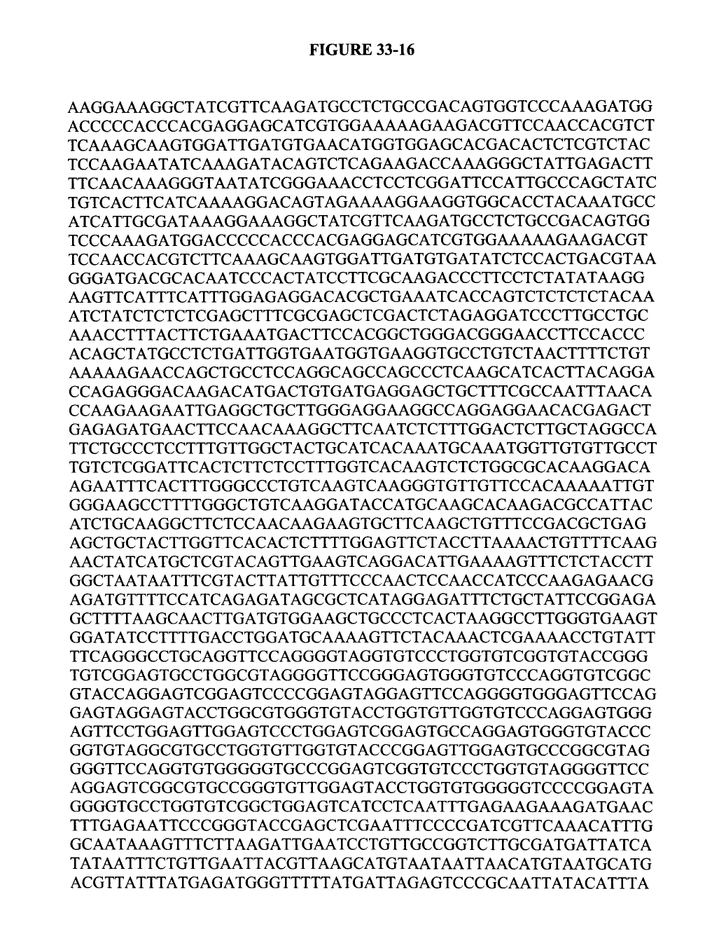

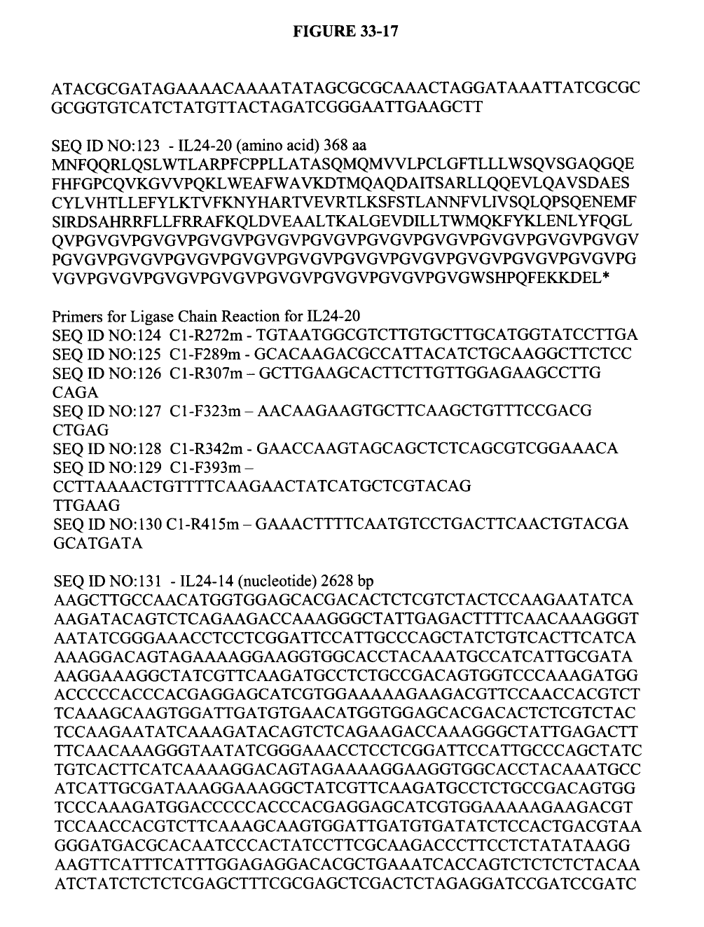

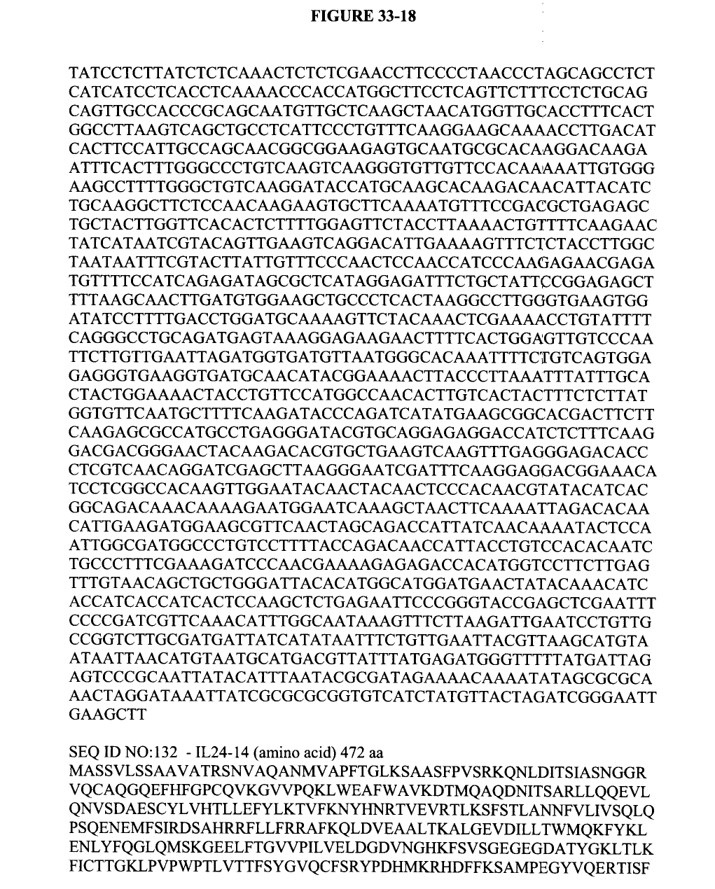

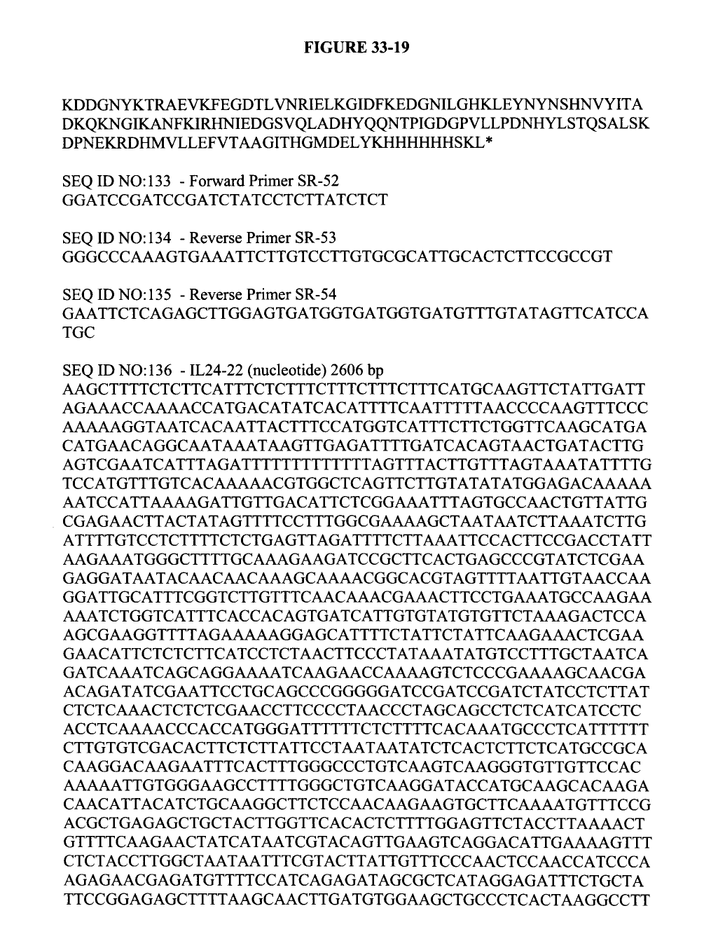

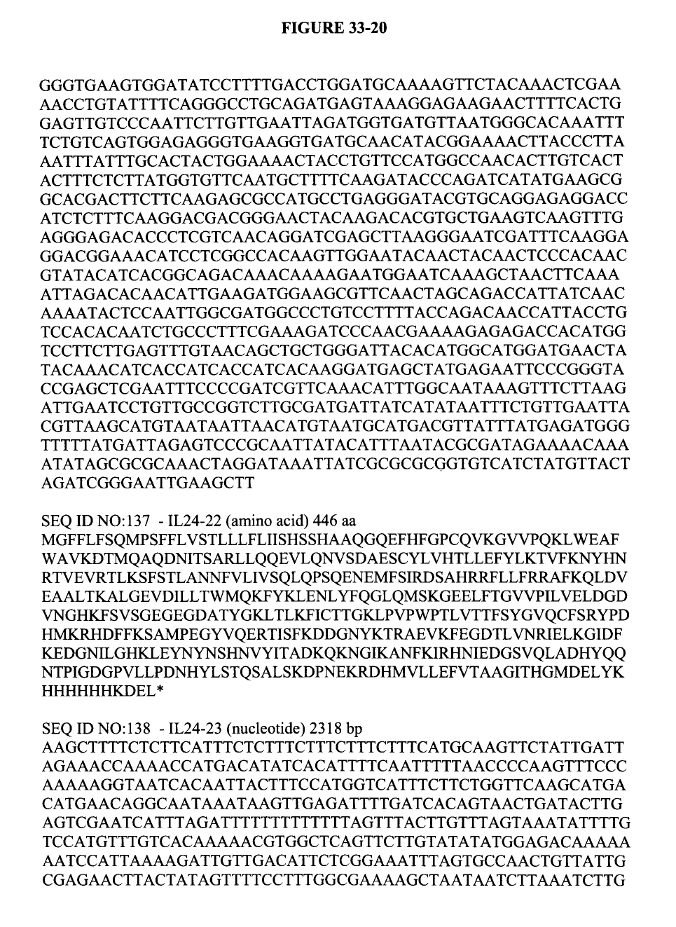

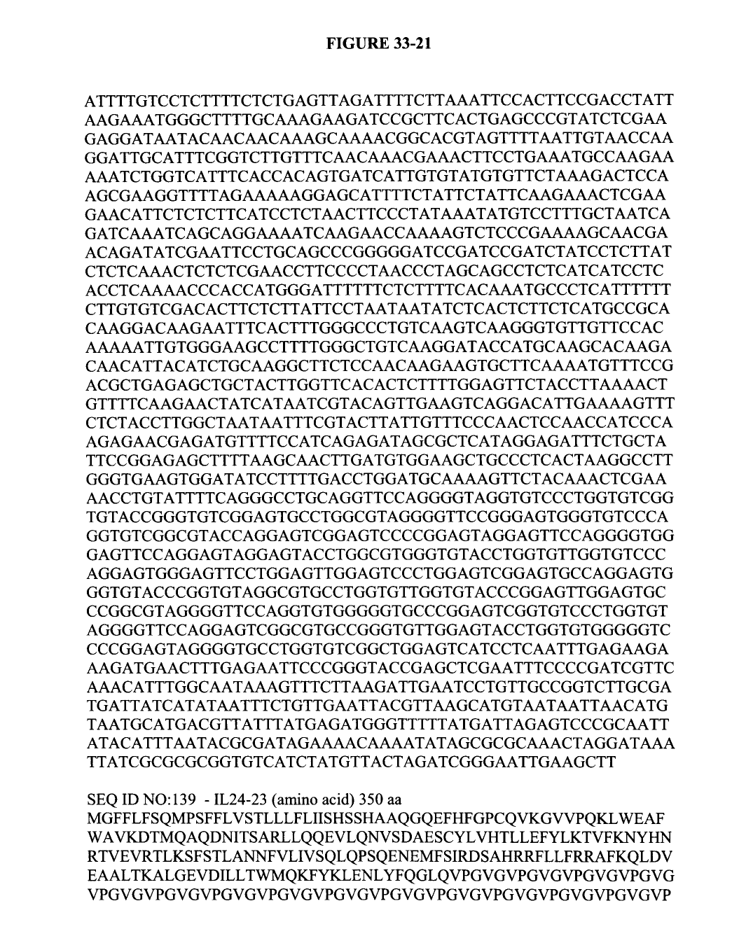

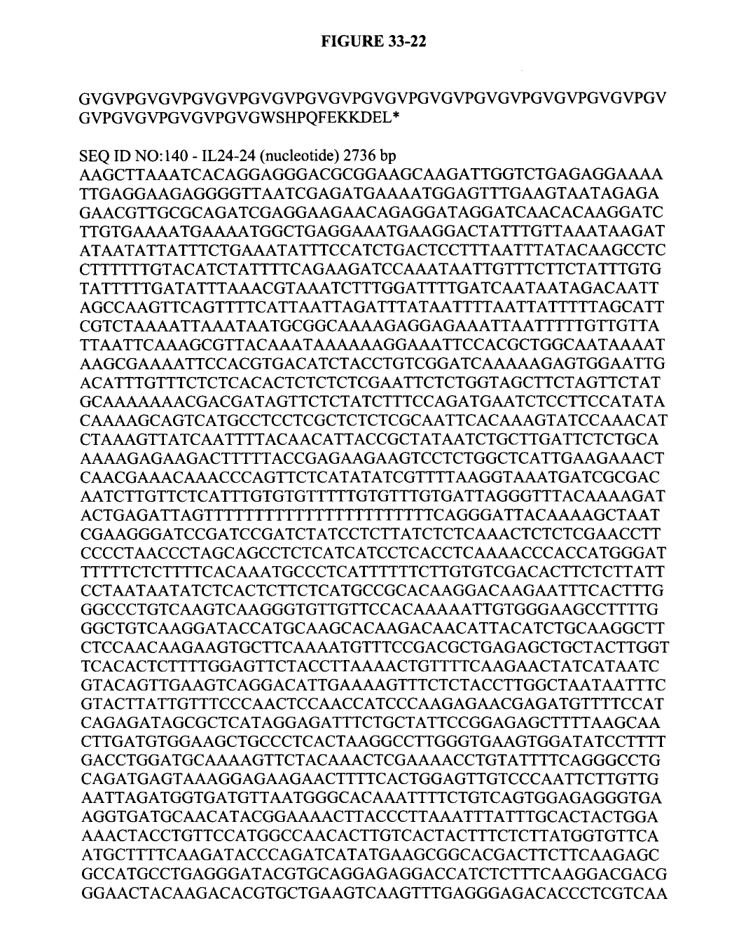

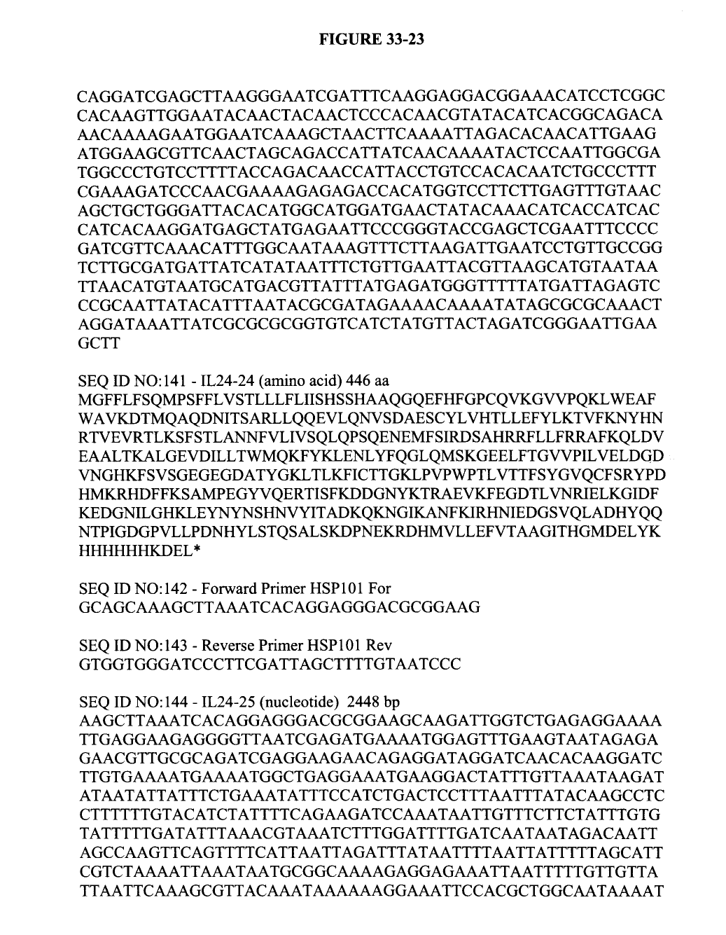

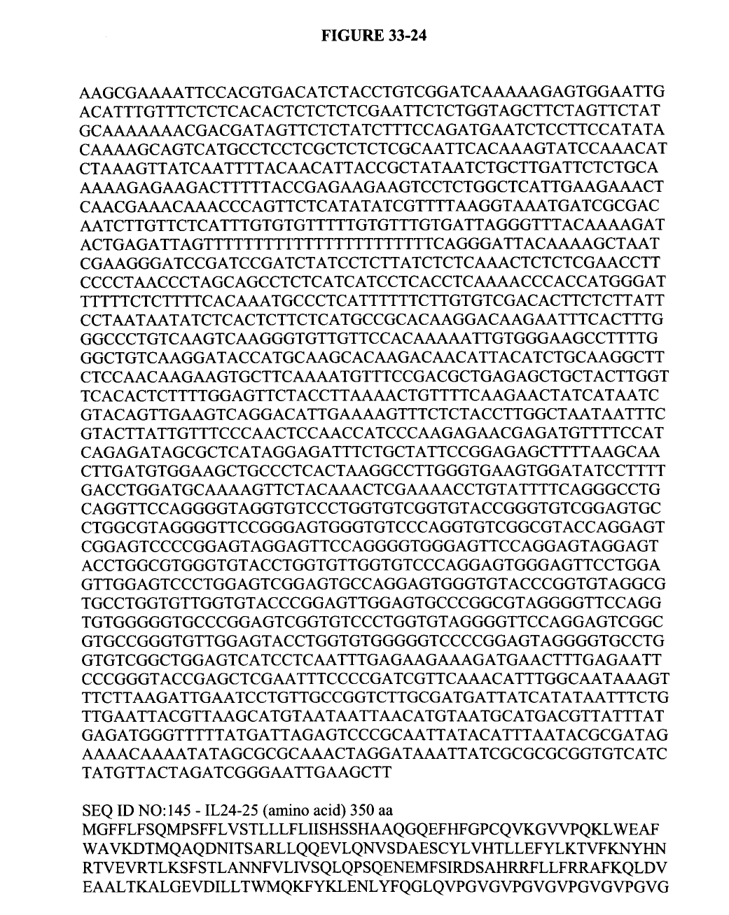

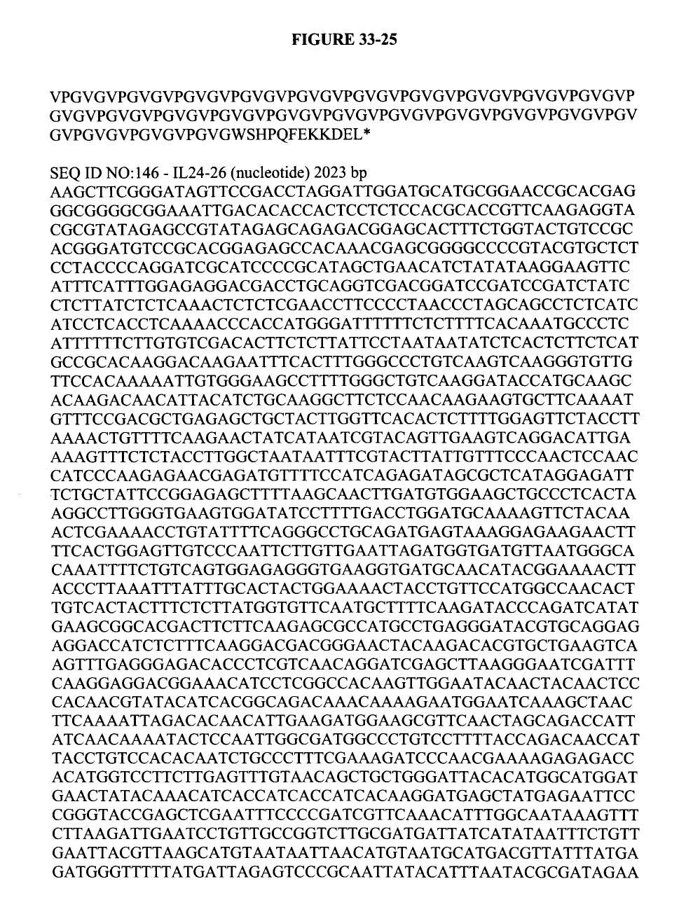

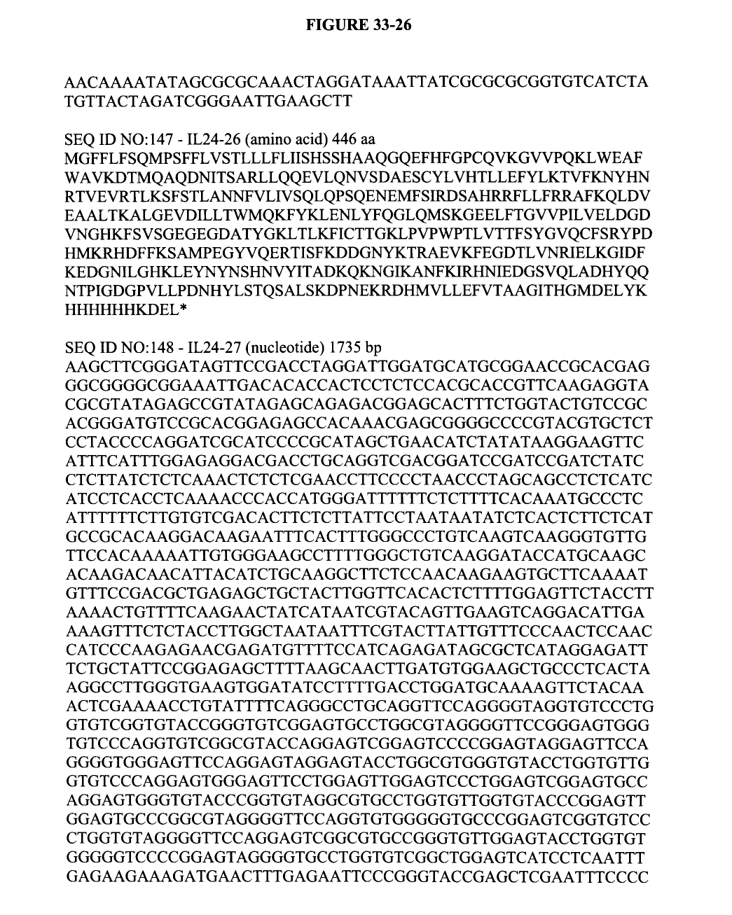

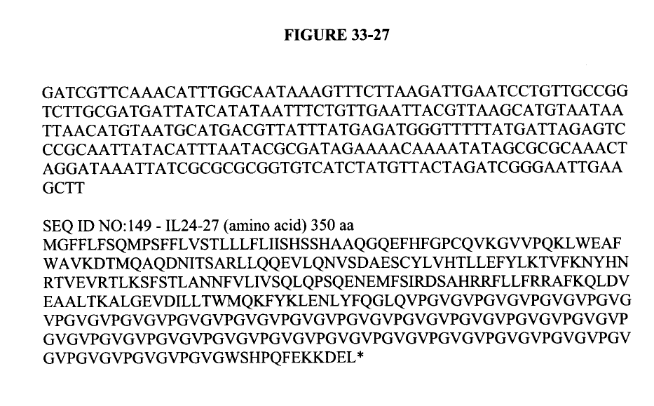

IL-24 can be purified from a plant, a portion of a plant, or cell culture. As an example of the purification protocol, mature tobacco leaves were harvested, extracted and applied to a chelating column, for example a HiTrap chelating column (see Example 10). Fractions were collected and analyzed using western blot analysis (FIG. 32A), and the purified IL-24:GFP protein was incubated with recombinant AcTEV protease (Invitrogen) and analyzed (FIGS. 32B and 32C). The results show that IL-24 is correctly produced in IL-24-1 plants. The GFP-fusion tag can be removed leaving an intact pIL-24 protein (FIG. 32C). As similar purification protocol to obtain IL-24 can be carried out using cell culture. Furthermore, the yield may be optimized by expressing IL-24 under the control of an inducible promoter

The present invention discloses the following sequences:

SEQ ID NOs: 1 and 2 set forth the nucleotide sequence and the deduced amino acid sequence, respectively, of the human interleukin-24 (IL-24) protein.

SEQ ID NOs: 3 and 4 set forth the nucleotide sequence and the deduced amino acid sequence, respectively, of the tobacco codon-optimized human interleukin-24 (IL-24) protein.

SEQ ID NOs: 5 and 6 set forth the nucleotide sequence and the deduced amino acid sequence, respectively, of the IL24-3 clone.

SEQ ID NOs: 7 to 59 set forth the nucleotide sequence of the series of forward and reverse primers that were used in the ligase chain reaction to create a tobacco codon-optimized synthetic clone of IL-24 containing the 5′UTR, signal peptide and mature IL-24 peptide with the stop codon removed and a tobacco etch virus (TEV) protease site.

SEQ ID NO: 60 sets forth the nucleotide sequence of the forward primer SR-3 which is complementary to the 5′UTR region of IL-24 and is designed to add a BamHI site to the start of the 5′UTR region.

SEQ ID NO: 61 sets forth the nucleotide sequence of the reverse primer SR-4 which is complementary to the 3′ region of IL-24 and is designed to add a PstI site after the TEV protease sequence to assist in creating a in-frame translation fusion with smGFP.

SEQ ID NO: 62 sets forth the nucleotide sequence of the forward primer SR-9 which is complementary to the start of smGFP and is designed to add a PstI site in-frame to the smGFP coding sequence.

SEQ ID NO: 63 sets forth the nucleotide sequence of the reverse primer SR-10A which is complementary to the 3′ region of smGFP and is designed to remove a stop codon and add 6 HIS residues, a KDEL retrieval motif, a new stop codon and a EcoRI site to the smGFP coding sequence.

SEQ ID NOs: 64 and 65 set forth the nucleotide sequence and the deduced amino acid sequence, respectively, of the IL24-6 clone.

SEQ ID NO: 66 sets forth the nucleotide sequence of the reverse primer SR-13A which is complementary to the 3′ region of smGFP and is designed to add a stop codon and an EcoRI site to the smGFP coding sequence.

SEQ ID NOs: 67 and 68 set forth the nucleotide sequence and the deduced amino acid sequence, respectively, of the IL24-1 clone.

SEQ ID NOs: 69 to 108 set forth the nucleotide sequence of the series of forward and reverse primers that were used in the ligase chain reaction to create a tobacco codon-optimized synthetic clone of IL-24 containing the tCUP enhancer, Pr1b signal peptide and mature IL-24 peptide with the stop codon removed and a tobacco etch virus (TEV) protease site.

SEQ ID NO: 109 sets forth the nucleotide sequence of the forward primer SR-1 which is complementary to the 5′ region of the tCUP enhancer and is designed to add a BamHI site to the start of the 5′ region.

SEQ ID NO: 110 sets forth the nucleotide sequence of the reverse primer SR-2 which is complementary to the 3′ region of IL-24 and is designed to add a PstI site after the TEV protease sequence to assist in creating a in-frame translation fusion with smGFP.

SEQ ID NOs: 111 and 112 set forth the nucleotide sequence and the deduced amino acid sequence, respectively, of the IL24-5 clone.

SEQ ID NO: 113 sets forth the nucleotide sequence of the reverse primer SR-12A which is complementary to the 3′ region of IL-24 and is designed to remove a stop codon and add 6 HIS residues, a KDEL retrieval motif, a new stop codon and a EcoRI site to the IL-24 coding sequence.

SEQ ID NOs: 114 and 115 set forth the nucleotide sequence and the deduced amino acid sequence, respectively, of the IL24-4 clone.

SEQ ID NO: 116 sets forth the nucleotide sequence of the forward primer SR-11A which is complementary to the start of ELP and is designed to add a PstI site in-frame to the ELP coding sequence.

SEQ ID NO: 117 sets forth the nucleotide sequence of the reverse primer EPOR-ELP which is complementary to the 3′ region of ELP and is designed to add an EcoRI site after the KDEL motif and stop codon.

SEQ ID NOs: 118 and 119 set forth the nucleotide sequence and the deduced amino acid sequence, respectively, of the IL24-15 clone.

SEQ ID NOs: 120 and 121 set forth the nucleotide sequence and the deduced amino acid sequence, respectively, of the IL24-19 clone.

SEQ ID NOs: 122 and 123 set forth the nucleotide sequence and the deduced amino acid sequence, respectively, of the IL24-20 clone.

SEQ ID NOs: 124 to 130 set forth the nucleotide sequence of the series of forward and reverse primers that were used in the ligase chain reaction to create a tobacco codon-optimized synthetic clone of an aglycosylated IL-24 in which the 3 Asn residues are mutated to Ala residues (N85A, N99A and N126A).

SEQ ID NOs: 131 and 132 set forth the nucleotide sequence and the deduced amino acid sequence, respectively, of the IL24-14 clone.

SEQ ID NO: 133 sets forth the nucleotide sequence of the forward primer SR-52 which is complementary to the start of the tCUP enhancer and is designed to add a BamHI site to the start of the enhancer.

SEQ ID NO: 134 sets forth the nucleotide sequence of the reverse primer SR-53 which is complementary to the 5′ region of IL-24 and is designed to add an ApaI site after the ApaI site which occurs in mature IL-24.

SEQ ID NO: 135 sets forth the nucleotide sequence of the reverse primer SR-54 which is complementary to the 3′ region of smGFP and is designed to remove a stop codon and add 6 HIS residues, a SKL peroxisome targeting motif, a new stop codon and a EcoRI site to the smGFP coding sequence.

SEQ ID NO: 136 and 137 set forth the nucleotide sequence and the deduced amino acid sequence, respectively, of the IL24-22 clone.

SEQ ID NO: 138 and 139 set forth the nucleotide sequence and the deduced amino acid sequence, respectively, of the IL24-23 clone.

SEQ ID NO: 140 and 141 set forth the nucleotide sequence and the deduced amino acid sequence, respectively, of the IL24-24 clone.

SEQ ID NO: 142 sets forth the nucleotide sequence of the forward primer HSP101 For which is complementary to the start of the HSP101 promoter and is designed to add a HindIII site to beginning of the promoter sequence.

SEQ ID NO: 143 sets forth the nucleotide sequence of the reverse primer HSP101 Rev which is complementary to the 3′ region of the HSP101 promoter and is designed to add a BamHI site after the end of the promoter sequence.

SEQ ID NO: 144 and 145 set forth the nucleotide sequence and the deduced amino acid sequence, respectively, of the IL24-25 clone.

SEQ ID NO: 146 and 147 set forth the nucleotide sequence and the deduced amino acid sequence, respectively, of the IL24-26 clone.

SEQ ID NO: 148 and 149 set forth the nucleotide sequence and the deduced amino acid sequence, respectively, of the IL24-27 clone.

The present invention will be further illustrated in the following examples.

### Examples

### Example 1

### Construction of IL-24 Clones

The human IL-24 coding sequence (FIG. 1A) (SEQ ID: 1) was analyzed for codon usage and the presence of undesired sequence motifs that could mediate spurious mRNA processing and instability, or methylation of genomic DNA. The plant-optimized IL-24 gene (FIG. 1B) (SEQ ID: 3) was then generated by the ligase chain reaction using synthetic oligonucleotides and a PCR step (Rouillard J M, Lee W, Truan G, Gao X, Zhou X, Gulari E. Gene2Oligo: oligonucleotide design for in vitro gene synthesis. Nucleic Acids Res 2004, 32: W176-80). The resulting PCR product was cloned in the pCR2.1TOPO cloning vector (Invitrogen). Several IL-24 clones were produced as represented in FIG. 2:

IL24-3 (native 5′UTR-native SP::IL-24::TEV::smGFP::His::KDEL)

IL24-3 (SEQ ID NO:5) is a clone designed to express in a constitutive manner which is constructed as per FIGS. 3(A) and 3(B). As seen in FIG. 2, the IL24-3 clone consists of constitutive promoter (CaMV 35S) and terminator (nos) driving the expression of a fusion protein (SEQ ID NO:6) between IL-24 and soluble modified green fluorescent protein (smGFP) with an ER retrieval motif (KDEL). To construct this clone a series of forward and reverse primers (SEQ ID NO:7 to SEQ ID NO:59) were used in a ligase chain reaction (LCR) to create a tobacco codon-optimized synthetic clone of IL-24 containing the 5′UTR, signal peptide and mature IL-24 peptide with the stop codon removed and a tobacco etch virus (TEV) protease site. It was then further amplified by PCR using forward primer SR-3 (SEQ ID NO:60) adding a BamHI site to the 5′UTR region of IL-24. Reverse primer SR-4 (SEQ ID NO:61) added a PstI site after the TEV protease sequence to assist in creating a in-frame translation fusion with smGFP. The PCR fragment was ligated (1) into the EcoRI sites of cloning vector pCR2.1-TOPO (Invitrogen) creating plasmid pTOPO-LCR3 (2) (FIG. 3A). Forward primer SR-9 (SEQ ID NO:62) added a PstI site in-frame to the smGFP coding sequence. Reverse primer SR-10A (SEQ ID NO:63) removed a stop codon and added 6 HIS residues, a KDEL retrieval motif, a new stop codon and a EcoRI site to the smGFP coding sequence. The template for these primers was a pKS+ based vector (Stratagene) pCD3-326 containing the smGFP coding sequence (ABRC Order #8514). The PCR fragment was ligated (3) into the EcoRI sites of cloning vector pCR2.1-TOPO (Invitrogen) creating plasmid pTOPO-smGFP-KDEL (4) (FIG. 3A). As shown in FIG. 3A, plasmid pTOPO-LCR3 contained the 5′UTR, signal peptide, mature IL-24 and TEV protease site and was cut with BamHI and PstI. Plasmid pTOPO-smGFP-KDEL contained smGFP, a HIS-tag, KDEL motif and stop codon and was cut with PstI and EcoRI.

The fragments of pTOPO-LCR3 and pTOPO-smGFP-KDEL were ligated together into the BamHI and EcoRI sites of the cloning vector pBlu2KSP (Stratagene) (1) to create the plasmid pK24-3 (FIG. 3B). Plasmid pK24-3 was cut with BamHI and EcoRI to remove the IL-24-GFP fusion cassette and the fragment was subsequently cloned into the BamHI/EcoRI sites of binary vector pCAMterX (2) (FIG. 3B) to create plasmid pC24-3. Plasmid pC24-3 contained a duplicated 35S enhancer-promoter plus the IL-24 5′UTR and native signal peptide, a NOS (nopaline synthase) polyadenylation sequence and a neomycin phosphotransferase (NPT II) gene conferring host plant kanamycin resistance driven by Nos promoter/terminator for transformation into Agrobacterium.

IL24-6 (Native 5′ UTR-Native SP::IL-24::TEV::smGFP)

IL24-6 (SEQ ID NO:64) is a clone designed to express in a constitutive manner which is constructed as per FIG. 4. As seen in FIG. 2, the IL24-6 clone consists of constitutive promoter (CaMV 35S) and terminator (nos) driving the expression of a fusion protein (SEQ ID NO:65) between IL-24 and smGFP. To construct this clone forward primer SR-3 (SEQ ID NO:60) added a BamHI site to the 5′UTR region of IL-24 (the template was derived from the plasmid pK24-3). Reverse primer SR-13A (SEQ ID NO:66) added a stop codon and an EcoRI site to the smGFP coding sequence. The PCR fragment was ligated into the EcoRI sites of cloning vector pCR2.1-TOPO (Invitrogen) creating plasmid pTOPO-24-6 (1) (FIG. 4). Plasmid pTOPO-24-6 was cut with BamHI and EcoRI to remove the IL-24-GFP fusion cassette and the fragment was subsequently cloned into the BamHI/EcoRI sites of binary vector pCAMterX (2) to create plasmid pC24-6 (FIG. 4). This plasmid contains a duplicated 35S enhancer-promoter plus the IL-24 5′UTR and native signal peptide, a NOS (nopaline synthase) polyadenylation sequence and a neomycin phosphotransferase (NPT II) gene conferring host plant kanamycin resistance driven by Nos promoter/terminator for transformation into Agrobacterium.

IL24-1 (tCUP-Pr1b::IL-24::smGFP::His::KDEL)

IL24-1 (SEQ ID NO:67) is a clone designed to express in a constitutive manner which is constructed as per FIG. 5. As seen in FIG. 2, the IL24-1 clone consists of constitutive promoter (CaMV 35S) and terminator (nos) driving the expression of a fusion protein (SEQ ID NO:68) between IL-24 and soluble modified green fluorescent protein (smGFP) with an ER retrieval motif (KDEL). To construct this clone a series of forward and reverse primers (SEQ ID NO:69 to SEQ ID NO:108) were used in a ligase chain reaction (LCR) to create a tobacco codon-optimized synthetic clone of IL-24 containing the tCUP enhancer, Pr1b signal peptide and mature IL-24 peptide with the stop codon removed and a tobacco etch virus (TEV) protease site. It was then further amplified by PCR using forward primer SR-1 (SEQ ID NO:109) which added a BamHI site to the start of the tCUP enhancer. Reverse primer SR-2 (SEQ ID NO:110) added a PstI site after the TEV protease sequence to assist in creating an in-frame translation fusion with smGFP. The PCR fragment was ligated (1) into the EcoRI sites of cloning vector pCR2.1-TOPO (Invitrogen) creating plasmid pTOPO-LCR1 (FIG. 5). Plasmid pTOPO-LCR1 contains the tCUP enhancer, Pr1b signal peptide, mature IL-24 and TEV protease site and was cut with BamHI and PstI. Plasmid pTOPO-smGFP-KDEL contains smGFP, a HIS-tag, KDEL motif and stop codon and was cut with PstI and EcoRI (see FIG. 3A). The fragments of pTOPO-LCR1 and pTOPO-smGFP-KDEL were ligated together into the BamHI and EcoRI sites of the cloning vector pBlu2KSP (Stratagene) (2) to create the plasmid pK24-1 (FIG. 5). Plasmid pK24-1 was cut with BamHI and EcoRI to remove the IL-24-GFP fusion cassette and the fragment was subsequently cloned into the BamHI/EcoRI sites of binary vector pCAMterX (3) (FIG. 5) to create plasmid pC24-1. This plasmid contains a duplicated 35S enhancer-promoter plus tCUP leader sequence, a NOS (nopaline synthase) polyadenylation sequence and a neomycin phosphotransferase (NPT II) gene conferring host plant kanamycin resistance driven by Nos promoter/terminator for transformation into Agrobacterium.

IL24-5 (tCUP-Pr1b::IL-24::His::KDEL)

IL24-5 (SEQ ID NO:111) is a clone designed to express in a constitutive manner which is constructed as per FIG. 6. As seen in FIG. 2, the IL24-5 clone consists of constitutive promoter (CaMV 35S) and terminator (nos) driving the expression of a IL-24 protein (SEQ ID NO:112) with an ER-retrieval motif (KDEL). To construct this clone forward primer SR-1 (SEQ ID NO:109) added a BamHI site to the tCUP enhancer (the template was derived from the plasmid pK24-1). Reverse primer SR-12A (SEQ ID NO:113) removed a stop codon and added 6 HIS residues, a KDEL retrieval motif, a new stop codon and a EcoRI site to the IL-24 coding sequence. The PCR fragment was ligated into the EcoRI sites of cloning vector pCR2.1-TOPO (Invitrogen) creating plasmid pTOPO-24-5 (1) (FIG. 6). Plasmid pTOPO-24-5 was cut with BamHI and EcoRI to remove the IL-24-KDEL fragment and was subsequently cloned into the BamHI/EcoRI sites of binary vector pCAMterX (2) (FIG. 6) to create plasmid pC24-5. This plasmid contains a duplicated 35S enhancer-promoter plus tCUP leader sequence, a NOS (nopaline synthase) polyadenylation sequence and a neomycin phosphotransferase (NPT II) gene conferring host plant kanamycin resistance driven by Nos promoter/terminator for transformation into Agrobacterium.

IL24-4 (tCUP-Pr1b::IL-24::TEV::ELP::Streplk:KDEL)

IL24-4 (SEQ ID NO:114) is a clone designed to express in a constitutive manner which is constructed as per FIG. 7. As seen in FIG. 2, the IL24-4 clone consists of constitutive promoter (CaMV 35S) and terminator (nos) driving the expression of a fusion protein (SEQ ID NO:115) between IL-24 and an elastin-like polypeptide (ELP) with an ER retrieval motif (KDEL). To construct this clone forward primer SR-11A (SEQ ID NO:116) added a PstI site to the start of the ELP coding sequence (the template was derived from the plasmid EPOE). Reverse primer EPOR ELP (SEQ ID NO:117) added an EcoRI site after the KDEL motif and stop codon. The PCR fragment was ligated into the EcoRI sites of cloning vector pCR2.1-TOPO (Invitrogen) creating plasmid pTOPO-ELP-KDEL (1) (FIG. 7). Plasmid pTOPO-LCR1 contains the tCUP enhancer, Pr1b signal peptide, mature IL-24 and TEV protease site and was cut with BamHI and PstI. Plasmid pTOPO-ELP-KDEL contains ELP, a StrepII-tag, KDEL motif and stop codon and was cut with PstI and EcoRI (see FIG. 7). The fragments of pTOPO-LCR1 and pTOPO-ELP-KDEL were ligated together into the BamHI and EcoRI sites of the cloning vector pBlu2KSP (Stratagene) (2) to create the plasmid pK24-4 (FIG. 7). Plasmid pK24-4 was cut with BamHI and EcoRI to remove the IL-24-ELP fusion cassette and the fragment was subsequently cloned into the BamHI/EcoRI sites of binary vector pCAMterX (3) (FIG. 7) to create plasmid pC24-4. This plasmid contains a duplicated 35S enhancer-promoter plus tCUP leader sequence, a NOS (nopaline synthase) polyadenylation sequence\and a neomycin phosphotransferase (NPT II) gene conferring host plant kanamycin resistance driven by Nos promoter/terminator for transformation into Agrobacterium.

IL24-15 (tCUP-Pr1b::IL-24::TEV::smGFP::ELP::StrepII::KDEL)

IL24-15 (SEQ ID NO:118) is a clone designed to express in a constitutive manner which is constructed as per FIG. 8. As seen in FIG. 2, the IL24-15 clone consists of constitutive promoter (CaMV 35S) and terminator (nos) driving the expression of a fusion protein (SEQ ID NO:119) between IL-24, smGFP and an elastin-like polypeptide (ELP) with an ER retrieval motif (KDEL). Plasmid pK24-1 contains the tCUP enhancer, Pr1b signal peptide, mature IL-24, TEV protease site, smGFP, HIS-tag and KDEL motif and was cut with PstI to remove smGFP. Plasmid pK24-4 contains the tCUP enhancer, Pr1b signal peptide, mature IL-24, TEV protease site, ELP, StrepII-tag and KDEL motif and was cut with PstI and treated with calf intestinal phosphatase (see FIG. 8). The fragments of pK24-1 and pK24-4 were ligated together into the PstI sites of plasmid pK24-4 (1) to create the plasmid pK24-15 (FIG. 8). Plasmid pK24-15 was cut with BamHI and EcoRI to remove the IL-24-smGFP-ELP fusion cassette and the fragment was subsequently cloned into the BamHI/EcoRI sites of binary vector pCAMterX (2) (FIG. 8) to create plasmid pC24-15. This plasmid contains a duplicated 35S enhancer-promoter plus tCUP leader sequence, a NOS (nopaline synthase) polyadenylation sequence and a neomycin phosphotransferase (NPT II) gene conferring host plant kanamycin resistance driven by Nos promoter/terminator for transformation into Agrobacterium.

IL24-19 (native 5′UTR-native SP::IL-24::TEV::ELP::StrepII::KDEL)

IL24-19 (SEQ ID NO:120) is a clone designed to express in a constitutive manner which is constructed as per FIG. 9. As seen in FIG. 2, the IL24-19 clone consists of constitutive promoter (CaMV 35S) and terminator (nos) driving the expression of a fusion protein (SEQ ID NO:121) between IL-24 and an elastin-like polypeptide (ELP) with an ER retrieval motif (KDEL). Plasmid pK24-3 contains the 5′UTR, signal peptide, mature IL-24, TEV protease site, smGFP, HIS-tag and KDEL motif and was cut with BamHI and ApaI. Plasmid pK24-4 contains the tCUP enhancer, Pr1b signal peptide, mature IL-24, TEV protease site, ELP, StrepII-tag and KDEL motif and was cut with ApaI and EcoRI (see FIG. 9). The fragments of pK24-3 and pK24-4 were ligated together into the BamHI and EcoRI sites of the cloning vector pBlu2KSP (Stratagene) (1) to create the plasmid pK24-19 (FIG. 9). Plasmid pK24-19 was cut with BamHI and EcoRI to remove the IL-24-ELP fusion cassette and the fragment was subsequently cloned into the BamHI/EcoRI sites of binary vector pCAMterX (2) (FIG. 9) to create plasmid pC24-19. This plasmid contains a duplicated 35S enhancer-promoter plus the IL-24 5′UTR and native signal peptide, a NOS (nopaline synthase) polyadenylation sequence and a neomycin phosphotransferase (NPT II) gene conferring host plant kanamycin resistance driven by Nos promoter/terminator for transformation into Agrobacterium.

IL24-20 (Native S′UTR-native SP::aglycosylated IL-24::ELP::StrepII::KDEL)

IL24-20 (SEQ ID NO:122) is a clone designed to express in a constitutive manner which is constructed as per FIG. 10. As seen in FIG. 2, the IL24-20 clone consists of constitutive promoter (CaMV 35S) and terminator (nos) driving the expression of a fusion protein (SEQ ID NO:123) between an aglycosylated IL-24 and an elastin-like polypeptide (ELP) with an ER retrieval motif (KDEL). To construct this clone a series of forward and reverse primers (SEQ ID NO:124 to SEQ ID NO:130) were used in a ligase chain reaction (LCR) to create a tobacco codon-optimized synthetic clone of an aglycosylated IL-24 in which the 3 Asn residues are mutated to Ala residues (N85A, N99A and N126A). It also contains the tCUP enhancer, Pr1b signal peptide and mature IL-24 peptide with the stop codon removed and a tobacco etch virus (TEV) protease site. It was then further amplified by PCR using forward primer SR-1 (SEQ ID NO:109) adding a BamHI site to the start of the tCUP enhancer. Reverse primer SR-2 (SEQ ID NO:110) added a PstI site after the TEV protease sequence to assist in creating a in-frame translation fusion with ELP. The PCR fragment was ligated (1) into the EcoRI sites of cloning vector pCR2.1-TOPO (Invitrogen) creating plasmid pTOPO-LCR20 (FIG. 10). Plasmid pTOPO-LCR20 contains the tCUP enhancer, Pr1b signal peptide, mature IL-24 and TEV protease site and was cut with ApaI and PstI. Plasmid pK24-19 contains the 5′UTR, signal peptide, mature IL-24, TEV protease site, ELP, StrepII-tag and KDEL motif and was cut with BamHI, PstI and EcoRI (see FIG. 10). The fragments of pTOPO-LCR20 and BamHI/PstI fragment of pK24-19 were ligated together into the BamHI and PstI sites of the cloning vector pBlu2KSP (Stratagene). This intermediate plasmid was cut with PstI and EcoRI, and the PstI/EcoRI fragment of plasmid pK24-19 was ligated into the PstI and EcoRI sites, creating plasmid pK24-20 (2) (FIG. 10). Plasmid pK24-20 was cut with BamHI and EcoRI to remove the IL-24-ELP fusion cassette and the fragment was subsequently cloned into the BamHI/EcoRI sites of binary vector pCAMterX (3) (FIG. 10) to create plasmid pC24-20. This plasmid contains a duplicated 35S enhancer-promoter plus the IL-24 5′UTR and native signal peptide, a NOS (nopaline synthase) polyadenylation sequence and a neomycin phosphotransferase (NPT II) gene conferring host plant kanamycin resistance driven by Nos promoter/terminator for transformation into Agrobacterium.

IL24-14 (tCUP-RBCS TP::IL-24::TEV::smGFP::His::SKL)

IL24-14 (SEQ ID NO:131) is a clone designed to express in a constitutive manner which is constructed as per FIGS. 11A and B. As seen in FIG. 2, the IL24-14 clone consists of constitutive promoter (CaMV 35S) and terminator (nos) driving the expression of a fusion protein (SEQ ID NO:132) between IL-24 and smGFP targeted to the chloroplasts using a transit peptide and to the peroxisomes using a SKL motif. To construct this clone forward primer SR-52 (SEQ ID NO:133) added a BamHI site to the start of the tCUP enhancer and Rubisco transit peptide (the template was derived from the plasmid E-RG). Reverse primer SR-53 (SEQ ID NO:134) added an ApaI site after the ApaI site which occurs in mature IL-24. The PCR fragment was ligated into the EcoRI sites of cloning vector pCR2.1-TOPO (Invitrogen) creating plasmid pTOPO-RubiscoTP (1) (FIG. 11A). Forward primer SR-9 (SEQ ID NO:62) adds a PstI site in-frame to the smGFP coding sequence. Reverse primer SR-54 (SEQ ID NO:135) removed a stop codon and added 6 HIS residues, a SKL peroxisome targeting motif, a new stop codon and a EcoRI site to the smGFP coding sequence. The template for these primers was a pKS+ based vector (Stratagene) pCD3-326 containing the smGFP coding sequence (ABRC Order #8514). The PCR fragment was ligated into the EcoRI sites of cloning vector pCR2.1-TOPO (Invitrogen) creating plasmid pTOPO-smGFP-SKL (2) (FIG. 11A). Plasmid pTOPO-RubiscoTP contains the tCUP enhancer, Rubisco transit peptide, and a fragment of mature IL-24 until the ApaI site, and was cut with BamHI and ApaI. Plasmid pK24-1 contains the tCUP enhancer, Pr1b signal peptide, mature IL-24, TEV protease site, smGFP, HIS-tag and a KDEL motif, was cut with ApaI and PstI. These fragments were ligated together into the BamHI and PstI sites of the cloning vector pBlu2KSP (Stratagene) to create an intermediate plasmid, and was subsequently cut with PstI and EcoRI. Plasmid pTOPO-smGFP-SKL contains smGFP, a HIS-tag, SKL motif and stop codon and was cut with PstI and EcoRI. The fragment of pTOPO-smGFP-KDEL was ligated into the PstI and EcoRI sites of the intermediate plasmid (1) to create the plasmid pK24-14 (FIG. 11B). Plasmid pK24-14 was cut with BamHI and EcoRI to remove the IL-24-GFP fusion cassette and the fragment was subsequently cloned into the BamHI/EcoRI sites of binary vector pCAMterX (2) (FIG. 11B) to create plasmid pC24-14. This plasmid contains a duplicated 35S enhancer-promoter plus tCUP leader sequence, a NOS (nopaline synthase) polyadenylation sequence and a neomycin phosphotransferase (NPT II) gene conferring host plant kanamycin resistance driven by Nos promoter/terminator for transformation into Agrobacterium.

IL24-22 (HSP18.2 Promoter-tCUP-Pr1b-IL-24::smGFP::His::KDEL)

IL24-22 (SEQ ID NO:136) is a clone designed to express in a heat shock inducible manner which is constructed as per FIG. 12. As seen in FIG. 2, the IL24-22 clone consists of inducible promoter (HSP 18.2) and terminator (nos) driving the expression of a fusion protein (SEQ ID NO:137) between IL-24 and smGFP with an ER retrieval motif (KDEL). Plasmid pTT101 contains the HSP18.2 promoter with a multiple cloning site (MCS) and nos terminator and was cut with BamHI and HindIII. Plasmid pC24-1 contains the CaMV 35S′ promoter, tCUP enhancer, Prib signal peptide, mature IL-24, TEV protease site, smGFP, HIS-tag and KDEL motif and was cut with BamHI and HindIII. The fragments of pTT101 and pC24-1 were ligated together into the HindIII site of the cloning vector pBlu2KSP (Stratagene) (1) treated with calf intestinal phosphatase to create the plasmid pK24-22 (FIG. 12). Plasmid pK24-22 was cut with HindIII to remove the HSP18.2 promoter-IL-24-GFP-nos terminator cassette and the fragment was subsequently cloned into the HindIII site of binary vector pRD400 (2) (FIG. 12) treated with calf intestinal phosphatase to create plasmid pRD24-22. This plasmid contains a neomycin phosphotransferase (NPT II) gene conferring host plant kanamycin resistance driven by Nos promoter/terminator for transformation into Agrobacterium.

IL24-23 (HSP18.2 Promoter-tCUP-Pr1b-IL-24::ELP::His::KDEL)

IL24-23 (SEQ ID NO:138) is a clone designed to express in a heat shock inducible manner which is constructed as per FIG. 13. As seen in FIG. 2, the IL24-23 clone consists of inducible promoter (HSP 18.2) and terminator (nos) driving the expression of a fusion protein (SEQ ID NO:139) between IL-24 and ELP with an ER retrieval motif (KDEL). Plasmid pTT101 contains the HSP18.2 promoter with a multiple cloning site (MCS) and nos terminator and was cut with BamHI and HindIII. Plasmid pC24-4 contains the CaMV 35S′ promoter, tCUP enhancer, Pr1b signal peptide, mature IL-24, TEV protease site, ELP, HIS-tag and KDEL motif and was cut with BamHI and HindIII. The fragments of pTT101 and pC24-4 were ligated together into the HindIII site of the cloning vector pBlu2KSP (Stratagene) (1) treated with calf intestinal phosphatase to create the plasmid pK24-23 (FIG. 13). Plasmid pK24-23 was cut with HindIII to remove the HSP18.2 promoter-IL-24-ELP-nos terminator cassette and the fragment was subsequently cloned into the HindIII site of binary vector pRD400 (2) (FIG. 13) treated with calf intestinal phosphatase to create plasmid pRD24-23. This plasmid contains a neomycin phosphotransferase (NPT II) gene conferring host plant kanamycin resistance driven by Nos promoter/terminator for transformation into Agrobacterium.

IL24-24 (HSP101 Promoter-tCUP-Pr1b-IL-24::smGFP::His::KDEL)

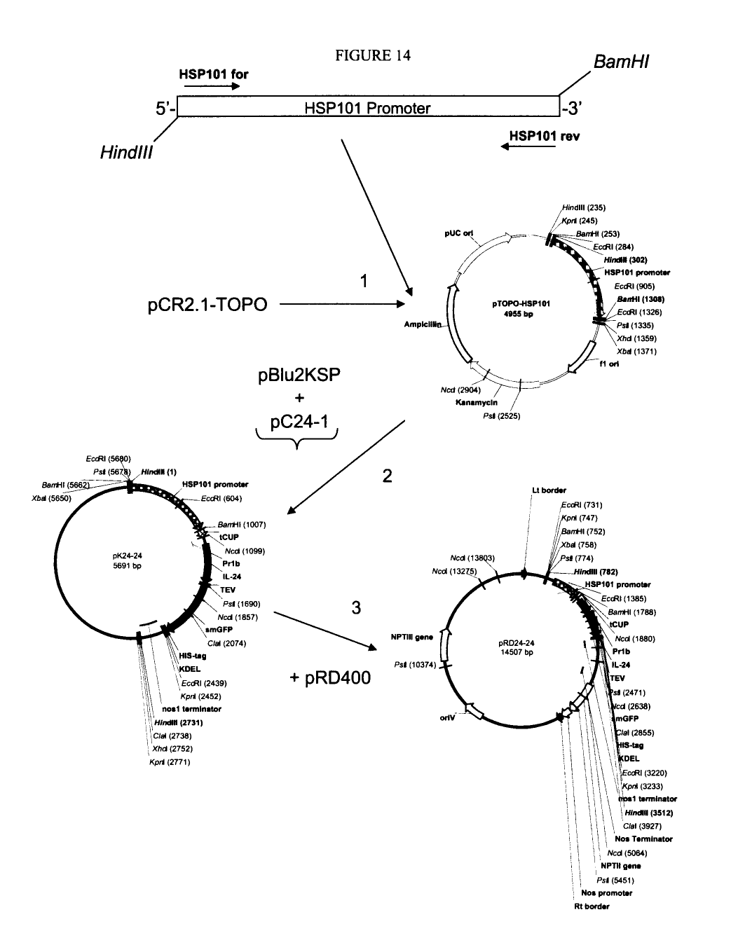

IL24-24 (SEQ ID NO:140) is a clone designed to express in a heat shock inducible manner which is constructed as per FIG. 14. As seen in FIG. 14, the IL24-24 clone consists of inducible promoter (HSP 101) and terminator (nos) driving the expression of a fusion protein (SEQ ID NO:140) between IL-24 and smGFP with an ER retrieval motif (KDEL). The HSP 101 promoter was amplified by PCR from Arabidopsis genomic DNA by primers HSP101 For (SEQ ID NO:142 (5′-GCAGCAAAGCTTAAATCACAGGAGGGACGCGGAAG-3′) adding a HindIII site and HSP101 Rev (SEQ ID NO:143 (5′-GTGGTGGGATCCCTTCGATTAGCTTTTGTAATCCC-3′) adding a BamHI site at their respective ends. The PCR fragment was ligated into the EcoRI sites of cloning vector pCR2.1-TOPO (Invitrogen) creating plasmid pTOPO-HSP101 (1) (FIG. 14). Plasmid pTOPO-HSP101 was cut with HindIII and BamHI to remove the HSP 101 promoter fragment. Plasmid pC24-1 contains the CaMV 35S′ promoter, tCUP enhancer, Pr1b signal peptide, mature IL-24, TEV protease site, smGFP, HIS-tag and KDEL motif and was cut with BamHI and HindIII (see FIG. 14). The HSP101 fragment was ligated together with the pC24-1 fragment into the HindIII site of the cloning vector pBlu2KSP (Stratagene) (1) treated with calf intestinal phosphatase to create the plasmid pK24-24 (FIG. 14). Plasmid pK24-24 was cut with HindIII to remove the HSP101 promoter-IL-24-GFP-nos terminator cassette and the fragment was subsequently cloned into the HindIII site of binary vector pRD400 (3) (FIG. 14) treated with calf intestinal phosphatase to create plasmid pRD24-24. Note that this plasmid contains a NPT II gene conferring host plant kanamycin resistance driven by Nos promoter/terminator (Ref) for transformation into Agrobacterium.

IL24-25 (HSP101 Promoter-tCUP-Pr1b-IL-24::ELP::His::KDEL)

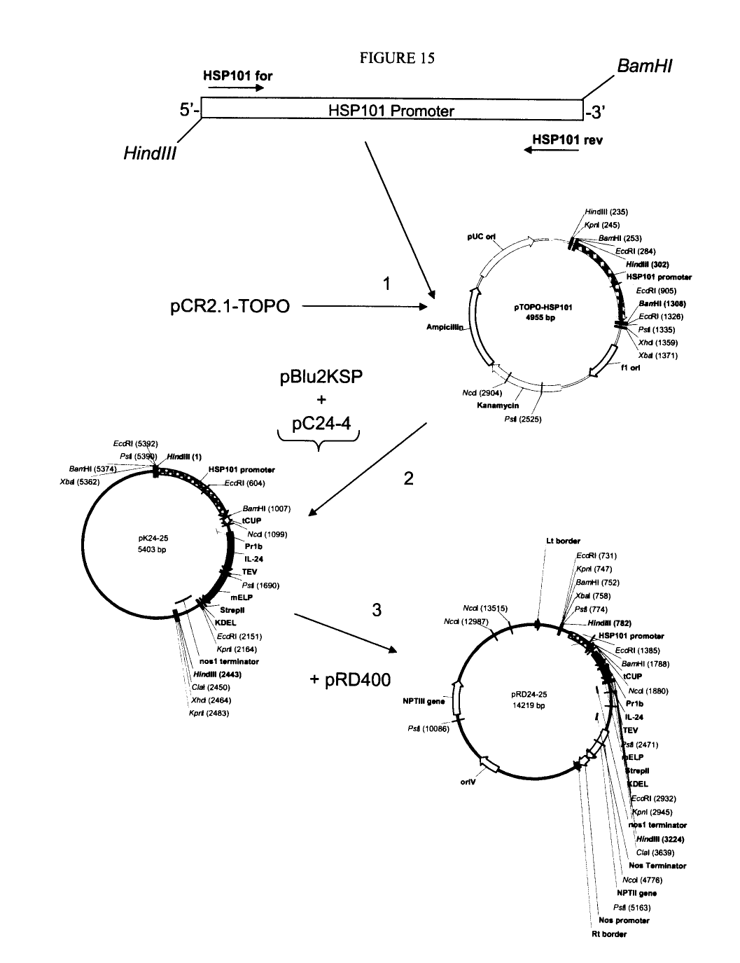

IL24-25 (SEQ ID NO:144) is a clone designed to express in a heat shock inducible manner which is constructed as per FIG. 15. As seen in FIG. 15, the IL24-25 clone consists of inducible promoter (HSP 101) and terminator (nos) driving the expression of a fusion protein (SEQ ID NO:144) between IL-24 and ELP with an ER retrieval motif (KDEL). The HSP 101 promoter was amplified by PCR from Arabidopsis genomic DNA by primers HSP101 For (SEQ ID NO:142 (5′-GCAGCAAAGCTTAAATCACAGGAGGGACGCGGAAG-3′) adding a HindIII site and HSP101 Rev (SEQ ID NO:143 (5′-GTGGTGGGATCCCTTCGATTAGCTTTTGTAATCCC-3′) adding a BamHI site at their respective ends. The PCR fragment was ligated into the EcoRI sites of cloning vector pCR2.1-TOPO (Invitrogen) creating plasmid pTOPO-HSP101 (1) (FIG. 15). Plasmid pTOPO-HSP101 was cut with HindIII and BamHI to remove the HSP 101 promoter fragment. Plasmid pC24-4 contains the CaMV 35S′ promoter, tCUP enhancer, Pr1b signal peptide, mature IL-24, TEV protease site, ELP, HIS-tag and KDEL motif and was cut with BamHI and HindIII (see FIG. 15). The HSP101 fragment was ligated together with the pC24-4 fragment into the HindIII site of the cloning vector pBlu2KSP (Stratagene) (1) treated with calf intestinal phosphatase to create the plasmid pK24-25 (FIG. 15). Plasmid pK24-25 was cut with HindIII to remove the HSP101 promoter-IL-24-ELP-nos terminator cassette and the fragment was subsequently cloned into the HindIII site of binary vector pRD400 (3) (FIG. 15) treated with calf intestinal phosphatase to create plasmid pRD24-25. Note that this plasmid contains a NPT II gene conferring host plant kanamycin resistance driven by Nos promoter/terminator (Ref) for transformation into Agrobacterium.

IL24-26 (AlcA Promoter-tCUP-Pr1b-IL-24::smGFP::His::KDEL)

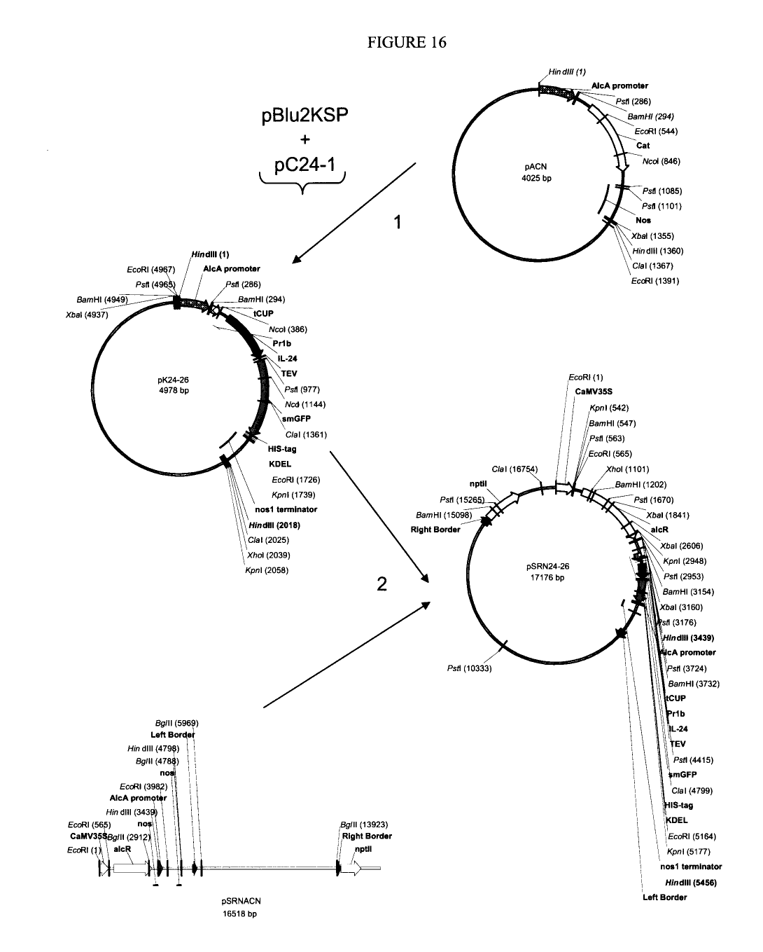

IL24-26 (SEQ ID NO:146) is a clone designed to express in a ethanol inducible manner which is constructed as per FIG. 16. As seen in FIG. 16, the IL24-26 clone consists of inducible promoter (AlcA) and terminator (nos) driving the expression of a fusion protein (SEQ ID NO:146) between IL-24 and smGFP with an ER retrieval motif (KDEL). Plasmid pACN contains the AlcA promoter with a Cat gene (Catalase) and nos terminator (a gift from Dr. A. Wang) (Ref) and was cut with BamHI and HindIII. Plasmid pC24-1 contains the CaMV 35S′ promoter, tCUP enhancer, Pr1b signal peptide, mature IL-24, TEV protease site, smGFP, HIS-tag and KDEL motif and was cut with BamHI and HindIII (see FIG. 16). The fragments of pACN and pC24-1 were ligated together into the HindIII site of the cloning vector pBlu2KSP (Stratagene) (1) treated with calf intestinal phosphatase to create the plasmid pK24-26 (FIG. 16). Plasmid pK24-26 was cut with HindIII to remove the A1cA promoter-IL-24-GFP-nos terminator cassette and the fragment was subsequently cloned into the HindIII site of binary vector pSRNACN (3) (FIG. 16) which contains the AlcR coding region (a gift from Dr. A. Wang)

(Salter et al. 1998) treated with calf intestinal phosphatase to create plasmid pSRN24-26. Note that this plasmid contains a NPT II gene conferring host plant kanamycin resistance driven by Nos promoter/terminator for transformation into Agrobacterium.

IL24-27 (AlcA Promoter-tCUP-Pr1b-IL-24::ELP::His::KDEL)

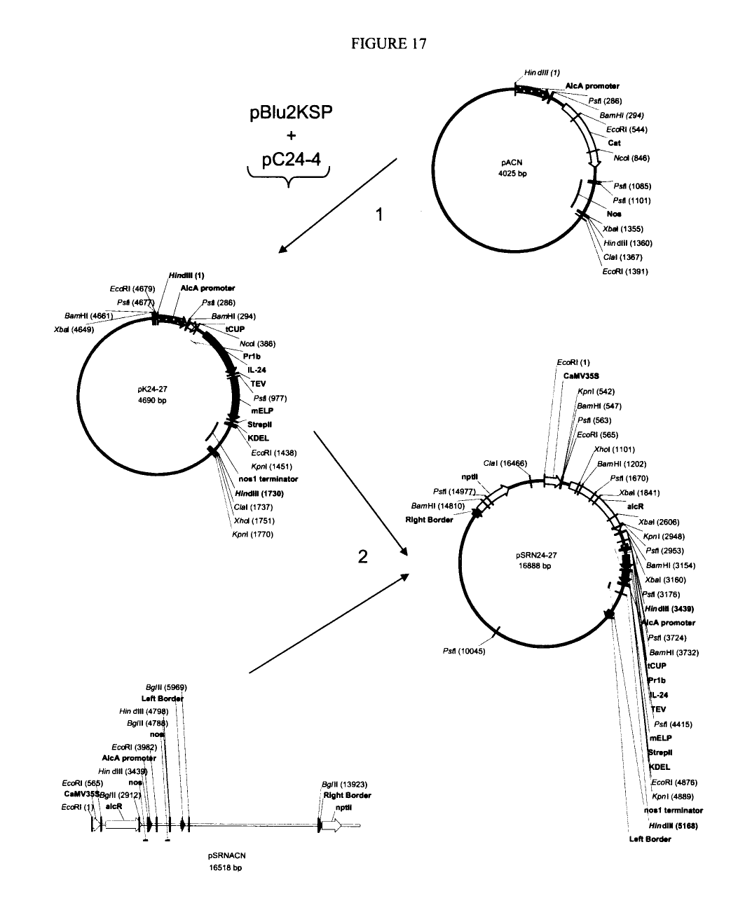

IL24-27 (SEQ ID NO:148) is a clone designed to express in a ethanol inducible manner which is constructed as per FIG. 17. As seen in FIG. 17, the IL24-27 clone consists of inducible promoter (A1cA) and terminator (nos) driving the expression of a fusion protein (SEQ ID NO:148) between IL-24 and ELP with an ER retrieval motif (KDEL). Plasmid pACN contains the AlcA promoter with a Cat gene (Catalase) and nos terminator (a gift from Dr. A. Wang) (Ref) and was cut with BamHI and HindIII. Plasmid pC24-4 contains the CaMV 35S′ promoter, tCUP enhancer, Pr1b signal peptide, mature IL-24, TEV protease site, ELP, HIS-tag and KDEL motif and was cut with BamHI and HindIII (see FIG. 17). The fragments of pACN and pC24-4 were ligated together into the HindIII site of the cloning vector pBlu2KSP (Stratagene) (1) treated with calf intestinal phosphatase to create the plasmid pK24-27 (FIG. 17). Plasmid pK24-27 was cut with HindIII to remove the AlcA promoter-IL-24-ELP-nos terminator cassette and the fragment was subsequently cloned into the HindIII site of binary vector pSRNACN (3) (FIG. 17) which contains the AlcR coding region (a gift from Dr. A. Wang) (Salter et al. 1998) treated with calf intestinal phosphatase to create plasmid pSRN24-27. Note that this plasmid contains a NPT II gene conferring host plant kanamycin resistance driven by Nos promoter/terminator for transformation into Agrobacterium.

### Example 2

### Preparation of Transgenic Low Alkaloid Nicotiana tabacum Plants

The synthetic IL-24 gene constructs were transformed into tobacco via Agrobacterium mediated transformation (Horsch et al., 1985. Science 277: 1229-1231). The Agrobacterium strain used was EHA105 carrying the disarmed Ti plasmid pEHA104. Gene constructs were transformed into N. tabacum cv. 81V9-4 low alkaloid tobacco strains according to the method described by Miki et al. (1998). Transformed and regenerated plants were initially selected for kanamycin resistance. Over forty transgenic plants were regenerated for each of constructs IL24-5 (tCUP-Pr1b::IL-24::His::KDEL), IL24-1 (tCUP-Pr1b::IL-24::TEV::smGFP::His::KDEL) and IL24-4 (tCUP-Pr1b::IL-24::TEV::ELP::StrepII::KDEL). Regenerated plants were analyzed for expression of the IL-24 protein by solid phase sandwich Enzyme-Linked ImmunoSorbent Assay (ELISA).

### Total Leaf Extract Preparation

Leaves were randomly sampled from tobacco plants that were over 30 cm in height. Six leaves were sampled, with 2 leaf discs (1.5 cm in diameter) taken in duplicate from opposite leaf sides. The six leaf discs were added to 2 ml microfuge tubes containing 3 ceramic disposable beads (BioSpec Products, Inc. Cat. No. 11079125z), flash frozen in liquid nitrogen and stored at −80 degree C. The 2 ml tubes were loaded onto a prechilled freezer blocks for the automatic tissue grinder (Retsch, Inc.). The blocks were shaken at a frequency of 30 times/sec for 2 min. The blocks were then turned inside out, and the grinding was repeated a second time. Blocks were then removed and centrifuged at 3700 rpm for 1 min to remove tissue powder from lids of the tubes. Frozen powdered tissue (approx. 60 mg of fresh weight) was vortexed for 5 sec in 400 μl of extraction buffer (phosphate buffered saline (PBS) pH 7.4, 0.05% (v/v) Tween 20, 2% (w/v) polyvinylpolypyrrolidone (PVPP), 1 mM EDTA, 1 mM phenylmethylsulfonylfluoride (PMSF), and 1 μg/ml Leupeptin). The supernatant was clarified by centrifugation at 13,000 g for 10 min at 4 degree C. The supernatant was removed to 1.5 ml tubes and re-centrifuged for an additional 10 min at 4 degree C. to remove remaining leaf debris. The supernatant was then removed to new 1.5 ml tubes and stored at 4 degree C. on ice.

### Example 3

### Quantitative ELISA for Detection of IL-24 Expression in Transgenic Tobacco Plants

Nunc Maxisorp 96-well microtiter ELISA plates were coated with anti-IL-24 mAB (R&D systems Cat. No. MAB 1965) diluted 4 μg/ml in 0.1M Na₂HPO₄, pH 9.0 using 50 μl per well; plates were incubated at 4 degree C. overnight. The plates were washed 3 times with 300 μl/well PBS-T (1 times PBS containing 0.05% Tween 20, Sigma Cat. No. P-1379). Each well was then incubated one hour at room temperature with 200 μl of blocking buffer (1% (w/v) BSA fraction V, Difco Cat. No. 232100 in PBS). The wells were washed 3 times with 200 μl/well using PBS-T. Recombinant human IL-24 standard protein (R&D systems Cat. No. 1965-IL-025) was diluted to a final concentration of 200 ng/ml in blocking buffer. Six serial 2-fold dilutions were made. The diluted standard protein and leaf extract samples (100 μl) were added to each well of the plate. Plates were then incubated at 4 degree C. overnight, washed 3 times in PBS-T and 100 μl of anti-IL-24 biotinylated polyclonal antibody (R&D systems Cat. No. BAF1965) diluted to 2 μg/ml in blocking buffer/Tween was added per well and incubated 1 hour at room temperature. The plates were washed 3 times with PBS-T. Streptavidin-HRP conjugated antibody (Vector Cat. No. A-2004) was diluted to 1:3000 in blocking buffer/Tween and 100 μl/well was added and the plates were incubated 30 min at room temperature. The plates were washed 3 times in PBS-T and 100 μl of ABTS substrate (Sigma Cat. No. A-1888) was added to each well and the plates were incubated at room temperature for approximately 5 minutes. Optical density at 415 nm wavelength was determined using a Biorad microplate reader. Data were transported and displayed using Biorad Microplate software. Linear regression and quantitation analysis were done using Microsoft Office Excel 2006. The results are presented in FIGS. 18-20.

IL24-1 Trans genic Tobacco Plants

As seen in Example 1, IL24-1 (SEQ ID NO: 67) expresses a fusion protein between mature IL-24 and smGFP (SEQ ID NO: 68) targeted to the ER via a KDEL retention motif. A 35S promoter and Nos terminator are used for constitutive expression of the construct. FIG. 18A shows the IL24-1 transgenic tobacco plant lines which survived the transition from tissue culture to soil; ranked from the highest level of IL-24 protein expression to lowest.

Seven of the highest IL-24::smGFP expressing plants were moved to the greenhouse to set seed. Leaves were sampled by ELISA analysis over several months to determine if IL-24 expression decreased over time. An untransformed 81V9 plant served as a negative control. The results are shown in FIG. 18B; letters refer to dates of sampling: A=Dec. 12, 2006; B=Feb. 6, 2007; C=Mar. 8, 2007; D=Jan. 4, 2007; E=Jan. 24, 2007. Different parts of the plant were sampled to determine if the age of the leaf also had an effect on IL-24 protein expression (WP=whole plant, leaves randomly sampled in duplicate, TL=top leaf, leaf near the top, >5 cm in length sampled in duplicate, OL=old leaf, leaf near bottom of plant, but not showing senescence, sampled in duplicate, ML=middle leaf, leaf chosen in between top leaf and old leaf). Overall, in the majority of IL24-1 transgenic tobacco plants, IL-24 protein expression decreased with time and the age of the leaf (FIG. 18B).

### IL24-4 Transgenic Tobacco Plants

As seen in Example 1, IL24-4 (SEQ ID NO:114) expresses a fusion protein between mature IL-24 and ELP (SEQ ID NO:115) targeted to the ER via a KDEL retention motif. A 35S promoter and Nos terminator are used for constitutive expression of the construct. FIG. 19A shows the IL24-4 transgenic tobacco plant lines which survived the transition from tissue culture to soil; ranked from the highest level of IL-24 protein expression to lowest.

Ten of the highest IL-24::ELP expressing plants were moved to the greenhouse to set seed. Leaves were sampled by ELISA analysis over several months to determine if IL-24 expression decreased with time. An untransformed 81V9 plant served as a negative control. The results are shown in FIG. 19B; letters refer to dates of sampling: A=Dec. 14, 2006; B=Jan. 24, 2007; C=Feb. 2, 2007; D=Mar. 8, 2007; E=Jan. 24, 2007; F=Feb. 14, 2007; and G=Dec. 12, 2007. Different parts of the plant were sampled to determine if the age of the leaf also had an effect on IL-24 protein expression (WP=whole plant, leaves randomly sampled in duplicate, TL=top leaf, leaf near the top, >5 cm in length sampled in duplicate, OL=old leaf, leaf near bottom of plant, but not showing senescence, sampled in duplicate, ML=middle leaf, leaf chosen in between top leaf and old leaf). Overall, in the majority of IL24-4 transgenic tobacco plants, IL-24 protein expression decreased with time and the age of the leaf (FIG. 19B).

### IL24-5 Transgenic Tobacco Plants

As seen in Example 1, IL24-5 (SEQ ID NO:111) expresses a mature IL-24 (SEQ ID NO:112) targeted to the ER via a KDEL retention motif. A 35S promoter and Nos terminator are used for constitutive expression of the construct. FIG. 20A shows the IL24-5 transgenic tobacco plant lines which survived the transition from tissue culture to soil; ranked from the highest level of IL-24 protein expression to lowest.

Two of the highest IL-24 expressing plants were moved to the greenhouse to set seed. Leaves were sampled by ELISA analysis over several months to determine if IL-24 expression decreased with time. An untransformed 81V9 plant served as a negative control. The results are shown in FIG. 20B; letters refer to dates of sampling: A=Feb. 14, 2007; B=Mar. 8, 2007; C=Dec. 12, 2006; D=Feb. 6, 2007. Different parts of the plant were sampled to determine if the age of the leaf also had an effect on IL-24 protein expression (WP=whole plant, leaves randomly sampled in duplicate, TL=top leaf, leaf near the top, >5 cm in length sampled in duplicate, OL=old leaf, leaf near bottom of plant, but not showing senescence, sampled in duplicate, ML=middle leaf, leaf chosen in between top leaf and old leaf). Overall, in the majority of IL24-5 transgenic tobacco plants, IL-24 protein expression decreased with time and the age of the leaf (FIG. 20B).

### Example 4

### Western Blot Analysis for IL-24 Expression in Tobacco Plants

FIG. 21.(A) Western of total leaf protein (50 μg) with biotinylated polyclonal anti-human IL-24 antibody of IL24-1 transformed plants (24-1-27) and IL24-4 transformed plants (24-4-8). TL=top leaf, leaf near the top, >5 cm in length sampled in duplicate. Untransformed 81V9 plant leaf protein (50 μg) was used as a negative control and recombinant human IL-24 protein (25 ng) was used as a positive control. (B) Western of total leaf protein (50 μg) with monoclonal anti-GFP antibody of IL24-1 transformed plants (24-1-27) and alcohol inducible IL24-26 transformed plants (24-26-21), with 10% EtOH induction (+) or without (−). TL=top leaf, leaf near the top, >5 cm in length sampled in duplicate, OL=old leaf, leaf near bottom of plant, but not showing senescence, sampled in duplicate. Untransformed 81V9 plant leaf protein (50 μg) was used as a negative control and recombinant GFP protein (25 ng) was used as a positive control.

To induce expression, leaves from IL24-26 plants (>15 cm in length, near top of plant) were removed, dipped in a solution of 10% EtOH for 10 sec with agitation, partially drip dried, and placed into MST 1 medium (pH 5.8, with MS salts and 10 g/L agar, no antibiotics) for 48 hours at room temperature in light. Control leaves (uninduced) were treated with sterile water in the same way. Leaf discs were collected and protein was extracted as described under “Total Protein Extraction.” Western blot analysis of total leaf extracts (50 μg of TSP) of plant recombinant IL-24 (pIL-24) from the top leaf of an IL24-1 transgenic plant (24-1-27 TL), the top leaf of an IL24-4 transgenic plant (24-4-8 TL), the top leaf of an IL24-26 plant (treated with 10% EtOH for induction or water for no induction) and the old leaf of an IL24-1 transgenic plant (24-1-27 OL) are shown in FIGS. 21(A) and (B) (TL=top leaf, leaf near the top, >5 cm in length sampled in duplicate, OL=old leaf, leaf near bottom of plant, but not showing senescence, sampled in duplicate). Antibodies used for detection were the BAF1965 anti-IL-24 biotinylated polyclonal antibody (R&D systems) (FIG. 21A) or a GFP polyclonal antibody (Clontech) (FIG. 21B). Recombinant human IL-24 protein (rIL-24, 25 ng) (predicted weight 19.7 kDa, but migrates at 35 kDa with glycosylation) and recombinant GFP protein (rGFP, 25 ng) (27 kDa) were used as positive controls. Untransformed 81V9 leaf extract (50 μg of TSP) was used as a negative control. The results indicate that pIL-24 is correctly produced in IL24-1 and IL24-4 transgenic plants, and induced in IL24-26 plants. The difference between the migration of the pIL-24 and hIL-24 is due to the presence of the GFP::His tag::KDEL (total fusion weight=51 kDa) or ELP::StrepII::KDEL (total fusion weight=36 kDa) sequences in the constructs IL24-1 and IL24-26 (containing GFP) and IL24-4 (containing ELP).

### Example 5

### Preparation of Transgenic Nicotiana tabacum BY-2 Cell Cultures

Three to 4 days prior to transformation, a 1 week old BY-2 culture was sub-cultured to fresh medium by adding 5 ml of the BY-2 culture into 45 ml BY-2 media. The sub-cultured flask was maintained in the dark at 28 degree C. on a shaker at 150 rpm.

BY-2 Medium Reagent Per liter was made up of: MS salts 4.3 g; MES stock (20×) 50 ml; B1 inositol stock (1000×) 1 ml; Miller's I stock 3 ml; 2,4-D (10 mg/ml) 24.3 μl; Sucrose 30 g; pH to 5.7; B1 Inositol Stock (1000×)(1 liter) Thiamine HCl(Vit B1)—1 g; MES (20×) (1 liter) MES (2-N-morpholinoethanesulfonic acid)—10 g; Myoinositol—100 mg; Miller's I (1 liter) KH₂PO₄—60 g.

Agrobacterium tumefaciens containing the expression vector of interest was streaked from a glycerol stock onto a plate of LB medium containing 50 mg/l Kanamycin and 10 mg/l Rifampicin. The bacterial culture was incubated in the dark at 27 degree C. for 48 hours. One well-formed colony was selected, and transferred to 3 ml of LB medium containing 50 mg/l Kanamycin and 10 mg/l Rifampicin. The liquid culture was incubated in the dark at 27 degree C. in an incubator shaker at 250 rpm for 24 hours

LB Medium Reagent Per liter was made up of: Bacto-tryptone 10 g; Yeast extract 5 g; NaCl 10 g; and Difco Bacto Agar 15 g.

On the day of transformation, one milliliter of 3-day old wild-type BY-2 suspension was transferred into a sterile 5-cm Petri plates. One plate was set aside to be used as a non-transformed control. Approximately, 50 μl of Agrobacterium suspension was added to the remaining plates. The plates were wrapped with parafilm then incubated in the dark at 25 C for 2 days without shaking.

Cells were transferred to a sterile, 2 ml microfuge tube, and brought up to a final volume of 2 ml with fresh BY-2 medium. They were mixed gently then centrifuged at 1000 rpm for 1 min in a cold centrifuge (4 degree C.). The supernatant was removed, and the resultant pellet was resuspended in 2 ml of liquid BY-2 media. The wash was repeated. The suspension was centrifuged, the supernatant was discarded, and the pellet was resuspended in 2 ml liquid BY-2 media. Aliquots of 1 ml were plated onto each Petri plate (10 cm) containing solid BY-2 medium (BY-2 medium solidified with 13 g/l Agar; supplemented with a final concentration of Kanamycin at 50 mg/l and Timentin at 300 mg/l, added after autoclaving). Plates were wrapped with parafilm then maintained in the dark at 25 C. After 6 to 8 weeks, putative transformants appeared. They were selected and transferred to fresh solid BY-2 medium plates (BY-2 medium solidified with 13 g/l Agar; supplemented with a final concentration of Kanamycin at 50 mg/l and Timentin at 300 mg/l, added after autoclaving). The plates were wrapped with parafilm and cultured in the dark at 25 C.

Putative transformants appeared as small clusters of callus on a background of dead, non-transformed cells. These calli were transferred to solid BY-2 medium containing Kanamycin (50 mg/l) and allowed to grow for several weeks. After 3 passes on selection medium, portions of each putative transformant were selected for ELISA analysis of IL-24 protein expression. After at least 2 runs through ELISA, lines with the highest levels of IL-24 protein were selected. The amount of callus material for each of the elite lines was then multiplied in liquid cultures.

### Example 6

### Quantitative ELISA for Detection of IL-24 Expression in Transgenic BY-2 Calli and Cell Suspension Cell Cultures

Approximately, 1 cm³ of tissue was sampled from each independent callus and flash frozen in liquid nitrogen in 1.5 ml microfuge tubes and stored at −80 degree C. Prior to sonication, tubes were placed on ice and 300 μl cold extraction buffer (see Example 2) without PVPP was added to the frozen callus. For BY-2 cells, whole wet BY-2 cells expressing IL-24 or null control were harvested directly from cell culture and excess media was removed by aspiration after centrifugation. Using 1.5 ml microfuge tubes, 500 μl packed cell volume of BY-2 cells were placed on ice with 300 μl of the cold extraction buffer. Each sample was sonicated for 15 to 20 sec on ice. Sonication was performed using a Sonic Dismembrantor Model 100™ (Fisher-Scientific, Inc.) at output control of 5 for varying amounts of time. Both callus and cell extracts were clarified by centrifugation at 13,000 g for 10 min at 4 degree C. The supernatants were then removed to new 1.5 ml tubes and stored at 4 degree C. on ice. The results are presented in FIGS. 22-26.

### IL24-1 Transgenic Calli and BY-2 Cell Suspension

As seen in Example 1, IL24-1 (SEQ ID NO: 67) expresses a fusion protein between mature IL-24 and smGFP (SEQ ID NO: 68) targeted to the ER via a KDEL retention motif. A 35S promoter and Nos terminator are used for constitutive expression of the construct. FIG. 22A shows the 26 IL24-1 transgenic BY-2 calli which survived the 3 passes on selection medium; ranked from the highest level of IL-24 protein expression to lowest.

The highest IL-24 expressing BY-2 callus was put into cell suspension culture and analyzed by ELISA for IL-24 protein expression over time. FIG. 22B shows the level of expression of IL-24 protein in the IL24-1 transgenic BY-2 calli, with letters referring to dates of sampling: A=Aug. 16, 2006; B=Nov. 29, 2006; C=Dec. 6, 2006; D=Jan. 16, 2007. FIG. 22C shows the level of expression of IL-24 protein in the IL24-1 transgenic BY-2 cell suspension cultures, where D is days after subculturing from D3=day 3 after subculturing to D7=day 7 after subculturing and letters refer to dates of subculturing: A=Nov. 22, 2006; B=Nov. 29, 2006; C=Dec. 6, 2006; D=Jan. 16, 2007. As can be seen in FIGS. 22B and C, IL-24 protein expression decreased significantly over time in both IL24-1 transgenic callus and cell suspension culture. The loss of protein expression can be seen most dramatically in the callus (FIG. 22B). The amount of IL-24 protein in BY-2 cell suspension culture varied, depending on the number of days after subculturing (FIG. 22C), however, overall a steady decline in IL-24 protein expression was also observed in suspension cell culture over time.

### IL24-3 Transgenic Calli and BY-2 Cell Suspension

As seen in Example 1, IL24-3 (SEQ ID NO:5) expresses a fusion protein between mature IL-24 and smGFP(SEQ ID NO:6) with the native 5′UTR and signal peptide and is targeted to the ER via a KDEL retention motif. A 35S promoter and Nos terminator are used for constitutive expression of the construct. FIG. 23A shows the 25 IL24-3 transgenic BY-2 calli which survived the 3 passes on selection medium; ranked from the highest level of IL-24 protein expression to lowest.

The highest IL-24 expressing BY-2 callus was put into cell suspension culture and analyzed by ELISA for IL-24 protein expression over time. FIG. 23B shows the level of expression of IL-24 protein in the IL24-3 transgenic BY-2 calli, with letters referring to dates of sampling: A=Sep. 22, 2006; B=Nov. 29, 2006; C=Dec. 6, 2006; D=Jan. 16, 2007. FIG. 23C shows the level of expression of IL-24 protein in the IL24-3 transgenic BY-2 cell suspension cultures, where D is days after subculturing from D3=day 3 after subculturing to D7=day 7 after subculturing and letters refer to dates of subculturing: A=Nov. 22, 2006; B=Dec. 6, 2006; C=Jan. 16, 2007. As can be seen in FIGS. 23B and C, IL-24 protein expression decreased significantly over time in both IL24-3 transgenic callus and cell suspension culture. The loss of protein expression can be seen most dramatically in the callus (FIG. 23B). The amount of IL-24 protein in BY-2 cell suspension culture varied, depending on the number of days after subculturing (FIG. 23C), however, an overall steady decline in IL-24 protein expression was also observed in suspension cell culture over time.

### IL24-4 Transgenic Calli and BY-2 Cell Suspension

As seen in Example 1, IL24-4 (SEQ ID NO:114) expresses a fusion protein between mature IL-24 and ELP (SEQ ID NO:115) targeted to the ER via a KDEL retention motif. A 35S promoter and Nos terminator are used for constitutive expression of the construct. FIG. 24A shows the 25 IL24-4 transgenic BY-2 calli which survived the 3 passes on selection medium; ranked from the highest level of IL-24 protein expression to lowest.

The eighth highest IL-24 expressing BY-2 callus was put into cell suspension culture and analyzed by ELISA for IL-24 protein expression over time. All the remaining high expressing IL24-4 calli did not survive to be put into culture. FIG. 24B shows the level of expression of IL-24 protein the IL24-4 transgenic BY-2 calli, with letters referring to dates of sampling: A=Aug. 22, 2006; B=Nov. 29, 2006; C=Jan. 16, 2007. FIG. 24C shows the level of expression of IL-24 protein in the IL24-4 transgenic BY-2 cell suspension cultures, where D is days after subculturing from D3=day 3 after subculturing to D7=day 7 after subculturing and letters refer to dates of subculturing: A=Nov. 29, 2006; B=Jan. 16, 2007. As can be seen in FIGS. 24B and C, IL-24 protein expression decreased significantly over time in both IL24-4 transgenic callus and cell suspension culture. The loss of protein expression can be seen most dramatically in the callus (FIG. 24B). The amount of IL-24 protein in BY-2 cell suspension culture varied, depending on the number of days after subculturing (FIG. 24C), however, an overall steady decline in IL-24 protein expression was also observed in suspension cell culture over time.

### IL24-5 Transgenic CaIli and BY-2 Cell Suspension

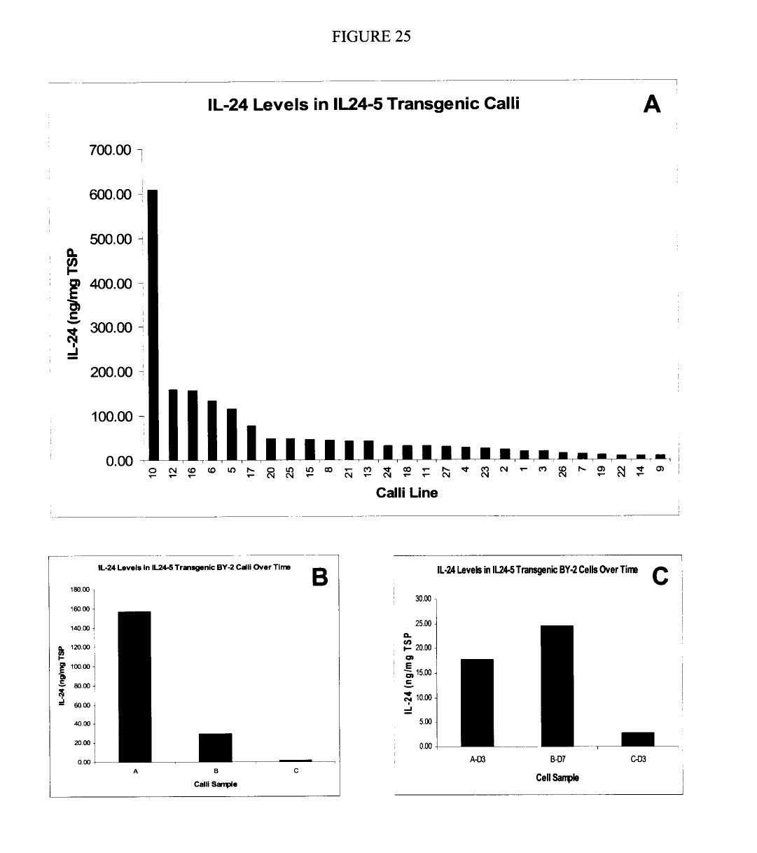

As seen in Example 1, IL24-5 (SEQ ID NO:111) expresses a mature IL-24 (SEQ ID NO:112) targeted to the ER via a KDEL retention motif. A 35S promoter and Nos terminator are used for constitutive expression of the construct. FIG. 25A shows the 27 IL24-5 transgenic BY-2 calli which survived the 3 passes on selection medium; ranked from the highest level of IL-24 protein expression to lowest.

The third highest IL-24 expressing BY-2 callus was put into cell suspension culture and analyzed by ELISA for IL-24 protein expression over time. All the remaining high expressing IL24-5 calli did not survive to be put into culture. FIG. 25B shows the level of expression of IL-24 protein in the IL24-5 transgenic BY-2 calli, with letters referring to dates of sampling: A=Aug. 23, 2006; B=Nov. 29, 2006; C=Jan. 16, 2007. FIG. 25C shows the level of expression of IL-24 protein in the IL24-5 transgenic BY-2 cell suspension culture, where D is days after subculturing from D3=day 3 after subculturing to D7=day 7 after subculturing and letters refer to dates of subculturing: A=Nov. 29, 2006; B=Jan. 16, 2007. As can be seen in FIGS. 25B and C, IL-24 protein expression decreased significantly over time in both IL24-5 callus and cell suspension culture. The loss of protein expression can be seen most dramatically in the callus (FIG. 25B). The amount of IL-24 protein in BY-2 cell suspension culture varied, depending on the number of days after subculturing (FIG. 25C), however, an overall steady decline in IL-24 protein expression was also observed in suspension cell culture over time.

### IL24-6 Transgenic CaIli and BY-2 Cell Suspension

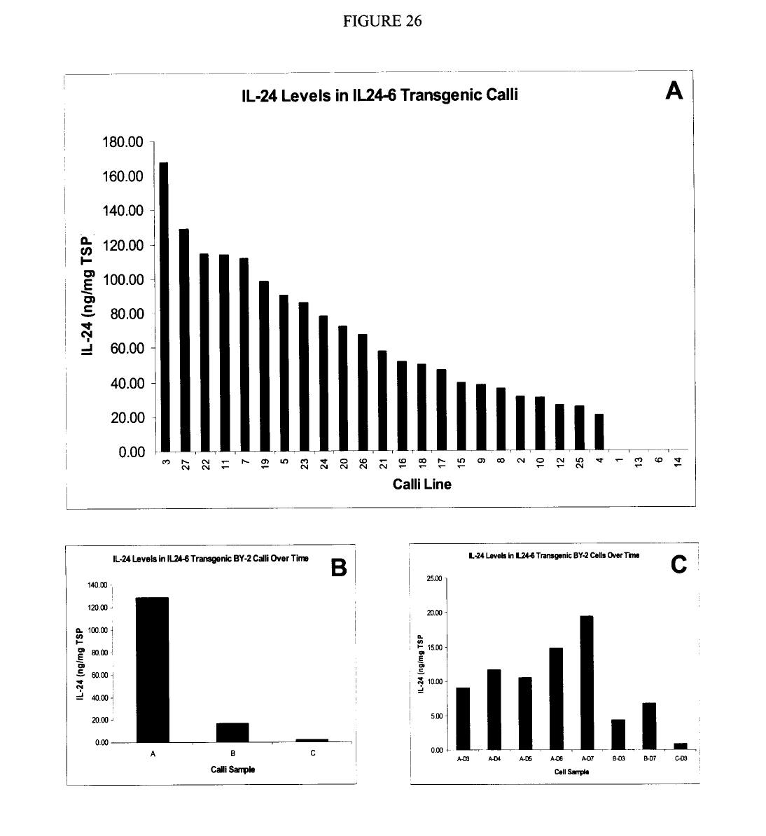

As seen in Example 1, IL24-6 (SEQ ID NO:64) expresses a fusion protein between mature IL-24 and smGFP (SEQ ID NO:65) with the native 5′UTR and signal peptide and is targeted for secretion. A 35S promoter and Nos terminator are used for constitutive expression of the construct. FIG. 26A shows the 27 IL24-6 transgenic BY-2 calli which survived the 3 passes on selection medium; ranked from the highest level of IL-24 protein expression to lowest.

The second highest IL-24 expressing BY-2 callus was put into cell suspension culture and analyzed by ELISA for IL-24 protein expression over time. The remaining high expressing IL24-6 calli did not survive to be put into culture. FIG. 26B shows the level of expression of IL-24 protein in the IL24-6 transgenic BY-2 calli, with letters refering to dates of sampling: A=Sep. 22, 2006; B=Nov. 29, 2006; C=Jan. 16, 2007. FIG. 26C shows the level of expression of IL-24 protein in the IL24-6 transgenic BY-2 cell suspension culture, where D is days after subculturing from D3=day 3 after subculturing to D7=day 7 after subculturing and letters refer to dates of subculturing: A=Nov. 22, 2006; B=Nov. 29, 2006; C=Jan. 16, 2007. As can be seen in FIGS. 26B and C, IL-24 protein expression decreased significantly over time in both IL24-6 callus and cell suspension culture. The loss of protein expression can be seen most dramatically in the callus (FIG. 26B). The amount of IL-24 protein in BY-2 cell suspension culture varied, depending on the number of days after subculturing, however, an overall steady decline in IL-24 protein expression was also observed in suspension cell culture over time.

### Example 9

### Confocal Microscopy for IL-24 Subcellular Localization

Suspension cell culture of transgenic BY-2 cells and transgenic leaf epidermal cells containing IL-24::smGFP constructs were examined for IL-24::smGFP subcellular localization by confocal laser scanning microscopy (CLSM). Cells were sampled on day 3 and day 6 after subculture. In additional, wild-type (untransformed) BY-2 cells and 81V9 leaf epidermal cells were examined as a negative control. No GFP fluorescence was observed in the negative controls. Using a packed cell volume of 100 μl in 1.5 ml microfuge tubes, cells were allowed to pack by gravity. Excess medium was removed, and the cells were diluted in 1 ml of sterile water. 200 μl of each sample was applied to a cover slip and covered with a second cover slip. Tobacco leaf epidermal cells were prepared by simply punching out leaf discs with a cork borer (diameter of 1.5 cm) and mounting them in water on cover slips with a second cover slip over top. The leaf discs were placed so that the lower epidermis faced the objective (less interference with autofluorescent chloroplasts).

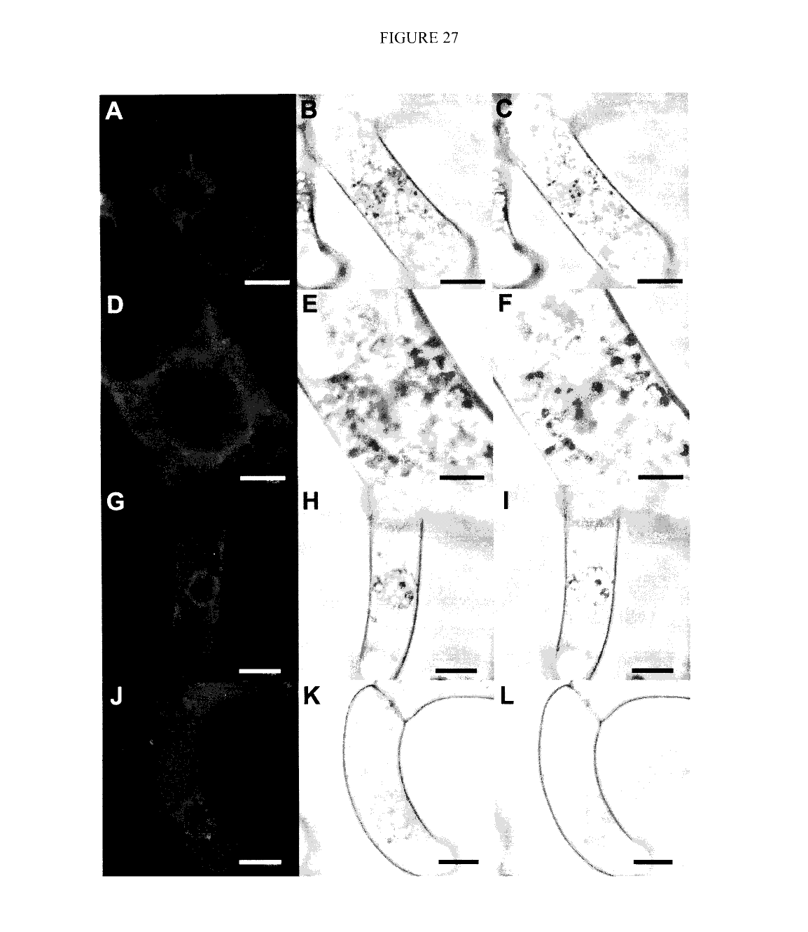

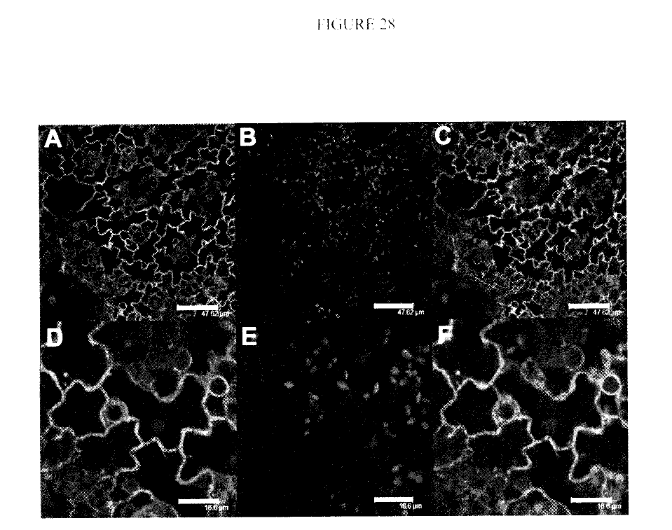

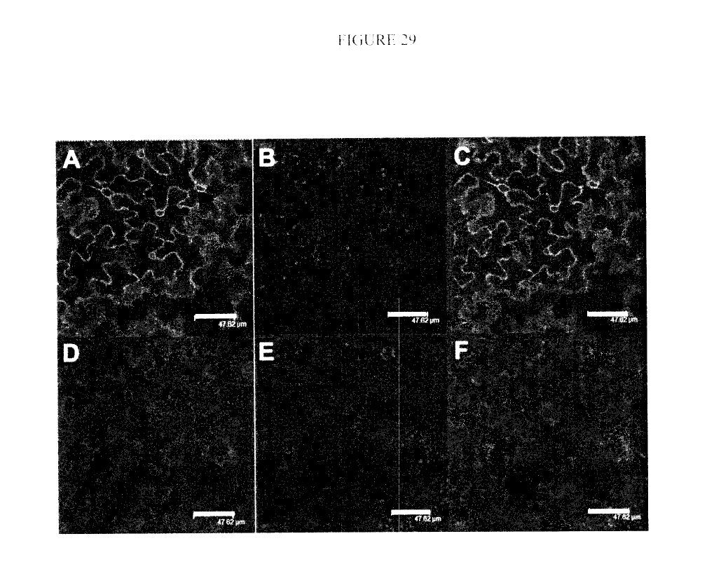

All GFP-dependent fluorescence was analyzed from transgenic BY-2 cells and tobacco leaf epidermal cells mounted in water for microscopic observations and examined with a Leica True Confocal Scanner TCS SP2 with a Leica IRE2 microscope (Leica Microsystems). For the BY-2 cells, one channel was used to collect the GFP fluorescence (controlled excitation with the 405 nm and 488 nm lights each set at 25% respectively), the other channel was used to collect transmitted light images. For leaf epidermal cells, one channel was used to collect the GFP fluorescence (controlled excitation with the 405 nm and 488 nm lights each set at 25% respectively), and the other channel was used to collect the red autofluorescence from the chloroplasts. A 63 times water immersion objective was used. The resulting micrographs can be seen in FIGS. 27 to 31.

### IL24-1 Transgenic BY-2 Cells and IL24-1 Transgenic Tobacco Leaf Epidermal Cells

The IL24:smGFP translational fusion protein was seen to be targeted correctly to the ER using the Pr1b signal peptide and KDEL ER-retrieval motif (FIG. 27) in IL24-1 BY-2 cells. On Day 3 after subculture, IL-24::smGFP protein was seen to be localized within the cortical ER network and around the nucleus (FIG. 27A-F). On Day 6 after subculture, two patterns of subcellular localization were observed; the majority of cells still show the typical ER localization (FIG. 27G-I), however, other cells show a vacuolar localization, with a hazy GFP fluorescence detected throughout the cell (FIG. 27J-L). In IL24-1 transgenic tobacco leaf epidermal cells, the IL-24::smGFP protein was detected uniformly throughout all the leaf epidermal cells (FIG. 28). The subcellular localization shows the GFP fluorescence observed within the cortical ER network and surrounding the nucleus. No vacuolar localization was observed.

### IL24-26 Transgenic Tobacco Leaf Epidermal Cells

In IL24-26 transgenic tobacco leaf epidermal cells, the IL-24::smGFP protein was detected uniformly throughout all the leaf epidermal cells (FIG. 29A-C) when IL-24 protein is induced with exposure to 10% EtOH. The subcellular localization shows the GFP fluorescence observed within the cortical ER network and surrounding the nucleus. (FIG. 29D-F) Minimal fluorescence is detected in the cells when exposed to water, as a negative control for IL-24 protein induction. No vacuolar localization was observed.

### IL24-3 Transgenic BY-2 Cells

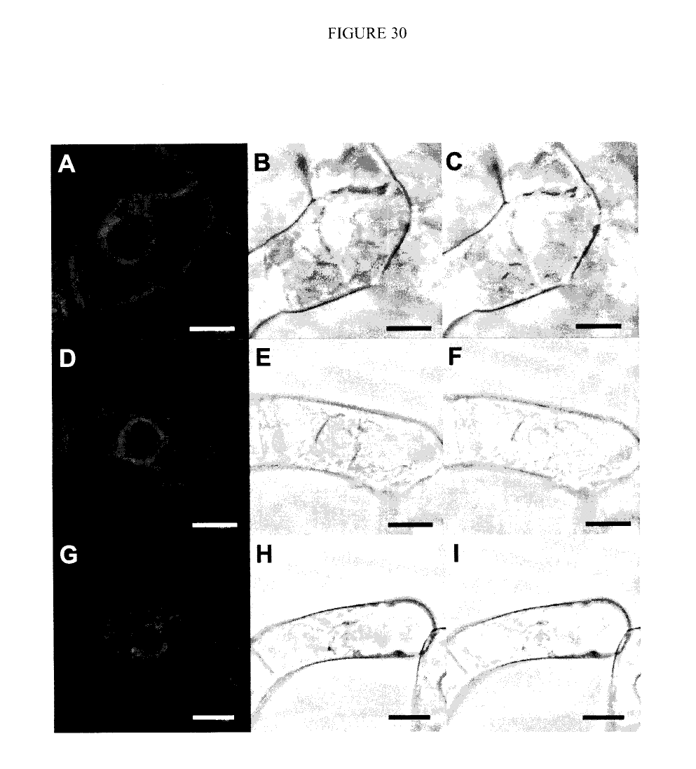

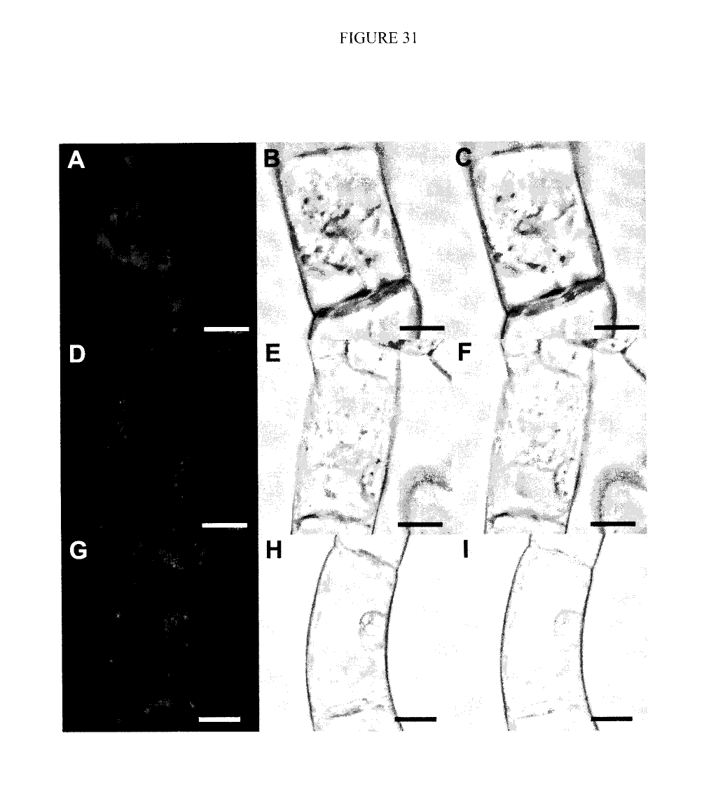

The IL24:smGFP translational fusion protein was seen to be targeted correctly to the ER using the IL-24 native signal peptide and KDEL ER-retrieval motif (FIG. 30) in IL24-3 BY-2 cells. On Day 3 after subculture, IL-24::smGFP protein was seen to be localized within the cortical ER network and around the nucleus (FIG. 30A-C). However, a small population of cells also show a vacuolar localization, with a hazy GFP fluorescence detected throughout the cell (FIG. 30D-F). On Day 6 after subculture, the majority of cells showed the vacuolar localization (FIG. 30G-I), with very few cells exhibiting the ER localization pattern.

### IL24-6 Transgenic BY-2 Cells

The IL24:smGFP translational fusion protein was targeted for secretion using the IL-24 native signal peptide (FIG. 31) in IL24-6 BY-2 cells. On Day 3 after subculture, IL-24::smGFP protein was seen to be localized in small vesicles surrounding the nucleus and the periphery of the cell (FIG. 31A-C). On Day 6 after subculture, the majority of cells still showed the vesicular localization (FIG. 31D-F), but a small population of cells exhibited a vacuolar localization, with a hazy GFP fluorescence and a small quantity of vesicles scattered throughout the cell (FIG. 31G-I).

### Example 10

### Purification of IL-24 Protein from Transgenic Leaf Tissue

### Preparation of Chelating Column

A 1-mL HiTrap Chelating HP column (GE Lifesciences) was washed with 20 mL filtered water at a rate of 2 mL/min using a laboratory pump. The column was charged with 20 mL NiSO₄ at a rate of 2 mL/min, then washed again with 20 mL of water. The column was equilibrated with 20 mL of Start Buffer (pH 8.0; 2 mM Imidazole, 20 mM Na₂HPO4, 250 mM NaCl and 50 mM Na ascorbate). Weakly bound Ni was eluted off the column with 20 mL of Elution Buffer (pH 8.0; 500 mM Imidazole, 20 mM Na₂HPO4, 250 mM NaCl and 50 mM Na ascorbate). The column was equilibrated with 20 mL of Start Buffer and was readied for sample application.

Large Scale Extraction and Purification of IL-24:GFP Protein from Leaf Material

Mature tobacco leaves (>20 cm in length), collected from 24-1-27 plants, comprising IL24-1 construct under the control of CaMV35S (tCUP-Pr1b::IL-24::smGFP::HIS::KDEL; FIG. 5), were weighed in 50 g allotments, and stored at −80° C. All necessary equipment and solutions were kept chilled at 4° C. until needed. Frozen plant tissue (50 g) was transferred to a cold blender and 200 mL of Extraction Buffer (pH 8.0; 2% PVPP, 2 mM Imidazole, 20 mM Na₂HPO4, 250 mM NaCl, 50 mM Na ascorbate, 1% of 0.1M PMSF and 0.1% of 1 mg/mL leupeptin) was added. Contents were blended for 10-15 sec, stopped to remix and blended again until a smooth slurry resulted. Plant extract was filtered through two layers of cheesecloth, then two layers of Miracloth into a cold beaker on ice. Extract was transferred to centrifuge tubes and spun at 10,000 rpm for 20 min at 4° C. Supernatant was filtered in 50 mL increments through a 0.45 μm filter to prevent protein precipitation on the column. Filtered extract was applied to the prepared column at 1.5 mL/min. A 500 μL aliquot of crude extract was saved for analysis. A 500 μL aliquot of flow through was saved for analysis. The column was washed with 20 mL of Start Buffer at 1.5 mL/min and a 500 μL start flow through aliquot was collected. The column was then washed with 20 mL of Wash Buffer (pH 8.0; 20 mM Imidazole, 20 mM Na₂HPO4, 250 mM NaCl, and 50 mM Na ascorbate) at 1.5 mL/min and a 500 μL wash flow through aliquot was collected. Samples were eluted from the column using Elution Buffer at a rate of 0.5 mL/min and eight 500 μL fractions were collected. Western blot analysis was performed on the collected flow through and eluted fractions (FIG. 32A).

Treatment with TEV Protease

The eluted fractions containing the majority of the purified IL-24:GFP protein (fractions 1-4) were pooled together and dialyzed against 1×PBS (pH 7.0) at 4° C. overnight in a Slidelyzer (Pierce, 10K MWCO) cassette. The purified IL-24:GFP protein was then incubated with recombinant AcTEV protease (Invitrogen) (1 unit/3 μg of substrate) and activation buffer (50 mM Tris-HCl (pH 8.0), 0.5 mM EDTA, 1 mM DTT) overnight at 4° C. The cleaved protein was then dialyzed again using a Slidelyzer (Pierce, 10K MWCO) cassette against 1×PBS to remove residual DTT and EDTA.

### Example 11

### Western Blot Analysis for IL-24 Purification from Tobacco Leaves

FIG. 32(A) Western of purified fractions from HiTrap Chelating column of total leaf protein extraction with biotinylated polyclonal anti-human IL-24 antibody of IL24-1 transformed plants (24-1-27). Recombinant human IL-24 protein (25 ng) was used as a positive control. (B) Western of purified IL24:GFP protein exposed to various concentrations of TEV protease (1 unit, 0.1 unit, 0.001 units) for overnight digestion at 4° C. TEV protease in buffer used as a negative control and recombinant human IL-24 protein (25 ng) was used as a positive control. (C) Western of cleaved IL-24 protein after incubation with Ni-NTA agarose beads. First panel is with polyclonal anti-human IL-24 antibody; second panel is with polyclonal anti-HIS antibody.

Western blot analysis of purified leaf extracts of plant recombinant IL-24 (pIL-24) from the mature leaves of an IL24-1 transgenic plant (24-1-27). Antibodies used for detection were the BAF1965 anti-IL-24 biotinylated polyclonal antibody (R&D systems) (FIGS. 32A and B) or a anti-HIS polyclonal antibody (GE Lifesciences) (FIG. 32C). Recombinant human IL-24 protein (rIL-24, 25 ng) (predicted weight 19.7 kDa, but migrates at 35 kDa with glycosylation) was used a as positive control. The results indicate that pIL-24 is correctly produced in IL-24-1 plants and the GFP-fusion tag can be removed leaving an intact pIL-24 protein (FIG. 32C). The pIL-24 protein does not appear to be fully glycoslyated (FIG. 32B), as the cleaved protein runs closer to the predicted molecular weight of 19 kDa, rather than the actual 35 kDa of the recombinant human IL-24 protein.

All citations are hereby incorporated by reference.

The present invention has been described with regard to one or more embodiments. However, it will be apparent to persons skilled in the art that a number of variations and modifications can be made without departing from the scope of the invention as defined in the claims.

### REFERENCES

Banerjea A, Ahmed S, Hands R E, Huang F, Han X, Shaw P M, Feakins R, Bustin S A, Dorudi S. Colorectal cancers with microsatellite instability display mRNA expression signatures characteristic of increased immunogenicity. Mol Cancer. 2004 Aug. 6; 3(1):21.

Chada S, Sutton R B, Ekmekcioglu S, Ellerhorst J, Mumm J B, Leitner W W, Yang H Y, Sahin A A, Hunt K K, Fuson K L, Poindexter N, Roth J A, Ramesh R, Grimm E A, Mhashilkar A M. MDA-7/IL-24 is a unique cytokine—tumour suppressor in the IL-10 family. Int Immunopharmacol. 2004 May; 4(5):649-67.

Cornelissen B J, van Huijsduijnen R A, Van Loon L C, Bol J F. Molecular characterization of messenger RNAs for; pathogenesis-related proteins 1a, 1b and 1c, induced by TMV infection of tobacco. EMBO J 1986, 5: 37-40

Corpet D E, Pierre F. Point: From animal models to prevention of colon cancer. Systematic review of chemoprevention in min mice and choice of the model system. Cancer Epidemiol Biomarkers Prey. 2003 May; 12(5):391-400.

Cunningham C C, Chada S, Merritt J A, Tong A, Senzer N, Zhang Y, Mhashilkar A, Parker K, Vukelja S, Richards D, Hood J, Coffee K, Nemunaitis J. Clinical and local biological effects of an intratumoral injection of mda-7(IL24; INGN 241) in patients with advanced carcinoma: a phase I study. Mol Ther. 2005 January; 11(1):149-59.

Denecke J, Botterman J, Deblaere R. Protein secretion in plant cells can occur via a default pathway. Plant Cell 1990, 2: 51-9

Fisher P B, Gopalkrishnan R V, Chada S, Ramesh R, Grimm E A, Rosenfeld M R, Curiel D T, Dent P. mda-7/IL-24, a novel cancer selective apoptosis inducing cytokine gene: from the laboratory into the clinic. Cancer Biol Ther. 2003 July-August; 2(4 Suppl 1):S23-37.

Garn H, Schmidt A, Grau V, Stumpf S, Kaufmann A, Becker M, Gemsa D, Siese A. IL-24 is expressed by rat and human macrophages. Immunobiology. 2002 July; 205(3):321-34.

Gopalan B, Shanker M, Chada S, Ramesh R. MDA-7/IL-24 suppresses human ovarian carcinoma growth in vitro and in vivo. Mol Cancer 2007, 6: 11

Guda K, Upender M B, Belinsky G, Flynn C, Nakanishi M, Marino J N, Ried T, Rosenberg D W. Carcinogen-induced colon tumors in mice are chromosomally stable and are characterized by low-level microsatellite instability. Oncogene. 2004 May 6; 23(21):3813-21.

Horsch, R. B., J. E. Fry, N. L. Hoffmann. D. Eichholtz, S. G. Rogers, and R. T. Fraley. A. Simple and general method for transferring genes into plants. Science 1985; 277: 1229-1231.

Huang E Y, Madireddi M T, Gopalkrishnan R V, Leszczyniecka M, Su Z, Lebedeva I V, Kang D, Jiang H, Lin J J, Alexandre D, Chen Y, Vozhilla N, Mei M X, Christiansen K A, Sivo F, Goldstein N I, Mhashilkar A B, Chada S, Huberman E, Pestka S, Fisher P B. Genomic structure, chromosomal localization and expression profile of a novel melanoma differentiation associated (mda-7) gene with cancer specific growth suppressing and apoptosis inducing properties. Oncogene. 2001 Oct. 25; 20(48):7051-63.

Kusnadi A R, Nikolov Z L and Howard J A. Production of recombinant proteins in transgenic plants: practical considerations. Biotechnol. Bioeng 1997, 56: 473-484.

Lebedeva I V, Sauane M, Gopalkrishnan R V, Sarkar D, Su Z Z, Gupta P, Nemunaitis J, Cunningham C, Yacoub A, Dent P, Fisher P B. mda-7/IL-24: exploiting cancer's achilles' heel. Mol Ther. 2005; 11(1):4-18.

Lebedeva I V, Su Z Z, Chang Y, Kitada S, Reed J C, Fisher P B. The cancer growth suppressing gene mda-7 induces apoptosis selectively in human melanoma cells. Oncogene. 2002 Jan. 24; 21(5):708-18.

Ma J K, Hikmat B Y, Wycoff K, Vine N D, Chargelegue D, Yu L, Hein M B, Lehner T. Characterization of a recombinant plant monoclonal secretory antibody and preventive immunotherapy in humans. Nat Med. 1998 May; 4(5):601-6.

Ma J K, Hiatt A, Hein M, Vine N D, Wang F, Stabila P, van Dolleweerd C, Mostov K, Lehner T. Generation and assembly of secretory antibodies in plants. Science. 1995 May 5; 268(5211):716-9.

Ma S W, Huang Y, Yin Z, Menassa R, Brandle J E, Jevnikar A M. Induction of oral tolerance to prevent diabetes with transgenic plants requires glutamic acid decarboxylase (GAD) and IL-4. Proc Natl Acad Sci USA. 2004 Apr. 13; 101(15):5680-5.

Ma S W, Zhao D L, Yin Z Q, Mukherjee R, Singh B, Qin H Y, Stiller C R, Jevnikar A M. Transgenic plants expressing autoantigens fed to mice to induce oral immune tolerance. Nat Med. 1997 July; 3(7):793-6.

Miki B., McHugh S G, Labbe H, Ouellet T, Tolman J H, Brandle J E. 1999. Transgenic tobacco: gene expression and applications. In Transgenic Medicinal Plants—Biotechnology in Agriculture and Forestry Vol 45. Springer, Berlin, pp 336-354

Menassa R, Nguyen V, Jevnikar A, Brandle J. A self-contained system for the field production of plant recombinant interleukin-10. Molecular Breeding 2001, 8: 177-185

Nambiar P R, Girnun G, Lillo N A, Guda K, Whiteley H E, Rosenberg D W. Preliminary analysis of azoxymethane induced colon tumors in inbred mice commonly used as transgenic/knockout progenitors. Int J Oncol. 2003 January; 22(1):145-50.

Nemunaitis J. Selective replicating viral vectors: potential for use in cancer gene therapy. BioDrugs 2003, 17: 251-62

Patel J, Zhu H, Menassa R, Gyenis L, Richman A, Brandle J. Elastin-like polypeptide fusions enhance the accumulation of recombinant proteins in tobacco leaves. Transgenic Res 2007, 16: 239-49

Ramesh R, Ito I, Gopalan B, Saito Y, Mhashilkar A M, Chada S. Ectopic production of MDA-7/IL-24 inhibits invasion and migration of human lung cancer cells. Mol Ther. 2004 April; 9(4):510-8.

Rouillard J M, Lee W, Truan G, Gao X, Zhou X, Gulari E. Gene2Oligo: oligonucleotide design for in vitro gene synthesis. Nucleic Acids Res 2004, 32: W176-80

Saeki T, Mhashilkar A, Swanson X, Zou-Yang X H, Sieger K, Kawabe S, Branch C D, Zumstein L, Meyn R E, Roth J A, Chada S, Ramesh R. Inhibition of human lung cancer growth following adenovirus-mediated mda-7 gene expression in vivo. Oncogene. 2002 Jul. 4; 21(29):4558-66.

Saito Y, Miyahara R, Gopalan B, Litvak A, Inoue S, Shanker M, Branch C D, Mhashilkar A M, Roth J A, Chada S, Ramesh R. Selective induction of cell cycle arrest and apoptosis in human prostate cancer cells through adenoviral transfer of the melanoma differentiation-associated-7 (mda-7)/interleukin-24 (IL-24) gene. Cancer Gene Ther. 2005 March; 12(3):340.

Sauane M, Gopalkrishnan R V, Sarkar D, Su Z Z, Lebedeva I V, Dent P, Pestka S, Fisher P B. MDA-7/IL-24: novel cancer growth suppressing and apoptosis inducing cytokine. Cytokine Growth Factor Rev. 2003 February; 14(1):35-51.

Sauane M, Gupta P, Lebedeva IV, Su Z Z, Sarkar D, Randolph A, Valerie K, Gopalkrishnan R V, Fisher P B. N-glycosylation of MDA-7/IL-24 is dispensable for tumor cell-specific apoptosis and “bystander” antitumor activity. Cancer Res 2006, 66: 11869-77

Serrano D, Lazzeroni M, Decensi A. Chemoprevention of colorectal cancer: an update. Tech Coloproctol. 2004 December; 8 Suppl 2:s248-52.

Sijmons P C, Dekker B M, Schrammeijer B, Verwoerd T C, van den Elzen P J, Hoekema A. Production of correctly processed human serum albumin in transgenic plants. Biotechnology (N Y) 1990, 8: 217-21

Su Z Z, Madireddi M T, Lin J J, Young C S, Kitada S, Reed J C, Goldstein N I, Fisher P B. The cancer growth suppressor gene mda-7 selectively induces apoptosis in human breast cancer cells and inhibits tumor growth in nude mice. Proc Natl Acad Sci U S A. 1998 Nov. 24; 95(24):14400-5.

Tong A W, Nemunaitis J, Su D, Zhang Y, Cunningham C, Senzer N, Netto G, Rich D, Mhashilkar A, Parker K, Coffee K, Ramesh R, Ekmekcioglu S, Grimm E A, van Wart Hood J, Merritt J, Chada S. Intratumoral injection of INGN 241, a nonreplicating adenovector expressing the melanoma-differentiation associated gene-7 (mda-7/IL24): biologic outcome in advanced cancer patients. Mol Ther. 2005 January; 11(1):160-72.

Yacoub A, Mitchell C, Brannon J, Rosenberg E, Qiao L, McKinstry R, Linehan W M, Su Z S, Sarkar D, Lebedeva I V, Valerie K, Gopalkrishnan R V, Grant S, Fisher P B, Dent P. MDA-7 (interleukin-24) inhibits the proliferation of renal carcinoma cells and interacts with free radicals to promote cell death and loss of reproductive capacity. Mol Cancer Ther. 2003 July; 2(7):623-32.

What is claimed is:

1. A plant optimized nucleic acid encoding a IL-24 polypeptide.

2. The plant optimized nucleic acid molecule of claim 1, comprising a nucleotide sequence as set forth in SEQ ID NO: 3, a nucleotide sequence that exhibits from about 70% to about 100% sequence identity with the nucleotide sequence of SEQ ID NO:3, or a nucleotide sequence that hybridizes to the nucleotide sequence of SEQ ID NO: 3, or a complement of the nucleotide sequence of SEQ ID NO: 3, under the following conditions: hybridization in 4×SSC at 65° C. for 8-16 hours, followed by one, two or three washes in 0.1×SSC at 65° C. for an hour.

3. A genetic construct comprising a regulatory region operably linked to the plant optimized nucleic acid of claim 1.

4. The genetic construct of claim 3, wherein the regulatory region is an inducible promoter.

5. The genetic construct of claim 3, further comprising a nucleotide sequence encoding a reporter protein, an elastin-like polypeptide (ELP), a targeting motif, or a combination thereof.

6. The genetic construct of claim 5, wherein the targeting motif is a peroxisome targeting signal.

7. The genetic construct of claim 4, wherein the inducible promoter is a Heat Shock Protein (HSP) promoter.

8. The genetic construct of claim 7, further comprising a nucleotide sequence encoding a reporter protein, an elastin-like polypeptide (ELP), a targeting motif, or a combination thereof.

9. The genetic construct of claim 8, wherein the targeting motif is a peroxisome targeting signal.

10. The genetic construct of claim 8 comprising SEQ ID NO: 138.

11. The genetic construct of claim 3 comprising a nucleotide sequence selected from the group consisting of: SEQ ID NO: 5, SEQ ID NO: 64, SEQ ID NO: 67, SEQ ID NO: 111, SEQ ID NO: 114, SEQ ID NO: 118, SEQ ID NO: 120, SEQ ID NO: 122, SEQ ID NO: 131, SEQ ID NO: 136, or SEQ ID NO: 138;
a) a nucleotide sequence that exhibits from about 70% to about 100% sequence identity with the nucleotide sequence of SEQ ID NO: 5, SEQ ID NO: 64, SEQ ID NO: 67, SEQ ID NO: 111, SEQ ID NO: 114, SEQ ID NO: 118, SEQ ID NO: 120, SEQ ID NO: 122, SEQ ID NO: 131, SEQ ID NO: 136, or SEQ ID NO: 138; and
b) a nucleotide sequence that hybridizes to the nucleotide sequence of SEQ ID NO: 5, SEQ ID NO: 64, SEQ ID NO: 67, SEQ ID NO: 111, SEQ ID NO: 114, SEQ ID NO: 118, SEQ ID NO: 120, SEQ ID NO: 122, SEQ ID NO: 131, SEQ ID NO: 136 or SEQ ID NO: 138, or a complement of the nucleotide sequence of SEQ ID NO: 5, SEQ ID NO: 64, SEQ ID NO: 67, SEQ ID NO: 111, SEQ ID NO: 114, SEQ ID NO: 118, SEQ ID NO: 120, SEQ ID NO: 122, SEQ ID NO: 131, SEQ ID NO: 136 or SEQ ID NO: 138, under the following conditions:
hybridization in 4×SSC at 65° C. for 8-16 hours, followed by one, two or three washes in 0.1×SSC at 65° C. for an hour.

12. A plant, a portion thereof, or a plant cell comprising the plant optimized nucleic acid of claim 1.

13. A method of producing a plant-expressed IL-24, comprising, providing a plant, a portion thereof, or a plant cell comprising the nucleic acid of claim 1, growing the plant, the portion thereof, or the plant cell, and expressing the IL-24 in the plant, a portion thereof or the plant cell thereby producing plant-expressed IL-24.

14. The method of claim 13, wherein the plant is a low nicotine, low alkaloid tobacco plant.

15. The method of claim 13, wherein after the step of expressing, the plant-expressed IL-24 is minimally processed , partially purified, or purified.

16. The method of claim 13 wherein after the step of expressing, the plant, the portion of the plant, or the plant cell is orally administered to a subject in need thereof.

17. A method of treating cancer in a subject comprising, orally administering to the subject a composition comprising plant-expressed IL-24 obtained using the method of 15.

18. The method of claim 17, wherein the cancer is colorectal cancer.
